(19) United States
(12) Patent Application Publication
(10) Pub. No.: US 2025/0011734 A1
(43) Pub. Date: Jan. 9, 2025

(54) DGAT1/2-INDEPENDENT ENZYME SYNTHESIZING STORAGE LIPIDS (DIESL)

(71) Applicant: STICHTING HET NEDERLANDS KANKER INSTITUUT-ANTONI VAN LEEUWENHOEK ZIEKENHUIS, Amsterdam (NL)

(72) Inventors: Gian-Luca MCLELLAND, Amsterdam (NL); Thijn Reinout BRUMMELKAMP, Amsterdam (NL)

(21) Appl. No.: 18/708,092

(22) PCT Filed: Nov. 9, 2022
(86) PCT No.: PCT/NL2022/050633

§ 371 Date: May 7, 2024

(30) Foreign Application Priority Data
Nov. 9, 2021 (NL) 2029680

(51) Int. Cl. C12N 9/10; A01K 67/0276; C12N 9/90; C12P 7/6445; C12Q 1/48; C12Q 1/533

(52) U.S. Cl. CPC C12N 9/1029; A01K 67/0276; C12N 9/90; C12P 7/6445; C12Q 1/48; C12Q 1/533; C12Y 503/04001; A01K 2217/075; A01K 2227/105

(57) ABSTRACT

The present invention is in the field of triacylglyccrol synthesis. In particular the invention relates to the identification of DIESL as a new triacylglycerol synthase. The invention also relates to the identification of TMX1 as a negative regulator of DIESL mediated triacylglycerol synthesis. Screening assays for identifying agents that modulate the activity of DIESL and/or of TMX1, or that modulate the interaction between DIESL and TMX1 are also provided.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Triacylglycerols (TAGs) are quantitatively the most important storage form of energy for eukaryotic cells, fueling mitochondrial beta-oxidation. In humans, TAGs are synthesized from excess fatty acyl-CoAs by diacylglycerol O-acyltransferases (DGAT1/DGAT2). As such, TAG synthesizing enzymes play a fundamental role in the metabolism of diacylglycerol and are important in higher eukaryotes for intestinal fat absorption, lipoprotein assembly, fat storage in adipocytes, and milk production. In addition, TAGs have been implicated in various conditions including obesity, diabetes, insulin resistance and cardiovascular disease, and inhibitors of DGAT1 are under research for the treatment of metabolic disorders, e.g. obesity. Type 2 diabetes, and insulin resistance syndrome (or metabolic syndrome) and other associated or related diseases and conditions (see, for example, US2019135829 and WO2007126957).

The two enzymes held responsible for TAG synthesis, DGAT1 and DGAT2, catalyze the same reaction (condensing diacylglycerol and fatty acyl-CoA to form TAGs), but they are evolutionarily unrelated and are markedly different in their structures, substrate preferences, subcellular localizations, and physiological roles. DGAT1 is a member of the membrane-bound O-acyltransferase gene family. DGAT is exclusively localized in the ER, where it catalyzes the esterification reaction between its substrates, a fatty acyl-CoA and an acyl acceptor, to generate a neutral lipid. DGAT1 can esterify a variety of substrates, including diacylglycerol and monoacylglycerol, although TAG synthesis is the predominant function of DGAT1. DGAT1 is believed to have an important role in detoxification of excess lipids taken up from outside the cell and play a role in preventing activation of ER stress pathways as the consequence of excess (lipotoxic) lipids.

DGAT2 is a member of the DGAT2 gene family. In contrast to DGAT1, DGAT2 is localized to both the ER and Lipid Droplet organelles (LDs). In contrast to DGAT1, DGAT2 lacks multiple transmembrane domains but has a hydrophobic sequence that is embedded in the ER bilayer.

In agreement with the different properties of both enzymes, mice lacking either DGAT1 or DGAT2 have different phenotypes. DGAT1 knockout mice are viable, have body fat, but are lean. They appear to be resistant to diet-induced obesity, diabetes, and liver steatosis. In addition, these mice live on average ˜25% longer than control mice. Mice lacking DGAT2 die shortly after birth, mainly because of a lipid defect in the skin impairing its barrier function.

Because of TAGs central role in a variety of cellular and metabolic processes, including these mentioned above, there remains interest in the role of TAGs and the TAG synthesizing enzymes in, for example, health and diseases. As such, there is much interest in the identification of polynucleotides encoding proteins having TAG synthase activity, as well as the proteins encoded thereby, and the use thereof in various applications.

Accordingly, the technical problem underlying the present invention can been seen in the provision of such polynucleotides, polypeptides, uses thereof, as well a screening methods for identifying, for example, agents that modify activity of the identified polynucleotides, and as such for complying with any of the aforementioned needs. The technical problem is solved by the embodiments characterized in the claims and herein below.

### DESCRIPTION

### Drawings

Embodiments of the invention are further described hereinafter with reference to the accompanying drawings, in which:

FIG. 1: TMX1 suppresses alternative TAG accumulation. (A) Schematic representation of a haploid genetic screen in DGAT DKO cells; LO and HI represent the 5% of cells with the lowest and highest fluorescent signal, respectively. (B) Fishtail plot depicting genetic regulators of lipid droplets in DGAT DKO cells. Significant positive and negative regulators are indicated. MI, mutational index. (C) Western blot of TMX1 levels in HAP1 cell lines. (D) lipid droplets, visualized by BODIPY 665/676, in HAP1 cell lines. Hoechst 33342, (blue); scale bar, 10 microns. (E) Quantitative increase in BODIPY 493/503 by TMX1 KO in HAP1 cells as measured by flow cytometry. (F) TLC of neutral lipids in HAP1 cell lines. Cells were additionally pulsed with 50 UM oleic acid for 24h. CE, cholesteryl ester; TAG, triacylglycerol; DAG, diacylglycerol. (G) lipid droplets (as visualized by BODIPY 665/676) in 293T ATMX1 cells. Hoechst 33342, blue; scale bar, 10 microns. (H) TLC of neutral lipids in 293T ATMX1 cells, treated with 10 μM (each) DGATi and/or 50 μM oleic acid for 24h.

FIG. 2: DIESL drives TAG accumulation in the absence of TMX1. (A) Schematic representation of the DGAT pathway and the putative TMX1-inhibited pathway. (B) Setup of modifier screens to identify differential regulators of each pathway. (C) Fishtail plots of lipid droplet screens in HAP1 WT cells treated with oleic acid (left) and in HAP1 ATMX1 cells (right). Significant positive and negative regulators are again indicated. MI, mutational index. (D) Difference in mutational index (log 2) between both screens for every gene with at least 30 insertions in each screen. (E) Western blot of TMX1 in HAP1 WT, ΔDIESL and DGAT DKO cells transduced with sgTMX1. (F) Ultrastructural analysis of lipid droplets (arrowheads) in WT and ΔDIESL cells after TMX1 loss. Scale bar, 1 micron. (G) lipid droplets, visualized by BODIPY 665/676, in WT, ΔDIESL and DGAT DKO cells transduced with the indicated sgRNA or treated with 200 UM oleic acid for 24h. Scale bars, 10 microns. (H) TLC analysis of neutral lipids in HAP1 DGAT DKO cells additionally lacking DIESL and/or TMX1.

FIG. 3: The TMX1-DIESL enzymatic complex. (A) Western blot analysis of TMX1 and HA-tagged DIESL (3xHA-DIESL) in HAP1 cells lacking endogenous DIESL (and TMX1), with or without crosslinking by 1% PFA. (Red) arrowheads indicate the DIESL-TMX1 heterodimer, and * indicates a non-DIESL band. (B) Co-immunoprecipitation of TMX1 with DIESL from rescued HAP1 cells (* indicate antibody chains). (C) Structure of PIsC from T. maritima (left, PDB ID 5KYM) and PHYRE2-derived homology model of human DIESL (center). Membrane-embedded helices, (orange); conserved catalytic stretch, (salmon). Comparison of active sites (right) of PlsC and DIESL, indicating catalytic histidines and aspartates. (D) Sequence conservation of catalytic dyads across acyltransferases (accession numbers in parentheses). (E) Western blot of HAP1 cells rescued with catalytic-active and-dead (H130A) DIESL (* indicates a non-specific band). (F) Analysis of lipid droplets (BODIPY 493/503 intensity) in DIESL-rescued HAP1 cells by flow cytometry.

FIG. 4: DIESL is a non-canonical TAG synthase. (A) TLC separation of neutral lipids and immunoblot analysis of HAP1 cell lines reconstituted with 3xHA-DIESL (* indicates a non-specific band). (B) Absolute lipid abundance in HAP1 4KO cells (ΔDGAT1ΔDGAT2ΔDIESLΔTMX1) reconstituted with 3xHA-DIESL WT or H130A as determined by shotgun lipidomics. CE, cholesteryl ester; TAG, triacylglycerol. (C) Relative abundance of triacylglycerol (TAG), diacylglycerol (DAG) and phosphatidylcholines ([e] PCs, which include ether-linked PCs) in HAP1 4KO cells reconstituted with 3xHA-DIESL (top). **, p<. 01; ****, p<. 0001 (two-way ANOVA, Bonferroni correction). (D) Fold-change plot of the change in abundance of all lipid species between HAP1 4KO cells expressing WT vs. H130A DIESL as determined by lipidomics. Significant (FDR-corrected p<. 05) changes in ePC (orange and red circles; left) and TAG (blue circles; right) species are indicated. Circle size depicts the relative, scaled abundance of the indicated lipid species. (E) Acylation assay of NBD-DAG. HAP1 cell lines were treated with 25 μM NBD-DAG for 1h prior to TLC analysis of polar lipids (to accommodate the charge on the NBD group). NBD-tagged lipids were identified by NBD fluorescence. (F) Schematic representation (top-left) of human DIESL constructs for recombinant expression in E. coli. The pelB signal sequence is indicated; a black arrowhead indicates the cleavage site. TLC separation of neutral lipids and immunoblot analysis (right) from intact E. coli expressing the indicated construct, induced for 16 h at 25° C. Absolute abundance of TAG species (bottom-left) from E. coli expressing the indicated construct. (G) Lipid fingerprints of acyl chains present in TAGs (left) and phospholipids (right) from whole-lipidome analyses of E. coli (top) and HAP1 4KO cells (bottom). The TAG plots represent (ss-) DIESL WT-expressing cells and the phospholipid plots represent the control condition; either E. coli expressing empty vector or HAP1 4KO expressing DIESL H130A. Encircled (orange) circles represent lipids common to both E. coli and human cells, whereas blue (other) circles were only detected in HAP1 cells. Circle size depicts the relative, scaled abundance of the indicated lipid species. (H) TAG synthesis is accomplished by two pathways in higher eukaryotes; the DGAT pathway (green, left in the graphic) acylates DAG using exogenously-derived fatty acyl-CoA (FA-CoA) while the TMX1-DIESL pathway (orange, right in the graphic) utilizes endogenous fatty acyl chains, potentially derived from the sn-2 position of ePCs (e.g., derived from membrane phospholipids or their precursors).

FIG. 5: A residual TAG pool persists in the absence of DGAT enzymes. (A) Schematic representation of DGAT1-and DGAT2-specific inhibitors. (B) TLC separation of neutral lipids from HAP1 cells treated with 50 μM oleic acid or 10 μM (each) DGATi for 16h. CE, cholesteryl ester; TAG, triacylglycerol; DAG, diacylglycerol; FFA, free fatty acid; OA, oleic acid. (C) TLC analysis of neutral lipids from HAP1 and 293T cells treated with 10 UM (each) DGATi for 72h. (D) Sequencing peaks of the mutated DGAT1 and DGAT2 loci from a HAP1 DGAT DKO clone where a blasticidin resistance (BLASTres) cassette was integrated at each locus. Arrows represent the direction of amplification. (E) TLC analysis of neutral lipids from HAP1 WT and DGAT DKO cells. (F) Panel of cell lines treated with 200 UM oleic acid for 24h. Neutral lipid loading is visualized by lipid droplets stained with BODIPY 493/503. Scale bars for HAP1 cells, 10 microns; scale bars for other cell lines, 20 microns.

FIG. 6: TMX1 loss leads to TAG accumulation. (A) Quantification of lipid droplets (LDs) in HAP1 WT (black) cells, as well as two TMX1 KO clones (green). lipid droplets per cell (left), lipid droplet area (center) and the distribution of lipid droplet sizes (right) are shown. ****, p<. 0001 compared to WT (two-way ANOVA, Bonferroni correction). (B) Ultrastructural analysis of lipid droplets (red arrowheads) in HAP1 WT and TMX1 KO cells. Scale bars, 1 micron. (C) TLC analysis of neutral lipids and Western blot analysis of proteins extracted from A549 (left) and U2OS (right) cells, transduced with Cas9 and a control sgRNA (sgCTRL) or sgRNA targeting TMX1 (sgTMX1), treated with 10 M (each) DGATi for 72 hours/pct

FIG. 7: TAG suppression is unique to TMX1. (A) Confocal imaging of TMX1 (green) and PDI (ER marker, red) in HAP1 cells. Hoechst 33342, blue; scale bar, 10 microns. (B) Relative expression of TMX family members in HAP1 cells as determined by RNAseq. (C) Schematic representation of topology of TMX family members (top), as well as domain structure (bottom). Location of antibody binding and sequence homology to TMX1 are indicated. (Yellow and red) Circles represent catalytic cysteine and serine residues, respectively. ss, signal sequence; V5, simian virus 5 epitope tag; TXN, thioredoxin domain; TM, transmembrane helix. (D) Western blot analysis of HAP1 DGAT TMX1 3KO cells expressing V5-tagged TMX family members depicted in C. (E) TLC analysis of neutral lipids in cells from D. (F) Confocal imaging of lipid droplets (stained with BODIPY 665/676) in cells from D. Scale bar, 10 microns.

FIG. 8: Genetics of lipid droplet regulation. Genes related to the titular cellular processes are indicated in the lipid droplet screens performed in oleic acid-loaded HAP1 WT cells (left column) and HAP1 ATMX1 cells (right column). Significant (FDR-corrected p<. 05) positive and negative regulators are again indicated (colored light blue and orange).

FIG. 9: DIESL is an ER membrane glycoprotein. (A) Localization of 3xHA-DIESL (green) in Hela cells co-stained with the ER marker CANX (red). Hoechst 33342, blue; scale bar, 10 microns. (B) Crude microsomes from HAP1 cells rescued with 3xHA-DIESL were treated with increasing amounts of proteinase K in a protease protection assay, and proteins were detected by Western blot analysis. The black arrowhead indicates the full-length DIESL, whereas the red arrowhead indicates C-terminal degradation products. (C) An N-glycosylation motif (N—X-S/T) is conserved at the DIESL N-terminus. (D) Deglycosylation of DIESL N-glycans by PNGaseF. Glycosylation was assessed by Western blot, where LAMP1 served as a positive control. (E) HAP1 cells rescued with 3xHA-DIESL where treated with 5 μg/ml brefeldin A for the indicated period of time, and DIESL glycosylation status was assessed by Western blot. The N5Q mutant served as a negative control.

FIG. 10: TMX1 and DIESL physically interact. (A) HAP1 cells rescued with 3xHA-DIESL were subcloned, and then transduced with Cas9 and a control sgRNA (sgCTRL) or an sgRNA targeting TMX1 (sgTMX1). DIESL levels were assessed by Western blot. (B) Hela cells expressing 3xHA-DIESL were transduced as in A. Cells were crosslinked with PFA prior to lysis and Western blot analysis. x and m indicate the cross-linked TMX1-DIESL dimer and corresponding monomer, respectively. (C) TMX1 was co-immunoprecipitated from HAP1 cells rescued with DIESL in a buffer containing the indicated detergent (* indicates an antibody chain).

FIG. 11: DIESL homology modeling and residue conservation. (A) Homology modeling of human DIESL using PHYRE2 (blue) and iTASSER (orange). Dark grey portions indicate unmodeled regions of the protein. (B) Sequence conservation of the active site histidine (H130, blue boxes) and aspartate (D136, red boxes), as well as the phosphate-coordinating arginine (R201, blue boxes) and cysteine residues present in human DIESL (C8, C20, C152, C174 and C183; yellow boxes) across species. Black boxes indicate a lack of conservation, and sequence identity to human DIESL is indicated. Hs, Homo sapiens; Rn, Rattus norvegicus; Bt, Bos taurus; Dm, Drosophila melanogaster; Ce, Caenorhabditis elegans.

FIG. 12: The residual TAG pool is synthesized by DIESL. TLC analysis of neutral lipids extracted from HAP1 DGAT DKO and DGAT DIESL 3KO cells.

FIG. 13: Perturbation of the lipidome by DIESL. (A) Schematic representation of the lipidomic analysis performed in HAP1 cells lacking DGAT1, DGAT2, DIESL and TMX1 (4KO) in which active (WT) or catalytic-dead (H130A) DIESL was reintroduced. (B) Lipidome of 4KO cells, either naïve (control) or expressing WT or H130A DIESL. Each species is represented as percent of the total lipid abundance in the respective samples, where 1292 unique lipid species were detected. All significant differences between WT and H130A cells are indicated (two-way ANOVA, Bonferroni correction). **, p<. 01; ****, p<. 0001. CE, cholesteryl ester; TAG, triacylglycerol; DAG, diacylglycerol; PA, phosphatidic acid; (e) PC, (ether-linked) phosphatidylcholine; (e) PE, (ether-linked) phosphatidylethanolamine; PG, phosphatidylglycerol; PI, phosphatidylinositol; PS, phosphatidylserine; CL, cardiolipin; SM, sphingomyelin; (Hex) Cer, (hexosyl) ceramide; LPL, lysophospholipid. (C) Relative abundance of ePC and PC species (grouped by acyl chain length and saturation) in HAP1 4KO cells expressing WT and H130A DIESL, expressed as a fraction of the levels in H130A cells. (D) Distribution of ePC, PC and phospholipid (PL) species (grouped by acyl chain length and saturation) in HAP1 4KO cells expressing WT and H130A DIESL, expressed as a percentage of the total amount of that species detected in that condition. (F) Distribution of ePC, PC and phospholipid (PL) species, grouped by sn-2 chain length (left) and saturation (right), in HAP1 4KO cells expressing WT and H130A DIESL. ns, not significant; **, p<. 01; ****, p<. 0001 (two-way ANOVA, Bonferroni correction).

FIG. 14: DAG acylation by DIESL. (A) Complete TLC plate from FIG. 4E. (B) Structure of NBD-DAG. (C) Analysis of NBD-DAG acylation (by NBD fluorescence) by TLC separation of polar lipids. HAP1 WT, DGAT DKO and DGAT DIESL 3KO cells were incubated with 25 UM NBD-DAG in the presence of absence of 50 UM oleic acid for one hour prior to lipid extraction. (D) Confocal imaging of HAP1 DGAT TMX1 3KO labeled with 50 μM TopFluor-DAG. After fixation, membranes were extracted with 0.1% TX-100 and lipid droplets were subsequently stained with BODIPY 665/676. Hoechst 33342, blue; scale bar, 5 and 1 microns.

FIG. 15: Steady-state DIESL activity in 293T and U251 cells. (A) TLC analysis of neutral lipids extracted from 293T WT cells, as well as two DIESL KO clones, treated with 10 UM (each) DGATi for 48h. (B) TLC analysis of neutral lipids extracted from U251 cells, transduced with the indicated sgRNA, treated with 10 μM (each) DGATi for 96h.

FIG. 16: Table summarizing sgRNAs (expressed as the 20-nucleotide target sequence upstream of the PAM sequence) used to target the indicated genetic locus.

FIG. 17: Table summarizing primers used to amplify genetic loci in order to study genetic modifications in mutated cell lines.

FIG. 18: Catalogue of mutated loci from cell lines used in this study. Insertions and deletions (indels) were determined from Sanger sequencing. Although this is not indicated in the table, in instances where a blasticidin (BLAST) or puromycin (PURO) resistance cassette was inserted at the indicated locus, integrations were also confirmed via sequencing as well. Genetic modifications are presented in a red font, with deletions represented as “:”. The TMX1 and DIESL loci in 293T cells are inferred to be triploid and diploid, respectively, as several other clones (not used in this study) were ascertained to be compound heterozygotes by TIDE analysis.

FIG. 19: Physiological roles of DIESL. (A) Body weight of adult (22-28 weeks) mice of the indicated genotype and sex. Bars represent mean±SEM of n=8 to 15 mice (one-way ANOVA, Bonferroni correction; ns, not significant). (B) TAG content in serum (left, determined by assay) and liver (right, determined by mass spectrometry) of, respectively, adult and 6-week-old male mice of the indicated genotype. Bars represent mean±SEM of n=20 to 24 and 4 mice, respectively (two-tailed Student's t-test; ns, not significant). (C) Immunoblot analysis of CHOP in liver lysates collected from 6-week-old mice (4 mice per genotype). (D) Levels of phosphatidic acid (PA), diacylglycerol (DAG), phosphatidylcholine (PC) and phosphatidylethanolamine (PE) in the livers of 6-week-old mice as determined by mass spectrometry. Bars represent mean±SEM of n=4 mice (two-way ANOVA, Bonferroni correction; ns, not significant). (E) Immunoblot analysis of subcellular fractions obtained from HAP1 ΔDIESL cells reconstituted with 3xHA-DIESL. hom., homogenate; P100k, 100,000g pellet; S100k, 100,000g supernatant; mito., mitochondria; MAM, mitochondria-associated membrane of the ER. (F) Determination of mitochondrial membrane potential (Aw, left) and ATP levels (right) in RPE1 cells expressing Cas9 and the indicated sgRNA, cultured in either complete medium (“control”), medium lacking lipoproteins or lipoprotein-deficient medium supplemented with 50 UM oleic acid for 24 hours. Bars represent mean±SEM of n=3 and 4 independent experiments, respectively (two-way ANOVA, Bonferroni correction; ns, not significant). (G) Brightfield images (left) and quantification of viability (right) of RPE1 from f. Bars represent mean±SEM of n=3 independent experiments (two-way ANOVA, Bonferroni correction). (H) Brightfield images of 293T cells (left), and quantification of cell number of 293T cells (centre) and U251 cells (right) cultured under the indicated conditions for 24 hours. Bars represent mean±SEM of n=3 independent experiments (two-way ANOVA, Bonferroni correction; ns, not significant).

### DEFINITIONS

A portion of this disclosure contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a”, “an”, and “the” include plural referents unless the context clearly dictates otherwise. Thus, for example, reference to “a cell” includes a combination of two or more cells, and the like.

As used herein the term “about” and “approximately”, when referring to a measurable value such as an amount, a temporal duration, an activity, and the like, is meant to encompass variations of ±20%, ±10% more preferably ±5%, even more preferably ±1%, still more preferably ±0.1% from said measurable value, in such way the variations are appropriate to perform the disclosed methods.

As used herein, the term “and/or” refers to a situation wherein one or more of the stated cases may occur, alone or in combination with at least one of the stated cases, up to with all of the stated cases.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, etc. As used herein, the term “at most” a particular value means that particular value or less. For example, “at most 5” is understood to be the same as “5 or less” i.e., 5, 4, 3, . . . 10,-11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, “conventional techniques” or “methods known to the skilled person” refer to a situation wherein the methods of carrying out the conventional techniques used in methods as disclosed herein will be evident to the skilled worker. The practice of conventional techniques in molecular biology, biochemistry, cell culture, genomics, sequencing, medical treatment, pharmacology, immunology and related fields are well-known to those of skill in the art and are discussed, in various handbooks and literature references.

As used herein, “exemplary” means “serving as an example, instance, or illustration,” and should not be construed as excluding other configurations disclosed herein.

As used herein “candidate agent” or “agent” refer to a molecule that may be screened for, or be identified as, modulating activity of a target activity (e.g. TAG synthase activity of a polypeptide as disclosed herein). Such agent may, for example, be an inhibitor or enhancer of the activity and may find use in a variety of applications, including as therapeutic agents, as agricultural chemicals, and so on. The screening methods will typically be assays which provide for qualitative/quantitative measurements of the activity in the presence of a particular candidate agent. For example, the assay could be an assay which measures the TAG synthase activity of a DIESL polypeptide in the presence and absence of a candidate agent, or which measures modulation of DIESL polypeptide and/or TMX1 polypeptide expression or interaction between a DIESL polypeptide and a TMX1 polypeptide in the presence of a particular agent. The screening method may be an in vitro or in vivo format, and both formats are readily developed by those of skill in the art.

(Candidate) agents may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts or purified compounds are available or may be produced. Additionally, natural or synthetically produced libraries and compounds can be prepared using conventional chemical, physical and biochemical means, and may be used to produce combinatorial libraries. Known pharmacological agents may be subjected to directed or random chemical modifications, such as acylation, alkylation, esterification, amidification, etc. to produce structural analogs or derivates. (Candidate) agents may also be biomolecules including peptides, saccharides, fatty acids, steroids, purines, pyrimidines, derivatives, structural analogs or combinations thereof.

Using the above screening methods, a variety of different therapeutic agents may be identified. Such agents may target an enzyme itself, or an expression regulator factor thereof. Such agents may be inhibitors or promoters of the targeted activity, where inhibitors are those agents that result in at least a reduction of activity as compared to a control and enhancers result in at least an increase in activity as compared to a control. Such agents may be used in a variety of (therapeutic) applications.

As used herein, the term “determining”, for example determining activity, production, and/or amounts includes measuring, analyzing, estimating, following, and the like of such activity, production, and/or amounts, for example, using conventional means and/or techniques. Likewise, the term “providing”, for example, providing a cell includes preparing, isolating, obtaining, and the like, of such cell.

As used herein, “expression” or “expression of a gene” refers to the process wherein a DNA region which is operably linked to appropriate regulatory regions, particularly a promoter, is transcribed into an RNA, and, in case the RNA encodes for a biologically active protein or peptide, subsequently translated into a biologically active protein or peptide.

As used herein, the terms “host cell,” “host cell line,” and “host cell culture” are used interchangeably and refer to cells into which exogenous nucleic acid (polynucleotides) and/or exogenous polypeptide has been introduced, including the progeny of such cells. Host cells may include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein. Within the context of the present invention it will be clear for a skilled person if reference to a “cell” includes reference to a “host cell”.

As used herein “identity” or “sequence identity” refers to the degree of relatedness between two or more amino acid sequences, or two or more nucleic acid sequences (polynucleotide sequences), as determined by comparing the sequences. The comparison of sequences and determination of sequence identity may be accomplished using a mathematical algorithm; those skilled in the art will be aware of computer programs available to align two sequences and determine the percent identity between them. The skilled person will appreciate that different algorithms may yield slightly different results.

Thus, the “percent identity” between a query nucleic acid sequence and a subject nucleic acid sequence is the “identities” value, expressed as a percentage, that is calculated by, for example, the BLASTN algorithm when a subject nucleic acid sequence has 100% query coverage with a query nucleic acid sequence after a pair-wise BLASTN alignment is performed. Such pairwise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein.

Similarly, the “percent identity” between a query amino acid sequence and a subject amino acid sequence is the “identities” value, expressed as a percentage, that is calculated by the BLASTP algorithm when a subject amino acid sequence has 100% query coverage with a query amino acid sequence after a pair-wise BLASTP alignment is performed. Such pairwise BLASTP alignments between a query amino acid sequence and a subject amino acid sequence are performed by using the default settings of the BLASTP algorithm available on the National Center for Biotechnology Institute's website with the filter for low complexity regions turned off. Importantly, a query amino acid sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

As used herein, the term “isolated” when referring to a polynucleotide (nuclei acid) or polypeptide (protein), refers to proteins or nucleic acids being present in a non-naturally occurring environment, e. g. are separated from their naturally occurring environment. For example, an isolated protein or polypeptide according to the invention relates to a protein which is no longer in its natural environment, for example, in vitro or in a recombinant host cell. The terms, next to being isolated from naturally occurring source, also refers to such protein or nucleic acid being artificially or synthetically produced. Within the context of the present invention it will be clear for the skilled person if a reference to a protein, polypeptide, nucleic acid, or polynucleotide includes reference to an “isolated” protein, polypeptide, nucleic acid, or polynucleotide.

As used herein, a “modulator” refers to a compound that alters the activity of a target activity, for example the activity of a target protein. The modulator may be an inhibitor (antagonist) or an enhancer (agonist). The modulator may alter the activity by modulation of, for example, the enzymatic activity of a target protein, by modulation the interaction of the target protein with a further factor, such as a further protein, by modulation of the activity of a regulator of the target protein, and/or by modulating expression of the target protein.

As used herein, the term “ortholog”, with regard to a gene or protein, refers to the homologous gene or protein found in another species, which has the same function as the gene or protein, but (usually) diverged in sequence from the time point when the species harboring the genes diverged (i.e. the genes evolved from a common ancestor by speciation). Orthologs of the gene of the invention may thus be identified in other species based on both sequence comparisons (e.g. based on percentages sequence identity over the entire sequence or over specific domains) and functional analysis.

As used herein, the term “pharmaceutical composition” refers to a composition formulated in pharmaceutically acceptable or physiologically acceptable compositions for administration to a cell or subject. The compositions of the invention may be administered in combination with other agents as well, provided that the additional agents do not adversely affect the ability of the composition to deliver the intended therapy. The pharmaceutical composition often comprises, in addition to a pharmaceutical active agent, one or more pharmaceutical acceptable carriers (or excipients). The pharmaceutical compositions be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

As used herein the term “nucleic acid” or “polynucleotide” refers to any polymers or oligomers of (contiguous) nucleotides. The nucleic acid may be DNA or RNA, or a mixture thereof, and may exist permanently or transitionally in single-stranded or double-stranded form, including homoduplex, heteroduplex, and hybrid states. The present invention also contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogenous in composition, and may be isolated from naturally occurring sources or may be artificially or synthetically produced.

As used herein, “protein” or “polypeptide” are used interchangeably and refer to molecules consisting of a chain of amino acids, without reference to a specific mode of action, size, 3-dimensional structure or origin. A “fragment” or “portion” of a protein may thus still be referred to as a “protein.” A protein as defined herein and as used in any method as defined herein may be an isolated protein. An “isolated protein” is used to refer to a protein which is no longer in its natural environment, for example in vitro or in a recombinant host cell.

As used herein, “sequence”, “amino acid sequence” or “(poly) nucleotide sequence” refers to the order of amino acids, nucleotides of, or within a polypeptide or nucleic acid/polynucleotide. In other words, any order of amino acids or nucleotides may be referred to as a sequence (amino acids sequence, nucleotide sequence).

As used herein, a “subject” is to indicate an organism from which (cell) material may be obtained. The subject may be any subject in accordance with the present invention, including, but not limited to humans, males, females, infants, children, adolescents, adults, young adults, middle-aged adults or senior adults and/or other primates or mammals. Preferably the subject is a human patient. A subject may have been diagnosed with a disease, for example cancer, or be suspected of having such disease.

As used herein, the terms “construct”, “nucleic acid construct”, “vector”, and “expression vector” may be used interchangeably and are defined as a man-made nucleic acid molecule resulting from the use of recombinant DNA technology. These constructs and vectors therefore do not include naturally occurring nucleic acid molecules although a nucleic acid construct may comprise (parts of) naturally occurring nucleic acid molecules.

### DETAILED DESCRIPTION

### Summary of the Invention

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to the surprising identification of the TMEM68 protein (and orthologs), herein referred to as DIESL polypeptide, as a new triacylglycerol producing protein, or with triacylglycerol synthase activity. Next to DGAT1 and DGAT2, DIESL is, to the knowledge of the inventors, the third protein that has been recognized in humans that is able to produce triacylglycerol, albeit by a mechanism that is distinguishable from DGAT1 and DGAT2.

At the same time, the present invention is related to the surprising identification of a further protein, herein referred to as TMX1 polypeptide, that act as a natural regulator of DIESL and DIESL mediated TAG production.

As will be understood by a skilled person, the present invention is further directed to orthologs of the DIESL polynucleotide and/or polypeptide, as wells as to orthologs of the TMX1 polynucleotide and/or polypeptide, and/or to polypeptides and polynucleotides that share a certain level of (amino acid) identity with the DIESL polypeptide or with the TMX1 polypeptide, and, preferably, displaying the same or comparable activity, such as those disclosed herein (e.g. TAG synthase activity and inhibition of DIESL-mediated TAG accumulation and/or production).

The present invention allows for the use of DIESL and/or TMX1 in methods of producing or inhibiting production or accumulation of TAG. The present invention also allows for the screening of agent able to modulate DIESL polypeptide activity, TMX1 polypeptide activity and/or modulate TAG production and/or in screening for mutants of DIESL and TMX1 with altered activity.

Therefore, according to an aspect of the present invention there is provided for an isolated DIESL polypeptide, wherein the DIESL polypeptide comprises an amino acid sequence that has at least 75% amino acid sequence identity with the amino acid sequence as defined in SEQ ID NO:1, wherein the DIESL polypeptide exhibits triacylglycerol synthase activity. Preferably the polypeptide has 90% or higher sequence identity with the DIESL polypeptide of SEQ ID NO: 1. SEQ ID NO: 1 is the polypeptide sequence of (human) transmembrane protein 68. Also provided is for orthologs of the DIESL polypeptide, and that, preferably, like DIESL, exhibit triacylglycerol synthase activity.

According to an aspect of the present invention there is also provided for an isolated DIESL polynucleotide, as wells as orthologs thereof.

According to an aspect of the present invention there is also provided for a vector comprising the DIESL polypeptide.

According to an aspect of the present invention there is also provided for a host cell. The host cell comprises the DIESL polynucleotide, polypeptide and/or vector. In some embodiments, the cell is a brain cell, a cell from a metabolic tissue, a cell from adipose tissue, an adipocyte, a liver cell, a muscle cell, or a skeletal muscle cell. According to aspect of the present invention there is also provided for the use of the DIESL polypeptide, polynucleotide, vector, and/or DIESL orthologs in the production of triacylglycerol. In preferred embodiment the DIESL polypeptide exhibits triacylglycerol synthase activity as described herein. The DIESL polypeptide may be used in a cell free system, but preferably, is present in a cell. In some embodiments, the triacylglycerol produced is obtained and/or purified, at least to a certain degree.

According to another aspect there is provided for a screening method for identifying agents that may modulate activity, for example, triacylglycerol production, of the DIESL polypeptide. Such agent may, for example, be identified by comparison to a control situation, for example, wherein the agent is absent. In some embodiments, the screening method is in a cell free system. In other embodiments, the screening is performed using cells that expresses or comprises the DIESL polypeptide, polynucleotide and/or vector of the present invention. In some embodiments, modulating of the activity is determined and/or measured by detecting triacylglycerol synthase activity, triacylglycerol production, triacylglycerol accumulation, incorporation of a fatty acid into a diacylglycerol, and/or formation of lipid droplet organelles. In some embodiment, the cell that expresses the DIESL polypeptide does not express a further diglyceride acyltransferase, preferably does not express DGAT1, DGAT2 (or orthologs thereof), or both DGAT1 and DGAT2. In these embodiments, TAG production by other proteins than DIESL is reduced, or even abolished. In some embodiments the cell that expresses the DIESL polypeptide does not express TMX1 (or orthologs), preferably in addition to the cells not expressing, for example, DGAT1, DGAT2, or both. In these embodiments, regulation by TMX1 of TAG accumulation by DIESL is reduced, or even abolished. In some embodiments, the agent to be screened is a known modulator of a (further) diglyceride acyltransferase such as a known modulator, for example, inhibitor, of DGAT1 and/or DGAT2.

According to an aspect of the present invention there is provided for a screening method for identifying an agent that modulates the interaction between a DIESL polypeptide and a TMX1 polypeptide. As part of the present invention it was found that the DIESL polypeptide and the TMX1 polypeptide interact, and wherein the TMX1 polypeptide act as a (negative) regulator of DIESL activity. Agents that modulate interaction between DIESL and TMX1 may be identified by the ability of such compounds to, for example, modulate the (relative) presence of dimers formed by the interaction between the DIESL polypeptide and the TMX1 polypeptide, or the respective monomers.

According to an aspect of the invention there is also provided for a method for screening an agent that modulates activity of a (further) diglyceride acyltransferase (i.e. not DIESL), in particular DGAT1 and/or DGAT2, in the absence of DIESL polypeptide. Such methods allow for the identification of agents that modulate of DGAT1 and/or DGAT2 activity without the results being influenced by DIESL activity.

According to an aspect of the present invention there is also provided by a screening method to identify mutants of the DIESL polypeptide, wherein said mutant has altered activity and/or altered substrate specificity compared to a control, for example, compared to wildtype DIESL polypeptide. Such screening methods may include determining activity and/or substrate preference of mutant DIESL polypeptides.

According to an aspect of the present invention there is also provided for a method of producing a pharmaceutical composition wherein the method comprising the identification of an agent as disclosed herein and furthermore the mixing of the agent identified, or a derivate or homologue thereof, with a pharmaceutically acceptable carrier. The thus provided pharmaceutical composition finds utility in the treatment of various diseases and conditions that involve TAG.

According to an aspect of the present invention there is also provided for a method for producing TAG, comprising contacting the DIESL polypeptide with a diglyceride and a source of fatty acids. In some embodiments the DIESL polypeptide is expressed in a cell, e.g. a host cell.

According to an aspect of the present invention there is also provided for a method of determining TAG production and/or DIESL activity in a cell. Such method may comprise determining expression, amount, level, and/or activity of a DIESL polypeptide and/or polynucleotide, a TMX1 polypeptide and/or polynucleotide, and/or DIESL/TMX1 monomer and or dimer. In some embodiments, the cells are obtained from a subject, preferably a human subject. In some embodiments the cells are obtained from healthy or diseased tissue of said subject. In some embodiments, the cell is a brain cell, a cell from a metabolic tissue, a cell from adipose tissue, an adipocyte, a liver cell, a muscle cell, or a skeletal muscle cell.

According to an aspect of the present invention there is also provided for an isolated TMX1 polypeptide, or ortholog, having at least 75% amino acid sequence identity with the amino acid sequence of SEQ ID NO:2. In some, preferred, embodiments, the TMX1 polypeptide is able of regulating DIESL polypeptide mediated TAG production or accumulation, and/or DIESL triacylglycerol synthase activity. Also provided are an isolated TMX1 polynucleotide, a vector comprising such TMX1 polynucleotide, and/or a host cell comprising the TMX1 polypeptide, polynucleotide and/or vector.

According to an aspect of the present invention there is also provided for use of the TMX1 polypeptide, polynucleotide, and/or vector for inhibiting the production or accumulation of TAG, preferably for inhibiting DIESL mediated TAG production.

According to an aspect of the present invention there is also provided for a screening method for identifying mutants of TMX1 polypeptide, wherein said mutant has altered regulation of DIESL mediated triacylglycerol accumulation. Such screening method may comprise, contacting a DIESL polypeptide and a mutant TMX1 polypeptide, and detecting activity of a mutant TMX1 polypeptide compared to a control and/or detecting activity of the DIESL polypeptide compared to control to determine the mutant of the TMX1 polypeptide's altered inhibition of DIESL mediated triacylglycerol production or accumulation.

According to an aspect of the present invention there is also provided for use of an oligonucleotide to knock down or knock out DIESL and/or TMX1, for modulating TAG production in a cell or in an organism.

According to an aspect of the present invention there is also provided for a cell that is a non-animal cell that expresses a DIESL polypeptide and/or a TMX1 polypeptide; is a non-human cell that expresses human DIESL polypeptide and/or TMX1 polypeptide; is a cell that has been modified to not express a DIESL polypeptide and/or a TMX1 polypeptide, and that has been modified to not express a DGAT1 polypeptide; is a cell that has been modified to not express a DIESL polypeptide and/or a TMX1 polypeptide, and that has been modified to not express a DGAT2 polypeptide; is a cell that has been modified to not express a DIESL polypeptide and/or a TMX1 polypeptide, and that has been modified to not express a DGAT1 polypeptide and a DGAT2 polypeptide.

Finally, according to an aspect of the present invention there is also provided for a non-human transgenic knockout animal, in which the gene encoding for a DIESL polypeptide is knocked out. Orthologs of the gene encoding for the DIESL polypeptide, as well as for the expressed protein have been identified in several species.

In particular, the knocked out gene encodes for a DIESL polypeptide which polypeptide has at least 75% amino acid sequence identity with the amino acid sequence as defined in SEQ ID NO: 1 and that exhibits triacylglycerol synthase activity, is knocked out.

### DETAILED DESCRIPTION

### Polypeptides, Polynucleotides, Vectors

The present invention is based on the surprising identification of two proteins, herein referred to as DIESL and TMX1, as being involved in a hitherto unknown route of TAG production and accumulation.

Therefore, in various embodiments of the present invention, there is provided for DIESL polypeptides, TMX1 polypeptides, polynucleotides encoding such peptides, as well as vectors comprising such polynucleotides.

In some embodiment the polypeptides of the present invention are isolated polypeptides. For example, the polypeptide or protein is in an environment other than its native environment, such as apart from blood and native animal tissue. In some embodiments, the isolated polypeptide is substantially free of other polypeptides, particularly other polypeptides of animal origin. It may be preferred to provide the polypeptides in a highly purified form, i.e. greater than 95% pure, more preferably greater than 99%, pure. When used in this context, the term “isolated” does not exclude the presence of the same polypeptide in alternative physical forms, such as dimers or alternatively glycosylated or derivatized forms.

In particular there is provided for an isolated DIESL polypeptide, wherein the DIESL polypeptide comprises an amino acid sequence that has at least 75% amino acid sequence identity with the amino acid sequence as defined in SEQ ID NO:1, wherein the DIESL polypeptide exhibits triacylglycerol synthase activity.

In some embodiments, the DIESL polypeptide comprises an amino acid sequence having at least 90% sequence identity to SEQ ID NO: 1, having at least 95% sequence identity to SEQ ID NO: 1, having at least 98% sequence identity to SEQ ID NO: 1, having at least 99% sequence identity to SEQ ID NO: 1, having the amino acid sequence as defined in SEQ ID NO:1, or is transmembrane protein 68.

In other words, there is provided for an (isolated) DIESL polypeptide as defined above, and wherein the DIESL polypeptide exhibits TAG synthase activity, for example, in a manner as disclosed herein.

Likewise, there is provided for an isolated TMX1 polypeptide, wherein the TMX1 polypeptide comprises an amino acid sequence that has at least 75% amino acid sequence identity with the amino acid sequence as defined in SEQ ID NO:2, wherein the TMX1 polypeptide exhibits inhibition of DIESL mediated triacylglycerol synthase activity or accumulation.

In some embodiments, the TMX1 polypeptide comprises an amino acid sequence having at least 90% sequence identity to SEQ ID NO: 1, having at least 95% sequence identity to SEQ ID NO: 1, having at least 98% sequence identity to SEQ ID NO: 2, having at least 99% sequence identity to SEQ ID NO:2, having the amino acid sequence as defined in SEQ ID NO:2, or is TMX1.

In other words, there is provided for an (isolated) TMX1 polypeptide as defined above, and wherein the TMX1 polypeptide exhibits inhibition of DIESL mediated triacylglycerol accumulation, for example, in a manner as disclosed herein.

The DIESL polypeptide and the TMX1 polypeptide and their respective activities were identified in human cells (see Examples) and are represented by SEQ ID NO: 1 and SEQ ID NO:2, respectively. The skilled person understands that similar proteins (and genes encoding such proteins), e.g. orthologs (i.e. a polypeptide or protein obtained from one species that is the functional counterpart of a polypeptide or protein from a different species. Sequence differences among orthologs are the result of speciation) are likewise contemplated by the current invention. The skilled person knows how to identify such similar proteins, e.g. orthologs, in other species, for example in other mammals or in other animals such as rodents.

The present invention also provides for DIESL and/or TMX1 polypeptides that comprise an amino acid sequence that is substantially homologous to the polypeptides of SEQ ID NO:1 and SEQ ID NO: 2, respectively, and to their orthologs. The term “substantially homologous” is used herein to denote that the amino acid sequence comprised in the polypeptides having 75%, preferably 80%, 85%, more preferably at least 90%, sequence identity to the sequences shown in SEQ ID NO: 1 and 2 or their orthologs. Such polypeptides will comprise an amino acid sequence that more preferably be at least 95% identical, and most preferably 98% or more identical to SEQ ID NO: 1 or SEQ ID NO: 2, respectively, or its orthologs. Percent sequence identity is determined by conventional methods. In a preferred embodiment the amino acid sequence comprised in the polypeptide(s) of the present invention has 100% sequence identity to SEQ ID NO: 1, to SEQ ID NO:2, or to the orthologs thereof. In some embodiments, the DIESL polypeptide is (human) transmembrane protein 68, or an ortholog thereof. In some embodiment the TMX1 polypeptide is (human) TMX1 or an ortholog thereof.

Transmembrane protein 68, TMEM68 (Gene ID: 37695, see https://www.ncbi.nlm.nih.gov/gene/137695) has been described as an endoplasmic reticulum-anchored protein. The positioning of TMEM68 at the ER was dependent on its first transmembrane domain (TMD), which by itself was sufficient to target cytosolic green fluorescence protein (GFP) to the ER. In contrast, a second TMD was dispensable for ER localization of TMEM68. The authors concluded that TMEM68 may be part of a brain-specific set of enzymes implicated in neuronal glycerolipid homeostasis (Chang et al. PLoS One. 2017; 12 (5)). TMEM68 has also been implicated as a biomarker in feed intake and growth phenotypes in cattle (Lindholm-Perry et al. Anim Genet 2012 April; 43 (2): 216-9). However, no function for TMEM68 was proposed. In this study TMEM68 is expressed in bovine rumen, abomasum, intestine and adipose tissue. Thus wherein in the application reference is made to a an amino acid having a particular percentage of sequence identity to SEQ ID NO:1, in an embodiment, this refers to having a particular percentage of sequence identity to TMEM68 (Gene ID: 37695). The person skilled in the art understands how to determine if a particular amino acid sequence is to be recognized as a DIESL protein according to the invention (including orthologs and mutants thereof).

Thioredoxin related transmembrane protein 1, TMX1, (Gene ID: 81542, see https://www.ncbi.nlm.nih.gov/gene/81542) has been implicated in cancer cell metabolism as a thiol-based modulator of ER-mitochondria Ca2+ flux (Raturi et al. J Cell Biol. 2016 Aug. 15;214 (4): 433-44), and has been suggested to have a protective role in inflammatory liver injury (Matsuo et al. Antioxid Redox Signal (2013) April 10;18 (11): 1263-72). TMX1 has been described as a transmembrane oxidoreductase in the endoplasmic reticulum (ER). TMX1 structure and features has recently been reviewed by Guerra et al (Cells 2020 Aug. 31;9 (9): 2000). The TMX protein family comprises five membrane-tethered Protein Disulfide Isomerases (TMX1, TMX2, TMX3, TMX4 and TMX5) and these proteins are all characterized by an N-terminal signal sequence for ER targeting and one catalytically active thioredoxin (TRX)-like domain (known as type-a TRX-like domain), containing the active site. Protein Disulfide Isomerases like TMX1 are believed to regulate formation, isomerization and disassembly of covalent bonds between cysteine residues. Thus wherein in the application reference is made to a an amino acid having a particular percentage of sequence identity to SEQ ID NO: 2, in an embodiment, this refers to having a particular percentage of sequence identity to TMX1 (Gene ID: 81542). The person skilled in the art understands how to determine if a particular amino acid sequence is to be recognized as a TMX1 protein according to the invention (including orthologs and mutants thereof).

The following table 1 shows SEQ ID NO: 1 and SEQ ID NO: 2:

SEQ ID NO: 1
MIDKNQTCGVGQDSVPYMICLIHILEEWFGVEQLEDYLNFANYLL
WVFTPLILLILPYFTIFLLYLTIIFLHIYKRKNVLKEAYSHNLWDGAR
KTVATLWDGHAAVWHGYEVHGMEKIPEDGPALIIFYHGAIPIDFYY
FMAKIFIHKGRTCRVVADHFVFKIPGFSLLLDVFCALHGPREKCVE
ILRSGHLLAISPGGVREALISDETYNIVWGHRRGFAQVAIDAKVPII
PMFTQNIREGFRSLGGTRLFRWLYEKFRYPFAPMYGGFPVKLRT
YLGDPIPYDPQITAEELAEKTKNAVQALIDKHQRIPGNIMSALLERF
H
SEQ ID NO: 2
MAPSGSLAVPLAVLVLLLWGAPWTHGRRSNVRVITDENWRELLE
GDWMIEFYAPWCPACQNLQPEWESFAEWGEDLEVNIAKVDVTE
QPGLSGRFIITALPTIYHCKDGEFRRYQGPRTKKDFINFISDKEWK
SIEPVSSWFGPGSVLMSSMSALFQLSMWIRTCHNYFIEDLGLPV
WGSYTVFALATLFSGLLLGLCMIFVADCLCPSKRRRPQPYPYPSK
KLLSESAQPLKKVEEEQEADEEDVSEEEAESKEGTNKDFPQNAIR
QRSLGPSLATDKS

Also provided is for isolated polynucleotides, wherein the polynucleotide encodes for the polypeptides of the present invention. In some embodiments, the polynucleotide is an isolated polynucleotide. For example, the polynucleotide has been removed from its natural genetic environment and is thus free of other extraneous or unwanted coding sequences and is in a form suitable for use within genetically engineered protein production systems. Such isolated polynucleotides include those that are separated from their natural environment and include cDNA and genomic clones. Isolated DNA molecules of the present invention are free of other genes with which they are ordinarily associated but may include naturally occurring 5′ and 3′ untranslated regions such as promoters and terminators. He polynucleotides of the present invention includes both DNA and RNA and may be single-stranded or double-stranded. Methods for obtaining or producing such polynucleotides are well-known to the skilled person. The skilled person understands that, in view of the degeneracy of the genetic code, considerable sequence variation is possible among these polynucleotide molecules.

In particular, there is provided for a polynucleotide, wherein the polynucleotide encodes for the DIESL polypeptide as defined herein (e.g. including its orthologs).

In particular, there is provided for a polynucleotide, wherein the polynucleotide encodes for the TMX1 polypeptide as defined herein (e.g. including its orthologs).

Also provided is for vectors, e.g. expression vectors comprising the polynucleotides of the present invention. The skilled person is well aware of such vectors. Such vector is, for example, generally a DNA molecule, linear or circular, that comprises a segment encoding the polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA or may contain elements of both.

The vector may commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers

In particular there is provided for a vector comprising the DIESL polynucleotide as defined herein.

In particular there is provided for a vector comprising the TMX1 polynucleotide as defined herein.

In a preferred embodiment there is provided for an expression vector comprising the following operably linked elements: a transcription promoter; a polynucleotide molecule as defined herein, and a transcription terminator.

### Host Cells, Cells

As is disclosed herein, the polypeptides, polynucleotides, (expression) vectors of the present invention, may be comprised in a wide variety of different cells, including cells native to the polypeptides and/or polynucleotides and cells that are non-native to the polypeptides and/or polynucleotides. In one example, there may be provided for a polypeptide of the present invention and wherein said polypeptide has been modified relative to its native form, and wherein the modified polypeptide of the invention is expressed in a cell which is native to the unmodified polypeptide of the invention. In another example, a polypeptide of the present invention, isolated from its native cells, is expressed in a non-native cell.

It is also provided that the native or non-native cell has been further modified. For example, the cell wherein a polypeptide and/or polynucleotide of the present invention is comprised (or expressed) may have been modified, e.g. genetically modified, such that the expression of other genes/proteins in the cell has been modified, either temporarily or permanent. For example, one or more genes may have been modified so that they no longer express a (functional) protein. In another example, one or more genes may have been genetically modified such that expression one or more proteins is increased or decreased.

Suitable (host) cells are those cell types that can be transformed or transfected with exogenous DNA and grown in culture, and include bacteria, fungal cells, and cultured higher eukaryotic cells. Eukaryotic cells, particularly cultured cells of multicellular organisms, may be preferred. In some embodiments, the cell may also include cells that are obtained from a patient or from a diseased part of the patient (e.g. a tumor). Techniques for manipulating cloned DNA molecules and introducing exogenous DNA into a variety of host cells are well known to the skilled person.

For example, cultured mammalian cells are suitable hosts within the present invention. Other higher eukaryotic cells can also be used as hosts, including plant cells, insect cells and avian cells. Fungal cells, including yeast cells, can also be used within the present invention. Yeast species of particular interest in this regard include Saccharomyces cerevisiae. Prokaryotic host cells, including strains of the bacteria Escherichia coli, Bacillus and other genera are also useful host cells within the present invention.

In some embodiment, the cell or host cell used in the present invention is selected from those described herein. In particular there is provided for a host cell, such as those described herein, comprising the DIESL polypeptide, the DIESL polynucleotide and/or the vector. In particular there is also provided for a host cell, such as those described herein, comprising the TMX1 polypeptide, the TMX1 polynucleotide and/or the vector.

In certain embodiments, in particular in those embodiments wherein the DIESL polypeptide is used in the production of a triacyl glycerol (TAG), those embodiments that relates to methods of producing TAG, and those embodiments relating to the screening for compounds that may modulate the activity of the DIESL polypeptide of the invention, and wherein the DIESL polypeptide is, for example, comprised (expressed) in a cell, the cell is selected from the group consisting of those cell defined above, and in particular a prokaryotic cell, an eukaryotic cell, a bacterium, a yeast cell, a fungus cell, an animal cell, a plant cell, a cell that naturally lacks the ability to produce triacylglycerol, a cell that does not express a diglyceride acyltransferase, preferably a cell that does not express a Diacylglycerol O-acyltransferase 1 (DGAT1) polypeptide and/or a Diacylglycerol O-acyltransferase 2 (DGAT2) polypeptide, a cell wherein the expression of a DGAT1 polypeptide and/or a DGAT2 polypeptide is modified, a cell that does not express a Thioredoxin-related transmembrane protein 1 (TMX1) polypeptide, a cell wherein the expression of a TMX1 polypeptide is modified, a cell that does not express a DGAT1 polypeptide, a DGAT2 polypeptide, and/or a TMX1 polypeptide, or a cell that overexpresses the DIESL polypeptide.

A cell that naturally lacks the ability to produce triacylglycerol refers to any cell that does not comprise the cellular machinery to produce triacylglycerol from its precursors, even if such precursors would be available to the cell. Suitable examples of such cells include a wide variety of prokaryotes/bacteria. The presence of diverse types of TAG with different properties depending on their fatty acid composition is widespread among eukaryotic organisms such as yeast, fungi, plants and animals, whereas occurrence of TAG in bacteria has only rarely been described, with the possible exception of bacteria belonging to the actinomycetes group, such as species of Mycobacterium, Streptomyces, Rhodococcus and Nocardia. Another example contemplated are cells that may originally have the ability to produce TAG but that has been modified or has mutated and as the consequence thereof has lost its ability to produce TAG. It will be understood, as is shown in the Examples, that the introduction of a DIESL polypeptide of the present invention in such cell that lacks the ability to produce TAG may provide such cell with the ability to produce TAG. For example, introducing a DIESL polypeptide of the invention in bacteria may provide such bacteria with the ability to produce TAG, and introducing a DIESL polypeptide of the invention in a cell that was modified to lack the ability to produce TAG may lead to the restoration of TAG synthesis or production by such cell.

The cell wherein the DIESL polypeptide is comprised may also be a cell that does not express a diglyceride acyltransferase, i.e. that does not express another protein than the DIESL polypeptide of the present invention and which protein has diglyceride acyltransferase activity. In other words, such cell does not comprise an (other) enzyme that is able to produce TAG by diglyceride acyltransferase activity. For example, the cell does not express a Diacylglycerol O-acyltransferase 1 (DGAT1) polypeptide and/or a Diacylglycerol O-acyltransferase 2 (DGAT2) polypeptide.

The cell wherein the DIESL polypeptide is comprised may also be a cell wherein the expression of such DGAT1 polypeptide and/or a DGAT2 polypeptide is modified, for example increased or decreased. DGAT1 and DGAT2 are well-known examples of diglyceride acyltransferases (see, for example, Chitraju et al. J Lipid Res 2019 June; 60 (6): 1112-1120). DGAT1 and DGAT2 are evolutionarily unrelated acyl-CoA: diacylglycerol acyltransferase (DGAT) enzymes and which catalyze the same reaction. With the context of the present invention reference to DGAT1 and DGAT2 includes reference to the respective orthologs of these enzymes in other organisms.

The cell wherein the DIESL polypeptide is comprised may also be a cell that does not express a Thioredoxin-related transmembrane protein 1 (TMX1) polypeptide, or a cell wherein the expression of a TMX1 polypeptide is modified. As shown in the Examples, TMX1 modulates, in particular inhibits, DIESL mediated TAG accumulation. It is therefore in some embodiment of the present invention preferred that the cell wherein the DIESL polypeptide is comprised has no, modified, low or reduced expression of TMX1. With the context of the present invention reference to TMX1 includes reference to the orthologs of TMX1 in other organisms.

In some other embodiment of the present invention, the cell wherein the DIESL polypeptide is comprised is a cell that does not express a DGAT1 polypeptide, a DGAT2 polypeptide (or both), and/or a TMX1 polypeptide

In some other embodiment of the present invention, the cell wherein the DIESL polypeptide is comprised is a cell that is under stress conditions, in particular under ER stress conditions and/or under conditions of nutrient deprivation. Within the context of the current invention, a cell that is under stress conditions includes a cell that, although it expresses TMX1, shows dependency on DIESL activity as compared to situations wherein the stress condition is absent (for example, at least in part, requires and/or relies on DIESL activity as disclosed herein for functioning under such stress conditions). In some embodiments, the cell is a cell that has not been modified with respect to the gene(s) encoding TMX1 and/or DIESL, and, for example, is a cell that contain wild-type TMX1 and/or DIESL, and that is under stress conditions causing increased DIESL activity.

Finally, the cell wherein the DIESL polypeptide is comprised, including any of the cells described herein, is a cell that overexpresses the DIESL polypeptide.

With respect to those embodiments that relate to the screening of agents that may modulate the activity of the DIESL polypeptide of the invention, there is in particular provided that the cell wherein the DIESL polypeptide is comprised may be any cell (also referred to as host cell) as described herein. In some embodiments, the cell may also include cells that are obtained from a patient or from a diseased part of the patient (e.g. a tumor).

In some embodiments relating to the screening methods, the cell does not express a diglyceride acyltransferase, preferably does not express a DGAT1 polypeptide, a DGAT2 polypeptide and/or a DGAT1 polypeptide and a DGAT2 polypeptide; and/or does not express a TMX1 polypeptide.

In some embodiments, the cell, in any embodiment of the invention, is a brain cell, a cell from a metabolic tissue, a cell from adipose tissue, an adipocyte, a liver cell, a muscle cell, or a skeletal muscle cell.

According to another aspect of the current invention there is provided for particular cell. In particular there is provided for a cell that:—is a non-animal cell that expresses a DIESL polypeptide and/or a TMX1 polypeptide;—is a non-human cell that expresses human DIESL polypeptide and/or TMX1 polypeptide;—is a cell that has been modified to not express a DIESL polypeptide and/or a TMX1 polypeptide, and that has been modified to not express a DGAT1 polypeptide;—is a cell that has been modified to not express a DIESL polypeptide and/or a TMX1 polypeptide, and that has been modified to not express a DGAT2 polypeptide;—is a cell that has been modified to not express a DIESL polypeptide and/or a TMX1 polypeptide, and that has been modified to not express a DGAT1 polypeptide and a DGAT2 polypeptide. The skilled person knows, using conventional techniques, how to provide for such cells.

### Production of Triacylglycerol

As disclosed herein, the DIESL polypeptide of the present invention can suitable be used in the production of TAG. Therefor these is provided for the use of a DIESL polypeptide of the invention in the production of TAG. Likewise there is provided for a method for producing triacylglycerol, preferably wherein the method comprises contacting a DIESL polypeptide with a diglyceride (diacylglycerol) and a source of fatty acids under conditions sufficient for the triacylglycerol to be produced, preferably wherein the DIESL polypeptide is expressed in a cell.

In a preferred embodiment related to the production of TAG, the DIESL polypeptide exhibits triacylglycerol synthase activity.

Production of TAG with the DIESL polypeptide of the invention may be either in vitro or in vivo, but may also be performed extracellular, e.g. under conditions wherein the DIESL polypeptide and its substrates are in contact outside the context of a cell.

In case the DIESL polypeptide (or the DIESL polynucleotide encoding such DIESL polypeptide) is comprised in a cell, the cell (or host cell) may be any of the cells as described herein and above.

In certain embodiment the production of TAG is in in vitro, for example using cell culture systems including flasks and/or bioreactors, cell suspensions and so on. In certain embodiments the production of TAG is in vivo, for example wherein one, more or all cells of an organism, for example a plant or animal express a DIESL polypeptide according to the invention and thereby produces TAG. For example, genetically engineered animals, for example, cows, goats, sheep, or rodents like mice and rat, are contemplated, wherein these animals have been modified to (over) express a DIESL polypeptide of the invention, and/or has been modified to no longer or only to a very limited extent express a TMX1 polypeptide of the invention, such that the animals display modified TAG production and/or accumulation.

As already discussed herein elsewhere, in some embodiments, the cell used in the methods of producing TAG and comprising the DIESL polypeptide is selected from the group consisting off a prokaryotic cell, an eukaryotic cell, a bacterium, a yeast cell, a fungus cell, an animal cell, a plant cell, a cell that naturally lacks the ability to produce triacylglycerol, a cell that does not express a diglyceride acyltransferase, preferably a cell that does not express a Diacylglycerol O-acyltransferase 1 (DGAT1) polypeptide and/or a Diacylglycerol O-acyltransferase 2 (DGAT2) polypeptide, a cell wherein the expression of a DGAT1 polypeptide and/or a DGAT2 polypeptide is modified, a cell that does not express a Thioredoxin-related transmembrane protein 1 (TMX1) polypeptide, a cell wherein the expression of a TMX1 polypeptide is modified, a cell that does not express a DGAT1 polypeptide, a DGAT2 polypeptide, and/or a TMX1 polypeptide, or a cell that overexpresses the DIESL polypeptide.

In preferred embodiments of the use and methods relating to the production of TAG, the DIESL polypeptide is contacted with a diglyceride (or diacylglycerol) and a source of fatty acids under conditions sufficient for the triacylglycerol to be produced. The source of fatty acids may be any suitable source but is preferably selected from the group consisting of fatty acids, e.g. free fatty acids, an endogenous source of fatty acids (e.g. a source of fatty acids that is naturally present in the cell), or phospholipids. The fatty acid source may thus be a free fatty acid or fatty acid salt, but may also be a source and wherein the fatty acid is bound, e.g. by ester-linkage or by ether-linkage, to another molecule, for example in the form of a phospholipid, a lyso-phospholipid, a glycolipid, or the like. With respect to the fatty acids, it is contemplated that the source of fatty acids may also be provided externally. It is also contemplated that the fatty acids that are used as a source in the production of TAG by the DIESL polypeptide may have varying length, for example from C2 to C26, or longer, preferably from C4-C26, C6-C26, or C8-C26, C10-C26, or C12-C26, or longer. The fatty acids may be present in ester-lipids or in ether-lipids. The fatty acids may be saturated fatty acids or may be unsaturated fatty acids.

Also contemplated are the compositions produced by the use and methods relating to the production of TAG with the DIESL polypeptide of the invention. In preparing TAG, at least the direct substrates of the desired TAG, e.g. diglyceride (diacylglycerol) and fatty acid source, will be combined in the presence of the DIESL polypeptide under conditions sufficient for the acylation of the diglyceride (diacylglycerol) to occur.

As mentioned, of interest for use in producing TAG compositions are transgenic plants/fungi/animals that have been genetically manipulated using the polynucleotides of the invention, to produce triglycerides and/or compositions thereof in one or more desirable ways. Transgenic plants/fungi/animals of the present invention are those plants/fungi/animals that at least produce more TAG or TAG compositions than wild type, e. g. produce more oil, such as by producing seeds having a higher oil content, as compared to wild-type and/or produce triglyceride compositions, e. g. oils, that are enriched for triglycerides and/or enriched for one or more particular triglycerides as compared to wild type; and the like. Of particular interest are transgenic plants, such as canola, rapeseed, palm, oil, etc., which have been genetically modified to produce seeds having higher oil content than the content found in the corresponding wild type, where the oil content of the seeds produced by such plants is at least 10% higher, usually at least 20% higher, and in many embodiments at least 30% higher than that found in the wild type. The seeds produced by such DIESL transgenic plants can be used as sources of oil.

As already mentioned herein elsewhere, also of interest are transgenic non-human animals suitable for use as sources of food products and/or animal based industrial products. These trans-genic non-human animals, e. g. transgenic mice, rats, livestock, such as cows, pigs, horses, birds, etc., may be produced using methods known in the art and can be used for sources of a variety of different food and industrial products in which the triglyceride content is specifically tailored in a desirable manner. For example, transgenic animals that have been modified in a manner such that DIESL activity is modulated as compared to the wild type can be used as sources of food products that are low or high in triglyceride content, e. g. low or high fat meat products, low or high fat milk, low or high fat eggs, and the like.

The transgenic plants/fungi/animals described above can be readily produced by those of skill in the art with the polynucleotides and/or polypeptides as disclosed herein. In a preferred embodiment of the transgenic plants/fungi/animals described herein, these plants/fungi/animals have been modified to not express TMX1 (or ortholog thereof).

The triglyceride products obtained with the use and methods of the invention may be separated from the host cell using standard separation techniques.

In addition to the use of DIESL polypeptide in the production of TAG, there is also provided for the use of a TMX1 polypeptide in inhibiting the production of triacylglycerol, preferably in inhibiting DIESL polypeptide mediated triacylglycerol production. For example, the use or method may include introducing the TMX1 polypeptide in a system wherein TAG production, in particular DIESL polypeptide mediated TAG production should be inhibited or decreased. Therefore in some embodiment, the use of TMX1 polypeptide in inhibiting TAG production or accumulation, in particular in inhibiting DIESL-mediated TAG production is a use or method wherein the TMX1 polypeptide and/or the a TMX1 polynucleotide that encodes the TMX1 polypeptide is comprised in a cell that further comprises a DIESL polypeptide and/or a DIESL polynucleotide that encodes the DIESL polypeptide.

### Screening Assays-Agent

With the present invention disclosing for the first time that the DIESL polypeptide has TAG synthase activity, i.e. is involved in the production of TAG, and with the present invention disclosing for the first time that TMX1 is able to modulate, in particular inhibit, DIESL polypeptide mediated TAG accumulation, in particular by interaction with the DIESL polypeptide (see Examples), there is also provided for various screening assays. These screening assay are assays, for example, for identifying an agent that modulates the activity of a DIESL polypeptide, for identifying an agent that modulates interaction of a DIESL polypeptide with a TMX1 polypeptide, for identifying an agent that modulates the activity of an (other) diglyceride acyltransferase, preferably of DGAT1 and/or DGAT2, for identifying mutants of a DIESL polypeptide, wherein said mutant has altered activity and/or altered substrate preference, and for identifying mutants of a TMX1 polypeptide, wherein said mutant has altered inhibition of DIESL mediated triacylglycerol accumulation.

Based on the disclosure herein the skilled person will understand how to provide for suitable formats for such screening methods and for suitable conditions under which to perform such screening methods, for example as will be detailed herein. The screening method may be an in vitro or in vivo format, where both formats can readily be developed by those of skill in the art.

The candidate agents that may be tested in the various screening assays disclosed herein may be any type of molecule or mixture of molecules. The candidate agents encompass numerous chemical classes, including organic molecules, preferably small organic compounds having a molecular weight of more than 50 and less than about 2,500 daltons, antibodies, DNA and RNA molecules, other biomolecules such as peptides, saccharides, fatty acids, steroids, purines, pyrimidines, (oligo) nucleotides, know pharmaceutical drugs, derivatives, structural analogs or combinations thereof. The candidate agents may be obtained from a wide variety of sources including libraries of synthetic or natural compounds. Also contemplated are libraries of natural compounds in the form of bacterial, fungal, plant and animal extracts or natural or synthetically produced libraries and compounds, for example modified through conventional chemical, physical and biochemical means.

Compounds identified with the screening assays of the invention may find use in varies fields, included, but not limited to use in the treatment of health conditions that are related to TAG production, such as, for example, obesity, inflammation and cancer.

Using the screening methods described herein, a variety of different agents may be identified. Such agents may target the DIESL polypeptide and/or TMX1 polypeptide itself, or an expression regulator factor thereof. Such agents may inhibitors or promoters of DIESL polypeptide activity or TMX1 polypeptide activity, where inhibitors are those agents that result in at least a reduction of DIESL polypeptide activity or TMX1 polypeptide activity, respectively, as compared to a control and enhancers result in at least an increase in, respectively, DIESL polypeptide activity or TMX1 polypeptide activity, as compared to a control. Such agents may be used in a variety of therapeutic applications.

In some embodiment, the screening assays as disclosed herein are, screening assays designed to find modulatory agents of DIESL polypeptide activity, e.g. inhibitors or enhancers of DIESL polypeptide activity. Such agents may find use in a variety of applications, including as therapeutic agents, as agricultural chemicals, etc. The screening methods will typically be assays which provide for qualitative/quantitative measurements of DIESL polypeptide activity in the presence of a particular candidate therapeutic agent.

In particular, in some embodiments there is provided for an, preferably in vitro, screening method for identifying an agent that modulates the activity of a DIESL polypeptide, wherein the method comprises (a) contacting the DIESL polypeptide with a candidate agent; and (b) detecting a change in activity of the DIESL polypeptide compared to a control to determine the candidate agent's modulatory activity.

The screening assay may be performed in a cell free system, but preferably is performed under conditions wherein the DIESL polypeptide and/or the DIESL polynucleotide is comprised (or expressed) in a cell, such as those described herein elsewhere.

Modulation of the DIESL polypeptide activity may be measured using any suitable means. However, in some preferred embodiments DIESL polypeptide activity or a change in DIESL polypeptide activity is measured by detecting triacylglycerol synthase activity, triacylglycerol production, triacylglycerol accumulation, incorporation of a fatty acid into a diacylglycerol, and/or formation of lipid droplet organelles (see also the Examples). The skilled person understands how to detect triacylglycerol synthase activity, triacylglycerol production, triacylglycerol accumulation, incorporation of a fatty acid into a diacylglycerol, and/or formation of lipid droplet organelles using conventional techniques available in the art.

In some embodiments, the screening assay for identifying an agent that modulates the activity of a DIESL polypeptide comprises (a) providing a cell that expresses the DIESL polypeptide and providing a candidate agent; (b) contacting the candidate agent with the cell that expresses the DIESL polypeptide under conditions that allow the cell to produce triacylglycerol, in particular under conditions that allow the DIESL polypeptide to produce TAG; (c) comparing the activity of the DIESL polypeptide, preferably comparing triacylglycerol synthase activity, triacylglycerol production, triacylglycerol accumulation, incorporation of a fatty acid into a diacylglycerol, and/or formation of lipid droplet organelles to a control; and (d) identifying agents that provide for an increase or decrease in the activity of the DIESL polypeptide, preferably in triacylglycerol synthase activity, triacylglycerol production, triacylglycerol accumulation, incorporation of a fatty acid into a diacylglycerol, and/or formation of lipid droplet organelles. In some embodiments the method may also include identifying agent that do not provide for an increase or decrease in the activity of the DIESL polypeptide, for example in case it is desired that an agent with a known other biological activity does not modulate TAG production, in particular TAG production by a DIESL polypeptide. For example, it may be desirable that an agent with known inhibitory activity on DGAT1 and/or DGAT2 polypeptide activity does or does not modulate DIESL polypeptide activity (i.e. TAG production via DIESL polypeptide). Therefore, in some embodiments the candidate agent used in any of the screening assay or method described herein is a modulator of a diglyceride acyltransferase, preferably a DGAT1 polypeptide, a DGAT2 polypeptide and/or a DGAT1 polypeptide and a DGAT2 polypeptide.

In some embodiments, and as already discussed herein elsewhere, the screening method assay for identifying an agent that modulates the activity of a DIESL polypeptide makes use of a cell, wherein the cell does not express a (further) diglyceride acyltransferase, preferably does not express a DGAT1 polypeptide, a DGAT2 polypeptide and/or a DGAT1 polypeptide and a DGAT2 polypeptide; and/or does not express a TMX1 polypeptide. The skilled person knows how to provide for such cells using conventional techniques available in the prior art.

In some embodiment, the screening assay is an, preferably in vitro, screening method for identifying an agent that modulates interaction of a DIESL polypeptide with a TMX1 polypeptide. The skilled person knows how to provide for a suitable platform and suitable conditions for performing such screening method, using conventional techniques available in the prior art. In some embodiments, the screening method for identifying an agent that modulates interaction of a DIESL polypeptide with a TMX1 polypeptide, comprises (a) providing a cell that expresses the DIESL polypeptide and the TMX1 polypeptide; (b) providing a candidate agent; (c) contacting the candidate agent with the cell that expresses the DIESL polypeptide and the TMX1 polypeptide under conditions that allow interaction of the candidate agent with the DIESL polypeptide and/or the TMX1 polypeptide; (d) determining the amount of DIESL monomer, TMX1 monomer and/or the amount of DIESL/TMX1 dimer; (e) comparing the amounts obtained in step (d) to a control to determine the candidate agent's modulatory activity. The skilled person may determine the amount of DIESL monomer, TMX1 monomer and/or the amount of DIESL/TMX1 dimer using any conventional technique available in the prior art. By comparing the amount of DIESL monomer, TMX1 monomer and/or the amount of DIESL/TMX1 dimer to a control situation it can be determined if a candidate agent can modulate the interaction between the DIESL polypeptide and the TMX1 polypeptide, and thereby modulate DIESL polypeptide activity and/or modulate TMX1 polypeptide activity. For example, a compound identified to inhibit the formation of DIESL/TMX1 dimer, or identified to promote the dissolution of a DIESL/TMX1 dimer may be expected to allow improved TAG synthesis by the DIESL polypeptide and may be considered as an inhibitor of the inhibitory activity of the TMX1 polypeptide on DIESL polypeptide mediated TAG production.

In some embodiments, the screening method for identifying an agent that modulates interaction of a DIESL polypeptide with a TMX1 polypeptide comprises that the amount of DIESL/TMX1 dimer is determined by crosslinking the cells with, for example, PFA (formaldehyde/paraformaldehyde), lysing the cells and determine the amount of crosslinked dimer formed; lysing the cells, immunoprecipitate using an antibody binding for DIESL or TMX1 and determine the presence of the amount of TMX1 or DIESL, respectively in the immunoprecipitate; and/or lysing the cells, immunoprecipitate using an antibody binding for DIESL or TMX1 and determine the presence of the amount of TMX1 and DIESL in the immunoprecipitate.

As the skilled person understood, the present invention also allows for an improved screening method for identifying an agent that modulates the activity of an (other) diglyceride acyltransferase (i.e, wherein the diglyceride acyltransferase is not a DIESL polypeptide of the present invention) by including in the assay the agent may also be an agent that may modulate DIESL polypeptide activity. With such screening methods is now for the first time possible to establish whether an agent that modulates the activity of a non-DIESL polypeptide diglyceride acyltransferase is specific for that diglyceride acyltransferase or may, in addition, also modulate (inhibit or stimulate) the activity of a DIESL polypeptide. Therefore, in some embodiments there is provided for a, preferably in vitro, screening method for identifying an agent that modulates the activity of a diglyceride acyltransferase, preferably of DGAT1 and/or DGAT2, wherein the method comprises (a) contacting the diglyceride acyltransferase with a candidate agent; and (b) detecting a change in activity of the diglyceride acyltransferase compared to a control to determine the candidate agent's modulatory activity on the diglyceride acyltransferase, and wherein—the diglyceride acyltransferase is expressed in a cell that does not express a DIESL polypeptide; and/or—wherein the method further comprises performing the screening method to determine the candidate agent's modulatory activity on a DIESL polypeptide.

Likewise, in some embodiments the candidate agent used in any of the screening assay or method described herein is a modulator of DIESL polypeptide activity, and is screened for its modulation activity on other diglyceride acyltransferases, preferably a DGAT1 polypeptide, a DGAT2 polypeptide and/or a DGAT1 polypeptide and a DGAT2 polypeptide, for example in order to identify a modulator of DIESL polypeptide activity that is specific for said DIESL polypeptide and does not, or only to a limited extend, modulate the activity of another diglyceride acyltransferase such as DGAT1 and/or DGAT2 (and orthologs thereof).

In addition to providing screening methods for identifying agent that may modulate the activity of a DIESL polypeptide, a TMX1 polypeptide and/or a (further) diglyceride acyltransferase, such as DGAT1 and/or DGAT2, there is also provided for screening methods to identify mutants of a DIESL polypeptide and/or of a TMX1 polypeptide.

In some embodiment, the screening method is for identifying mutants of a DIESL polypeptide, wherein said mutant has altered activity and/or altered substrate preference. In some embodiments, the, preferably in vitro, method comprises (a) providing a mutant of the DIESL polypeptide; and (b) detecting activity of the mutant of the DIESL polypeptide compared to a control and/or detecting substrate preference of the mutant of the DIESL polypeptide compared to a control to determine the mutant of the DIESL polypeptide's altered activity and/or substrate preference. The skilled person knows, for example, using conventional techniques in the prior art how to prepare and provide for a mutant of a DIESL polypeptide.

For example, variants of the DIESL polypeptide may be provided by modification of the DNA encoding for a DIESL polypeptide, for example by insertion, deletion or substitution of one or more nucleotides, for example using conventional techniques including random mutagenesis and/or targeted mutagenesis including techniques such as CRISPR/CAS and the like. The skilled person also knows how to detect activity of the mutant of the DIESL polypeptide compared to a control (for example as described herein) and/or how to detect substrate preference (e.g, wherein the substrate is a source of fatty acids used by the DIELS polypeptide in the production of TAG) of the mutant of the DIESL polypeptide compared to a control, and in that way to determine the mutant of the DIESL polypeptide's altered activity and/or substrate preference.

In some embodiment, the, preferably in vitro, screening method is for identifying mutants of a TMX1 polypeptide, wherein said mutant has altered inhibition of DIESL mediated triacylglycerol accumulation, wherein the method comprises (a) providing a mutant of theTMX1 polypeptide and providing a DIESL polypeptide; (b) allowing the mutant TMX1 polypeptide to contact the DIESL polypeptide, preferably under conditions that allow the DIESL polypeptide to produce triacylglycerol; and (c) detecting activity of the mutant TMX1 polypeptide compared to a control and/or detecting activity of the DIESL polypeptide compared to control to determine the mutant of the TMX1 polypeptide's altered inhibition of DIESL mediated triacylglycerol accumulation. Again, the skilled person knows, for example, using conventional techniques in the prior art how to prepare and provide for a mutant of a TMX1 polypeptide, as well as how to detect activity of the mutant TMX1 polypeptide compared to a control and/or detecting activity of the DIESL polypeptide compared to control to determine the mutant of the TMX1 polypeptide's altered inhibition of DIESL mediated triacylglycerol synthase activity, for example, as described herein elsewhere.

### Pharmaceutical Compositions

Compounds identified with the screening assays of the invention may find use in varies fields, included, but not limited to use in the treatment of health conditions that are related to TAG production, such as, for example, obesity, inflammation and cancer. Therefor there is also provided for a method for producing a pharmaceutical composition comprising a screening method as disclosed herein, in particular a screening method for identifying an agent as described herein, and furthermore mixing the agent identified, or a derivative or homologue thereof, with a pharmaceutically acceptable carrier. In other words, there is also provided a method for producing a pharmaceutical composition comprising:
a screening method for identifying an agent that modulates the activity of a DIESL polypeptide, of a further diglyceride acyltransferase, or for identifying an agent that modulates interaction of the DIESL polypeptide with a TMX1 polypeptide according to any of the aspects and embodiments above;
selecting the identified agent that modulates the activity or the interaction; and
furthermore mixing the selected agent identified, or a derivative or homologue thereof, with a pharmaceutically acceptable carrier.

Thus, the identified agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. For oral preparations, the agents can be used alone or in combination with appropriate additives to make tablets, powders, granules or capsules, for example, with conventional additives. The agents can be formulated into preparations for injection by dissolving, suspending or emulsifying them in an aqueous or nonaqueous solvent. The agents can be utilized in aerosol formulations to be administered via inhalation. Furthermore, the agents can be made into suppositories by mixing with a variety of bases such as emulsifying bases or water-soluble bases.

The pharmaceutically acceptable excipients, such as vehicles, adjuvants, carriers or diluents, are available in the art. Moreover, pharmaceutically acceptable auxiliary substances, such as pH adjusting and buffering agents, tonicity adjusting agents, stabilizers, wetting agents and the like, are also available in the art.

The agents may find use in the treatment of a variety of different disease conditions involving TAG production and/or diacylglycerol metabolism, and particularly DIESL polypeptide activity, including both insufficient or hypo-DIESL activity and hyper-DIESL activity.

Representative diseases that may be treated include hyperlipidemia, cardiovascular disease, obesity, diabetes, cancer, neurological disorders, immunological disorders, and skin disorders associated with sebaceous gland activity, such as acne.

### Method for Measuring Triacylglycerol Production

Also provided is for methods for measuring TAG production. In some embodiments, such method comprises determining triacylglycerol production and/or DIESL activity in a cell wherein the method comprises determining DIESL polypeptide and/or TMX1 polypeptide expression, DIESL polypeptide and/or TMX1 polypeptide level, amount of DIESL/TMX1 dimer, and/or DIESL polypeptide and/or TMX1 polypeptide activity in the cell. The skilled person knows, for example, using conventional techniques in the prior art how to determine DIESL polypeptide and/or TMX1 polypeptide expression, DIESL polypeptide and/or TMX1 polypeptide level, amount of DIESL/TMX1 dimer, and/or DIESL polypeptide and/or TMX1 polypeptide activity in the cell. Based on the disclosure herein, it will be understood that an increase of DIESL polypeptide expression, level or amount or DIESL polypeptide activity are all indicative of the likeliness of increased TAG synthesis in the cell. As such, such increases can be taken to be an indication of increased TAG production in such cell (or decrease in case DIESL polypeptide expression, level or amount or DIESL polypeptide activity is decreased). Likewise, it will be understood that a decrease in TMX1 polypeptide expression, level or amount or TMX1 polypeptide activity are all indicative of the likeliness of increased TAG synthesis in the cell. As such, such decreases can be taken to be an indication of increased TAG production in such cell (or decreased in case TMX1 polypeptide expression, level or amount or DIESL polypeptide activity is increased). One way to determine triacylglycerol production and/or DIESL activity in a cell comprises determining TAG production, for example using a method as described herein, including detecting triacylglycerol synthase activity, triacylglycerol production, triacylglycerol accumulation, incorporation of a fatty acid into a diacylglycerol, and/or formation of lipid droplet organelles.

Although triacylglycerol production and/or DIESL activity may be determined in any suitable cell, is some embodiment, the cell is a human cell, wherein the cells is obtained from a subject, preferably a human subject, wherein the cell is obtained from healthy or diseased tissue, and/or wherein the cell is obtained from a tumor.

### Knock Down or Out

Also provided is for the use of an oligonucleotide to knock down or knock out DIESL and/or TMX1 for modulating triglyceride production in a cell or organism, preferably wherein the use comprises CRISPR/Cas technology, RNAi or antisense technology. Also provided is for a method for modulating triglyceride production in a cell or organism wherein the method comprises the use of an oligonucleotide to knock down or knock out DIESL and/or TMX1, preferably wherein the use comprises CRISPR/Cas technology, RNAi or antisense technology.

The skilled person knows, using conventional techniques in the prior art, how to design and use an oligonucleotide to knock down or knock out a DIESL polypeptide and/or a TMX1 polypeptide, and thereby modulate TAG production by the cell wherein the DIESL polypeptide and/or TMX1 polypeptide is knocked-down or knocked-out.

It will be understood that all details, embodiments and preferences discussed with respect to one aspect of embodiment as disclosed herein is likewise applicable to any other aspect or embodiment as disclosed herein and that there is therefore not need to detail all such details, embodiments and preferences for all aspect separately.

### Knock Down or Out Mice

Also provided is for a non-human transgenic knock out animal, in which the gene encoding for a DIESL polypeptide is knocked out.

The skilled person will understand for “knocked out” or “knocked down” (used both as synonymous), as a genetic technique in which one of an organism's genes is made inoperative (“knocked out” of the organism), or also indicated as silenced. Knockouts are accomplished through a variety of conventional techniques, including for example the homologous recombination (i.e., is carried out mainly by replacing an existing gene or disrupting it with an artificial piece of DNA), site-specific nucleases (e.g. zinc fingers coupled to endonucleases), transcription activator-like effector nuclease, and CRISPR/Cas technology.

In some embodiments of the non-human transgenic knockout animal, the knocked out gene encodes for a DIESL polypeptide which polypeptide has at least 75% amino acid sequence identity with the amino acid sequence as defined in SEQ ID NO: 1 and that exhibits triacylglycerol synthase activity.

In some embodiments, the non-human transgenic knockout animal is that wherein the knockout gene encodes for a DIESL polypeptide that comprises an amino acid sequence having at least 90% sequence identity to SEQ ID NO: 1, having at least from 91% to 95% sequence identity to SEQ ID NO: 1, or is transmembrane protein 68.

In some embodiments the non-humans transgenic knockout animal is a mammal, in particular is a rodent, more in particular selected from mice, rats, squirrels, prairie dogs, porcupines, beavers, guinea pigs, and hamsters.

In a more particular embodiment, the non-human transgenic knockout animal is a mouse.

This mouse of the previous embodiment is, in particular, a mouse in which the knocked out gene that encodes the DIESL polypeptide is the gene with accession number Gene ID 72098, of the National Center for Biotechnology Information database release of 26 Sep. 2022 (https://www.ncbi.nlm.nih.gov/gene/72098). This gene, whose transcript and protein correspond, respectively, to the accession number NM_028097.4 and NP_082373.1 in the same database and release, encodes for the mouse Transmembrane protein 68, TMEM68, ortholog of the human gene previously disclosed (i.e., Gene ID: 37695, see https://www.ncbi.nlm.nih.gov/gene/137695). The human and mouse TMEM68 proteins have a percentage of identity of 92.90%, in a pair-wise BLASTP alignments with 100% of sequence coverage.

Also herewith is provided for a method producing a non-human transgenic knockout animal, in particular a mouse, as defined in the previous aspect and embodiments, in which the gene encoding for a DIESL polypeptide, in particular for a mouse DIESL polypeptide, is functionally silenced by a single disruptive allele in the said gene (i.e., ortholog Diesl/Tmem68 gene).

In some embodiments, the non-human transgenic knockout animal is a mouse whose cells comprise a mitochondrial nicotinamide nucleotide transhydrogenase (NNT) that comprises a polypeptide sequence that has at least 99% amino acid sequence identity, or 100% amino acid sequence identity, with the amino acid sequence defined by the protein accession number NP_032736.2 of the National Center for Biotechnology Information database release of 12 Aug. 2022. The protein as defined in NP_032736.2 is the NAD (P) transhydrogenase, mitochondrial isoform 1 precursor of Mus musculus. It has 1086 amino acids. There is another isoform, which is the NAD (P) transhydrogenase, mitochondrial isoform 2 (NCBI accession number NM_001308506.1, NP_001295435.1, database release of 14-August-2022), which is a variant transcribed from this gene locus in C57BL/6 mice, which lacks five in-frame internal exons, compared to transcribed variant 1. The encoded isoform (2) is shorter (i.e., 835 amino acids) compared to isoform 1. Both mitochondrial isoforms result from gene identified with the accession number Gene ID: 18115, of the National Center for Biotechnology Information, database release of 26-Sep-2022.

This particular mouse expressing the mitochondrial nicotinamide nucleotide transhydrogenase (NNT) isoform 1 defined by the protein accession number NP_032736.2 explained above, has a phenotype with glucose tolerance and/or mithochondrial redox normal functioning in relation to a mouse expressing the isoform 2 of the mitochondrial NNT (i.e., NP_001295435.1). This animal expressing the long isoform of NNT protein (the one defined by NP_032736.2) is also herewith referred as the wild-type NNT (WT NNT).

In a more particular embodiment, the knockout mouse is a C57BL/6N mouse. This mouse expresses the mitochondrial nicotinamide nucleotide transhydrogenase (NNT) defined by the protein accession number NP_032736.2.

Of note is that, as will be illustrated in the examples below, assays carried out with this new knockout animal model allowed determining the surprising role of DIESL polypeptide under stress cell conditions, such as under ER stress, or under starving conditions. These non-previously suggested roles of the DIESL polypeptide could not have been clearly determined with another (animal) model, and makes DIESL an interesting key target for myriad of therapies (e.g., cancer, cachexia associated or not with cancer, feed disorders, nutrient deprivation or imbalance, etc., as examples of stress cell conditions).

Therefore, the herewith provided particular mouse knockout for the gene that encodes for a DIESL polypeptide (i.e., silenced diesl gene) but that preferably expresses the non-mutated mitochondrial nicotinamide nucleotide transhydrogenase, (NNT), for example, defined by the protein accession number NP_032736.2, supposes an improved and fair model for the analysis of the activity of DIESL polypeptide. Effectively, data observed with this model will not be influenced by any other pathway impaired due to mutated proteins, in particular mutated NNT.

### Assays Under Cell Stress Conditions (ER Stress and Starvation)

Derived from the surprising activity of DIESL under stress cell conditions, or of its role in the control or modulation of cell stress, also herewith provided are the screening methods disclosed in previous sections, in which when these methods are carried out with cells (wild-type or modified) or with animals, these cells or animals are under stress conditions, in particular under ER stress conditions or under nutrient deprivation.

Thus, some particular embodiments of screening methods for identifying an agent that modulates the activity of a DIESL polypeptide; and/or of screening methods for identifying an agent that modulates interaction of a DIESL polypeptide with a TMX1 polypeptide; and/or of screening methods for identifying mutants of a DIESL polypeptide, wherein said mutant has altered activity and/or altered substrate preference; and/or of methods for producing triacylglycerol, wherein the method comprises contacting a DIESL polypeptide expressed in a cell with a diglyceride and a source of fatty acids under conditions sufficient for the triacylglycerol to be produced; and/or of methods for determining triacylglycerol production and/or DIESL activity in a cell wherein the method comprises determining DIESL polypeptide and/or TMX1 polypeptide expression; the methods are carried out in cells or host cells comprising/expressing the DIESL polypeptide, and which cells are under cell stress conditions, in particular ER stress conditions and/or starvation conditions.

Also herewith provided is a particular embodiment of a cell that:
is a non-animal cell that expresses a DIESL polypeptide and/or a TMX1 polypeptide;
is a non-human cell that expresses human DIESL polypeptide and/or TMX1 polypeptide;
is a cell that has been modified to not express a DIESL polypeptide and/or a TMX1 polypeptide, and that has been modified to not express a DGAT1 polypeptide;
is a cell that has been modified to not express a DIESL polypeptide and/or a TMX1 polypeptide, and that has been modified to not express a DGAT2 polypeptide;
is a cell that has been modified to not express a DIESL polypeptide and/or a TMX1 polypeptide, and that has been modified to not express a DGAT1 polypeptide and a DGAT2 polypeptide; which cell has a phenotype of cell stress conditions, in particular ER stress conditions and/or starvation conditions.

The skilled person in the art will be able to identify markers or tests for cell stress conditions. “ER stress conditions” can be identified, for example, by determining the levels of certain transcription factors, such as the C/EBP homologous protein (CHOP), which is ubiquitously expressed at low levels under normal physiologic conditions, but that it is raised under ER stress conditions. Effects of starvation (i.e., deprivation of nutrients) on cells can be determined by the analysis of mitochondrial membrane potential and ATP levels, by the analysis of activation of AMP kinase, or in assays carried out with animals, by determining the body weight.

Having now generally described the invention, the same will be more readily understood through reference to the following examples which is provided by way of illustration and is not intended to be limiting of the present invention.

### EXAMPLES

### Results and Discussion

Triacylglycerols (TAGs), implicated in obesity and cardiovascular disease, constitute the primary unit of stored energy in the body, fueling mitochondrial beta-oxidation. In humans, TAGs are synthesized from excess fatty acyl-CoAs by diacylglycerol O-acyltransferases (DGAT1/DGAT2) yet alternative routes exist in distant eukaryotes. We disrupt the DGAT pathway in haploid human cells and use iterative genetics to uncover a TAG-synthesizing complex composed of DIESL (TMEM68, a protein of unknown function)—which we establish as specific TAG synthase—and its regulator TMX1. Whereas DIESL contributes to the cellular TAG pool in various cell lines, TMX1 loss leads to unbridled DIESL activity and lipid droplet formation. Mechanistically, TMX1 binds and controls DIESL. Thus, we identify an alternative TAG biosynthesis pathway driven by DIESL under potent control by TMX1.

Triacylglycerols (TAGs) are neutral lipids composed of a glycerol backbone conjugated to three fatty acyl (FA) chains and serve as the predominant unit of stored energy in a range of organisms, spanning (oleaginous) bacteria, algae, and mammals. In humans, most (if not all) cell types are able to synthesize triglycerides, and high levels of TAGs (hypertriglyceridemia) can lead to obesity and metabolic syndrome, whereas low levels contribute to cachexia, a multiorgan wasting disorder (1-5). Within cells, TAGs can be stored in lipid droplets, dedicated organelles providing energy to mitochondria through direct interorganellar contact sites (6). TAG synthesis in humans is carried out by diacylglycerol O-acyltransferase (DGAT) enzymes, which catalyze the CoA-dependent acylation of diacylglycerol produced by the Kennedy pathway (4, 7-10). DGAT1 and DGAT2 reside at the endoplasmic reticulum and are considered therapeutic targets for metabolic disease (11-14). DGAT-dependent TAG formation is typically limited by the availability of free fatty acids (4, 8, 15).

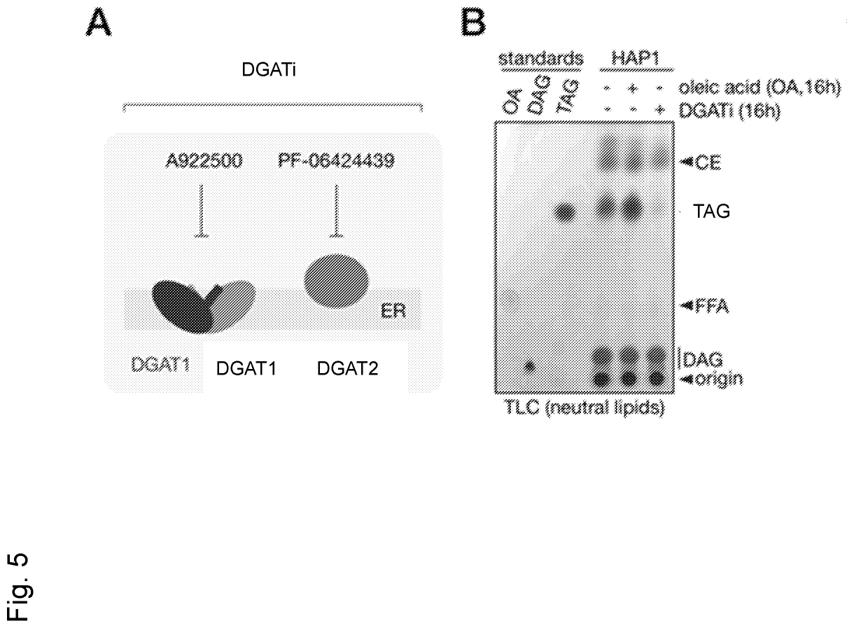

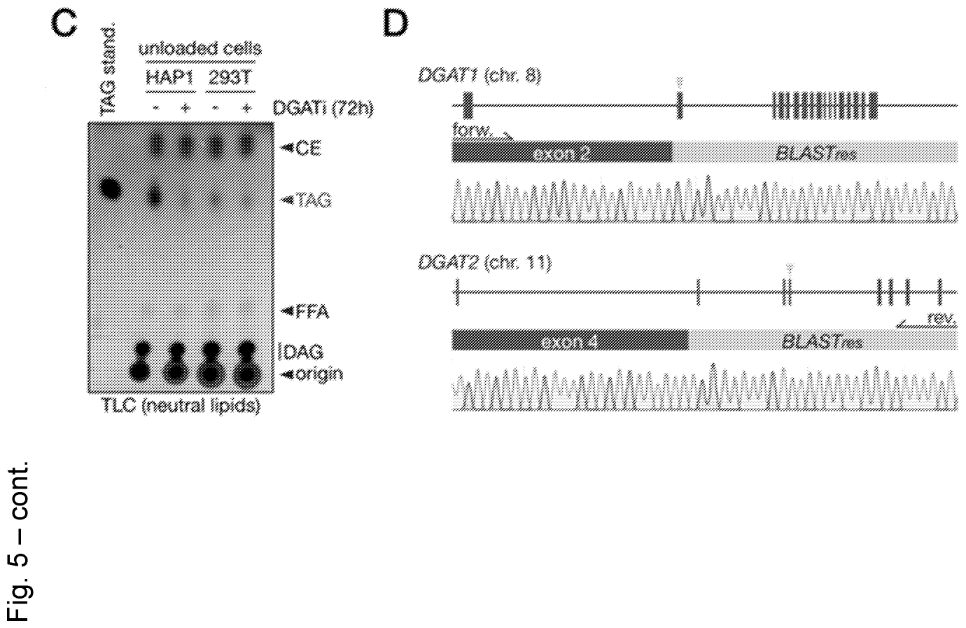

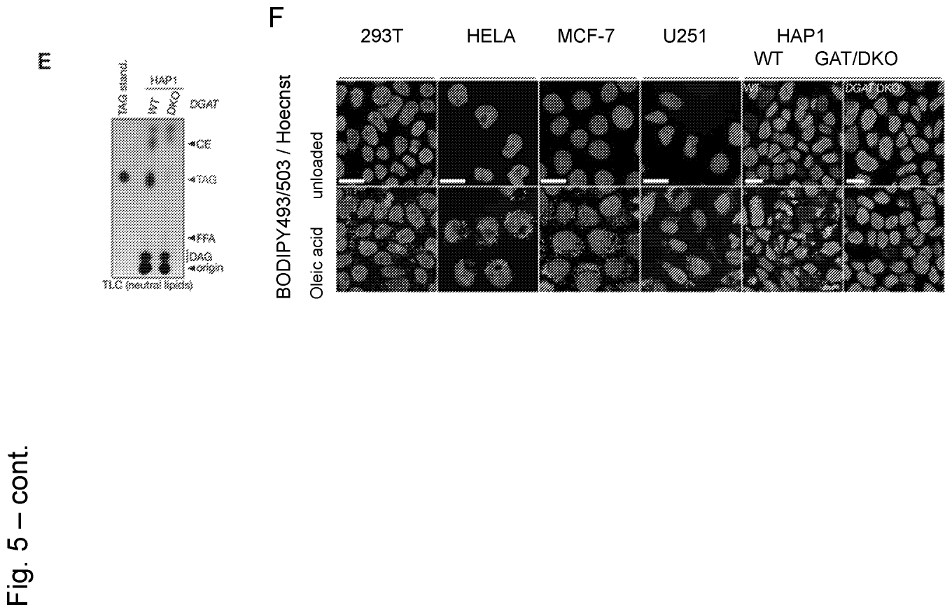

Acute inhibition of both DGATs (FIG. 5A) in human haploid (HAP1) cells severely reduced baseline TAG levels, as visualized by thin-layer chromatographic (TLC) separation of lipid extractions from these cells (FIG. 5B). We intriguingly observed a residual pool of TAG remaining in both HAP1 and 293T cells after multiple days of DGAT inhibition (FIG. 5C). In combination with the incomplete absence of TAGs in mice lacking both DGAT enzymes (16), this led us to investigate alternative mechanisms of TAG biosynthesis.

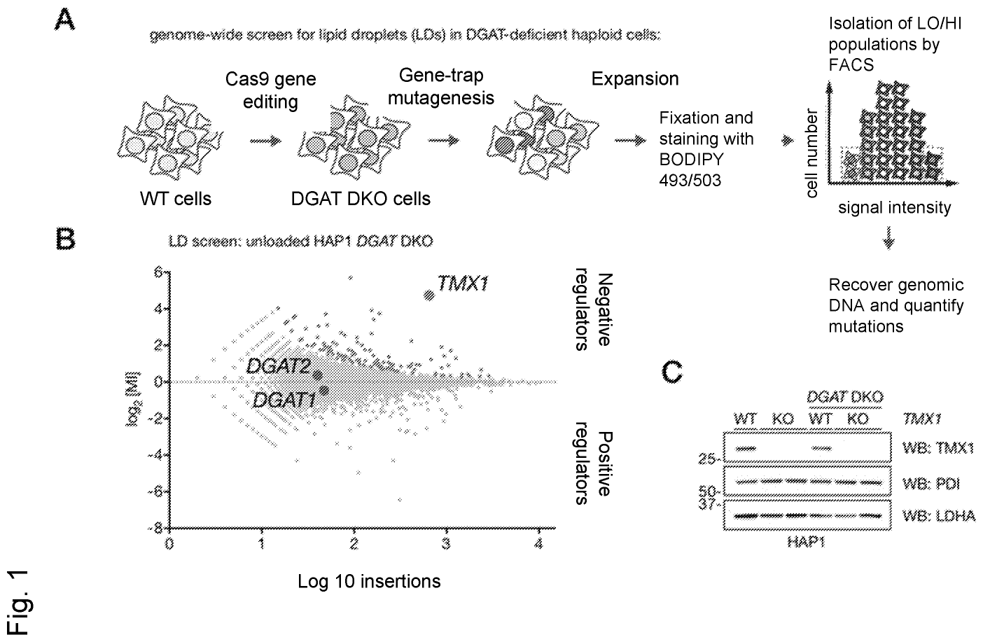

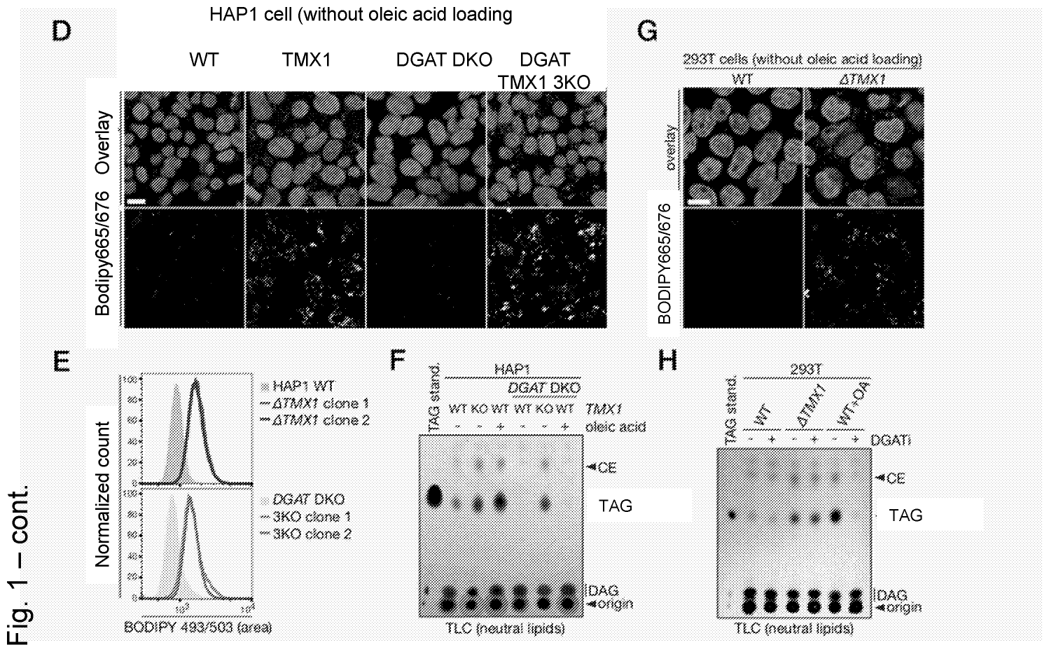

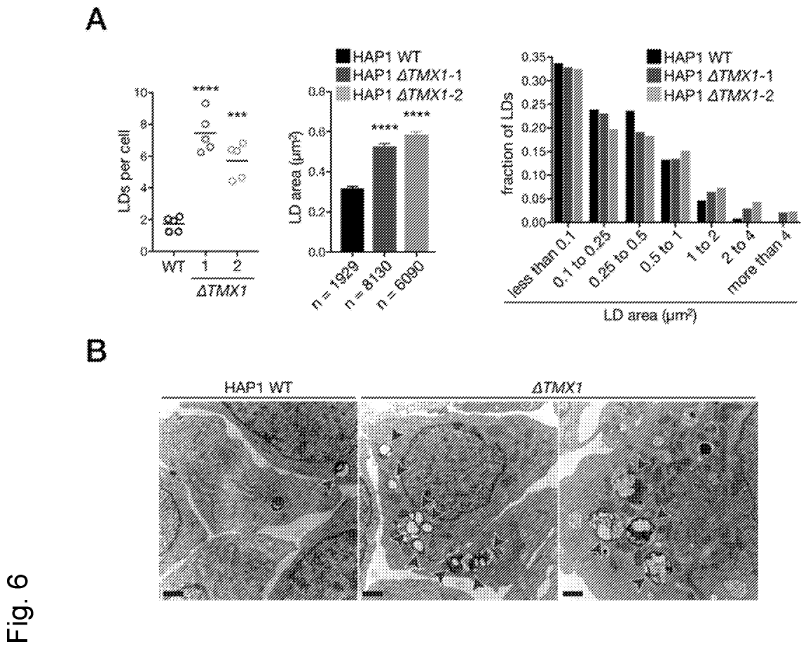

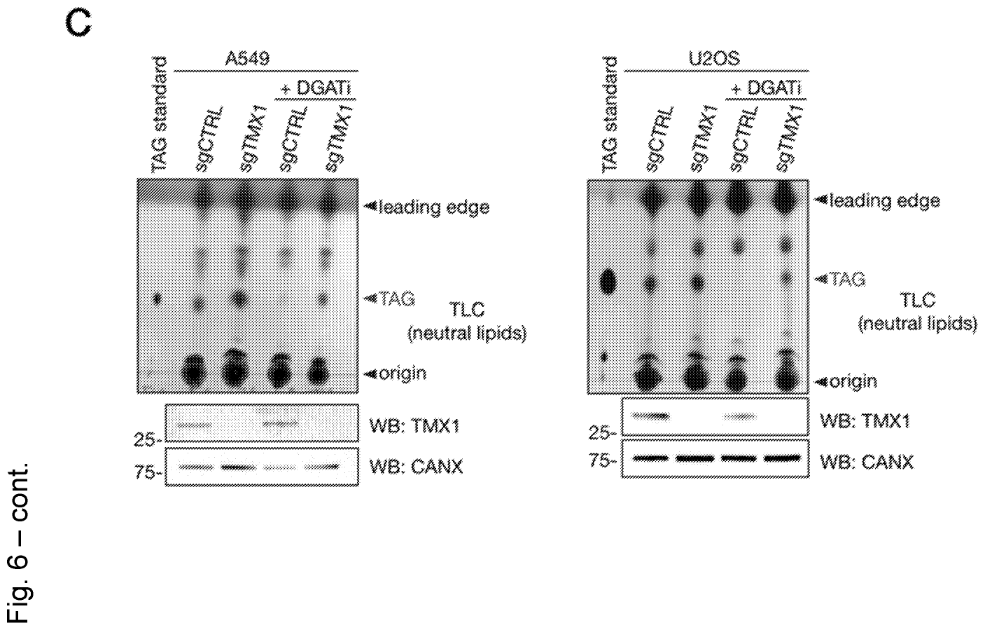

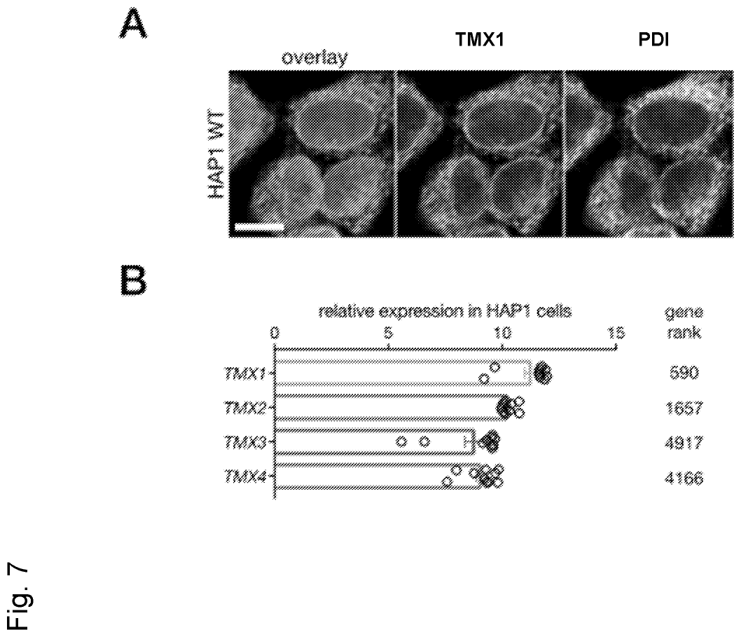

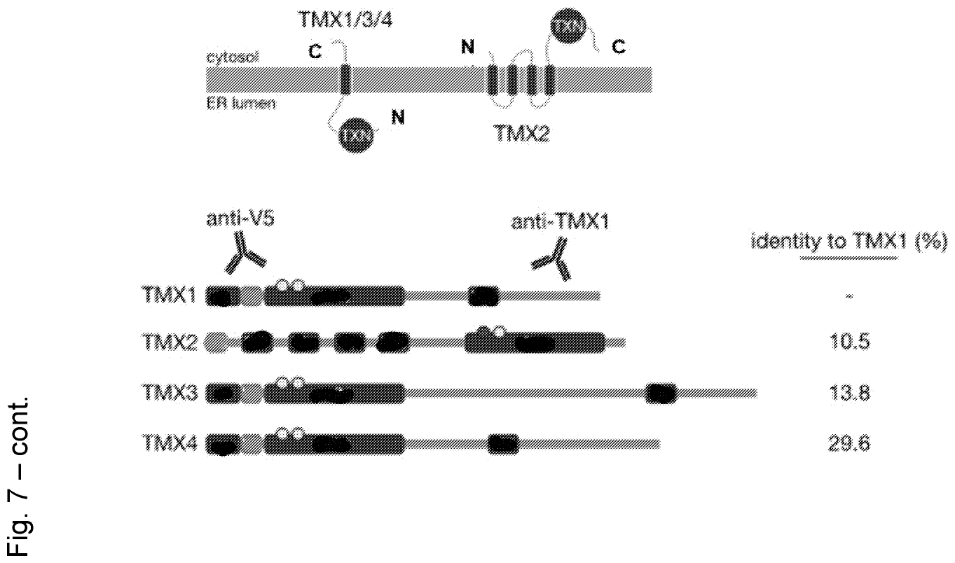

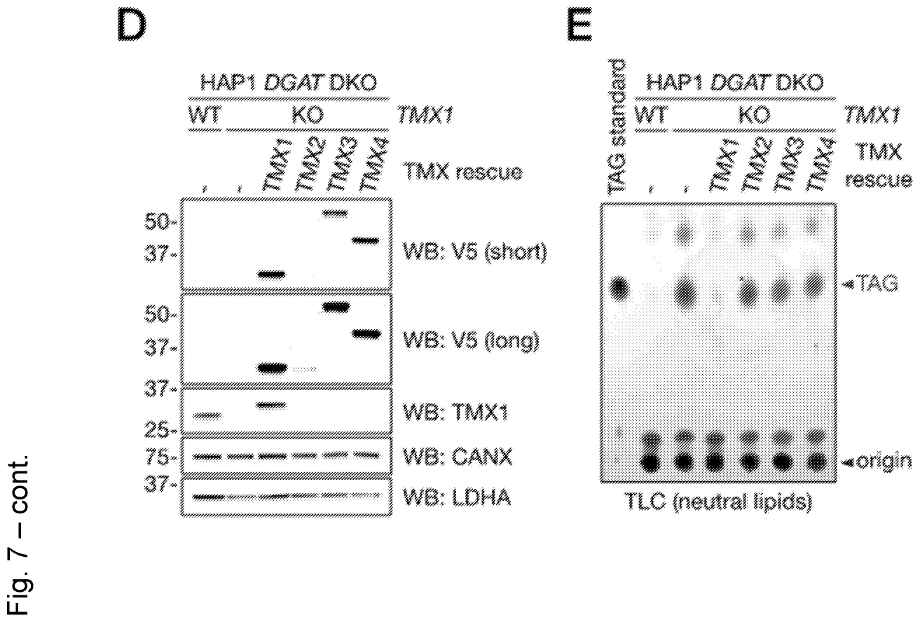

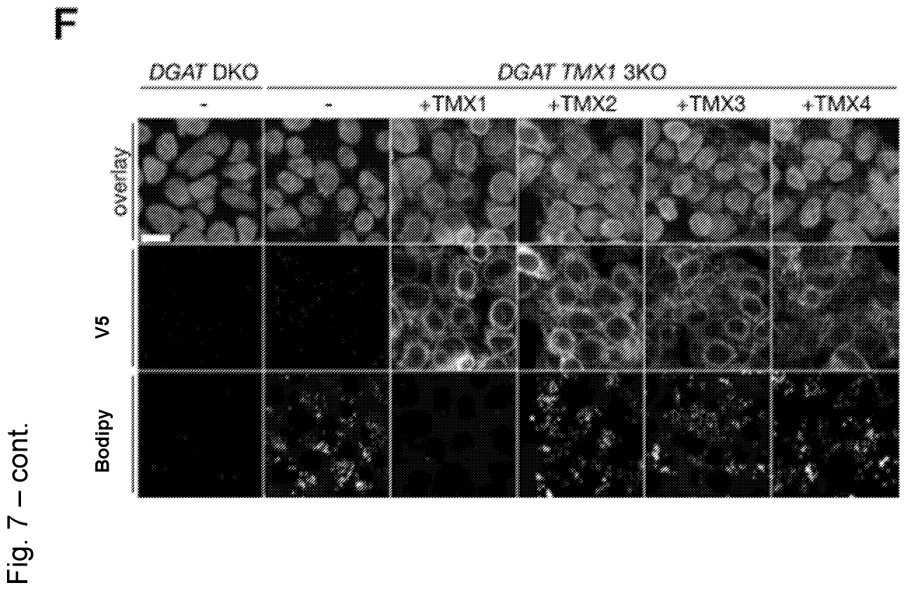

We designed a genetic screen in haploid human cells in order to identify regulators of alternative TAG accumulation, reasoning that carrying out this screen in cells devoid of DGAT1/2 enzymes would allow us to identify regulators specific to this non-canonical pathway. We thus deleted DGAT1 and DGAT2 (DGAT double knockout [DKO]; FIGS. 5D and E) in HAP1 cells, rendering them resistant to lipid droplet accumulation by the loading of free fatty acids (in the form of oleic acid; FIG. 5F) as this is expectedly a DGAT-dependent process (4). Applying gene-trap mutagenesis coupled to fluorescence-based cell sorting and deep sequencing (17), we used lipid droplets as a fluorescent surrogate for TAG levels (FIG. 1A). Strikingly, mutations in transmembrane thioredoxin 1 (TMX1) led to the accumulation of lipid droplets (i) independently of DGAT molecules and (ii) in the absence of free fatty acid loading (FIG. 1B), which was further validated by genetic disruption of TMX1 in either a WT or DGAT DKO background (FIG. 1C-E). Compared to their WT counterparts, TMX1 KO cells had on average 3- to 4-fold more lipid droplets that were larger in size; this however likely represented an underestimation as KO cells almost uniquely contained very large (>2 μm2) lipid droplets that, upon ultrastructural analysis, were found to be multiple, smaller structures clumped together (FIGS. 6A and B). TLC analysis from TMX1-null cells revealed a robust accumulation of TAG in the absence of DGATs, comparable to the amount of oleic acid-induced TAG in DGAT-competent cells (FIG. 1F). We additionally observed a similar induction in DGAT-independent TAG accumulation upon TMX1 disruption in 293T, A549 and U2OS cells (FIGS. 1G and H, and FIG. 6C). TMX1 encodes a transmembrane, ER-resident oxidoreductase (FIG. 7A), with its cysteine-containing thioredoxin (TXN) domain located within the lumen of the ER (18). While all TMX family members are expressed in HAP1 cells (17), TMX1 transcripts are the most abundant (FIG. 7B). Accordingly, we introduced other TMX family members into cells lacking TMX1 (FIG. 7C). Unlike TMX1, all other TMXs failed to suppress TAG and lipid droplet accumulation in these cells (FIG. 7D-F). Thus, TMX1 uniquely suppresses TAG accumulation in human cells via a mechanism that is independent of known TAG biosynthetic processes.

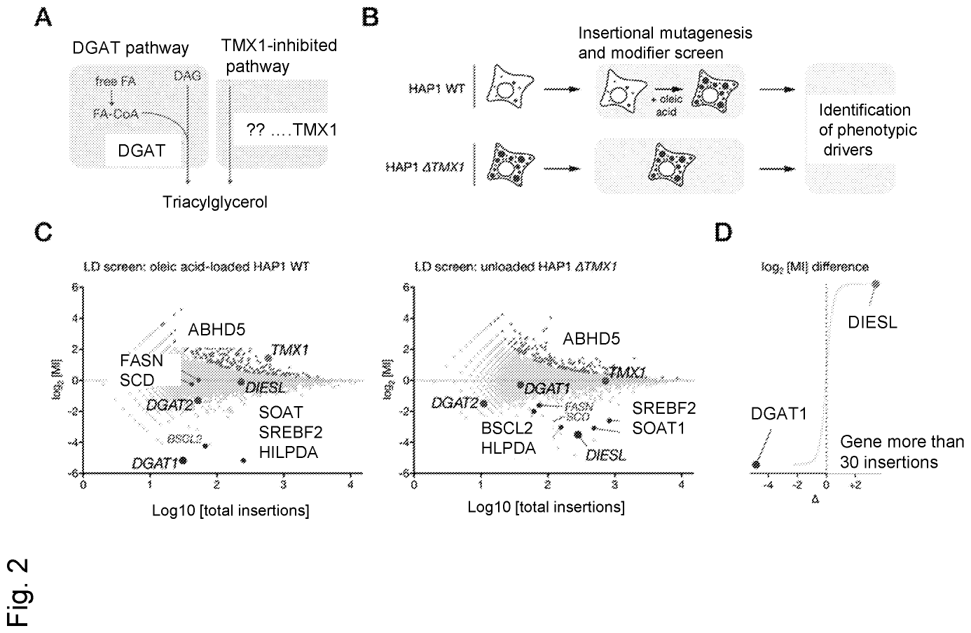

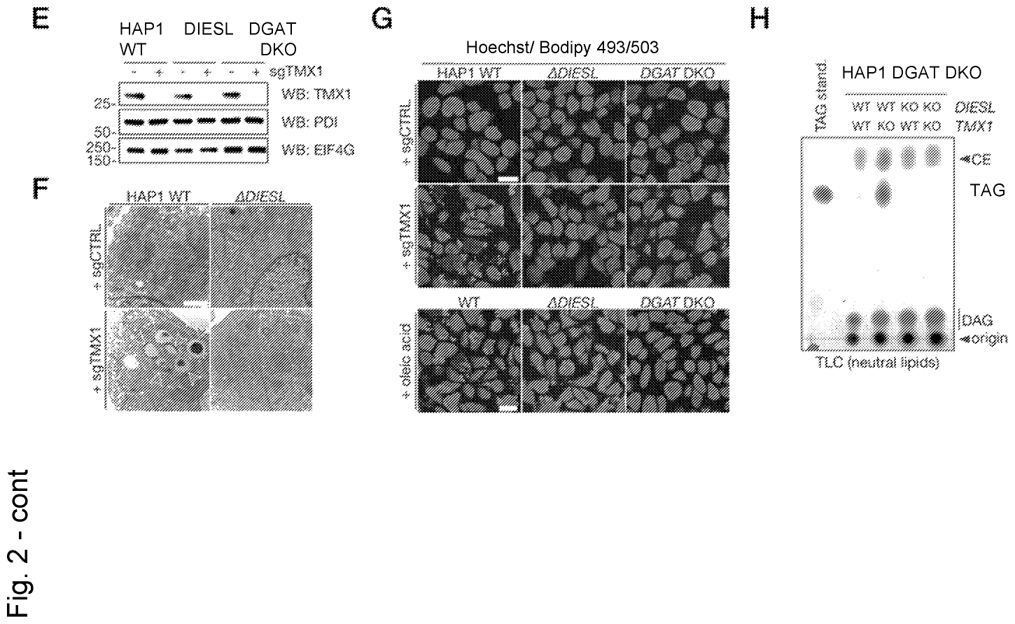

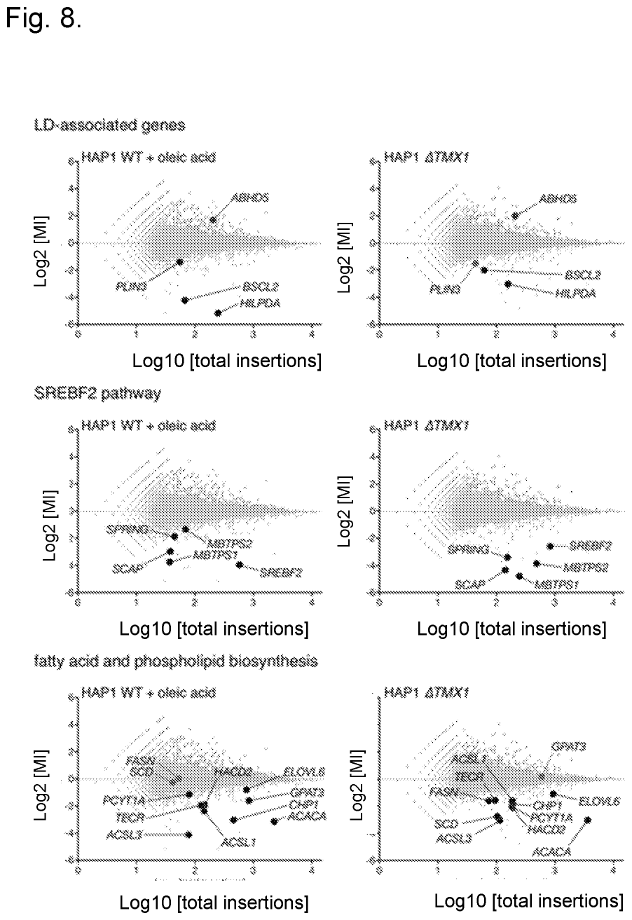

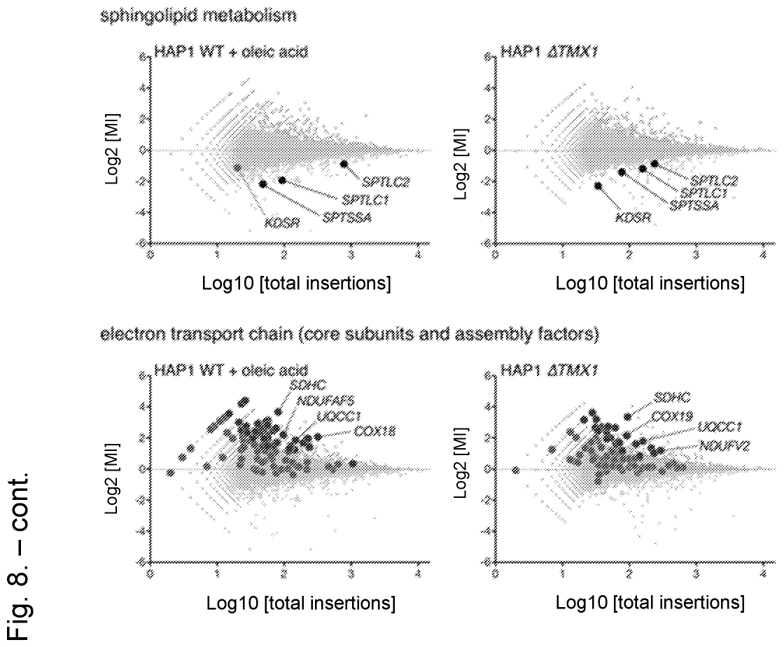

How do TMX1-deficient cells accumulate TAG (FIG. 2A)? To address this, we carried out a genetic suppressor screen to identify the underlying mechanism, again using lipid droplet accumulation as a readout, but now in TMX1-deficient cells. We then compared the results of this screen to the canonical DGAT pathway, using a screen in WT cells loaded with oleic acid (FIG. 2B). These two genetic maps (FIG. 2C) expectedly uncovered lipogenesis as a major driver of lipid droplet accumulation common to both backgrounds, as well as sphingolipid metabolism and mitochondrial electron transport as modest positive and negative regulators, respectively (FIG. 8). Comparison of the mutational biases of regulators in both screens identified DGAT1 as a top driver of lipid droplets in oleic acid-loaded cells (FIG. 2C) (4). Remarkably, our genetics revealed a functionally uncharacterized gene, TMEM68, as a selective driver of lipid droplet accumulation in the absence of TMX1 (FIG. 2C); we renamed this gene DGAT1/2-Independent Enzyme Synthesizing Storage Lipids (DIESL). DGAT1 and DIESL are the strongest selective drivers of lipid droplet accumulation in response to oleic acid loading and TMX1 loss, respectively (FIG. 2D). DIESL KO cells were resistant to lipid droplet accumulation upon TMX1 disruption (FIG. 2E-G) but were phenotypically-WT when loaded with oleic acid (FIG. 2G)—confirming the independence of the TMX1-DIESL pathway from the canonical DGAT pathway (7). In line with these observations, loss of TMX1 induces TAG accumulation that is carried out by DIESL rather than the DGAT enzymes (FIG. 2H).

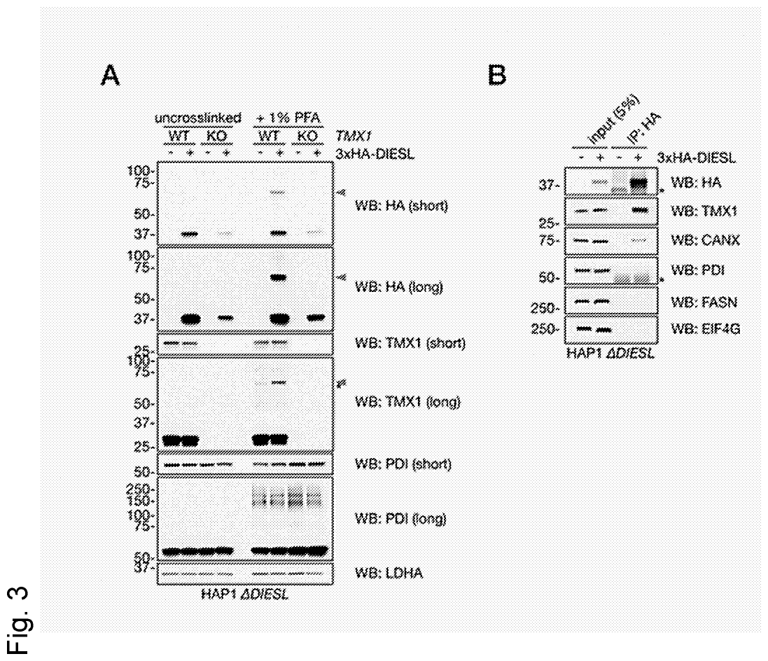

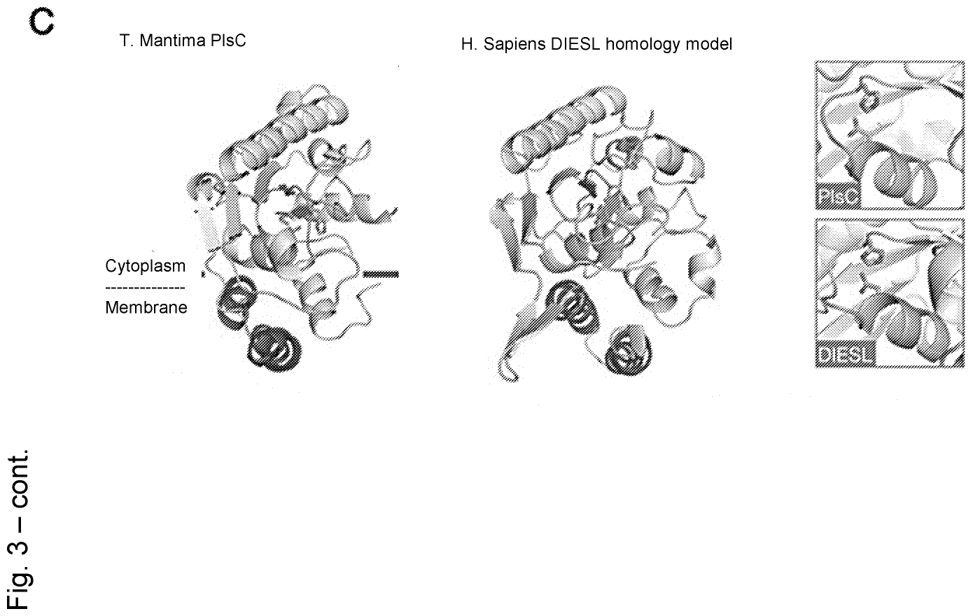

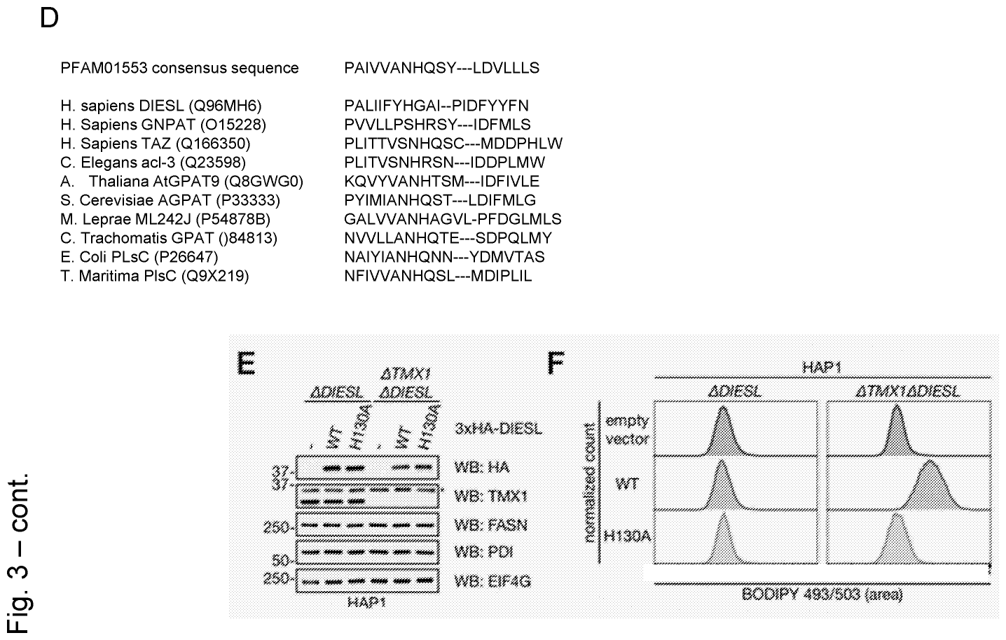

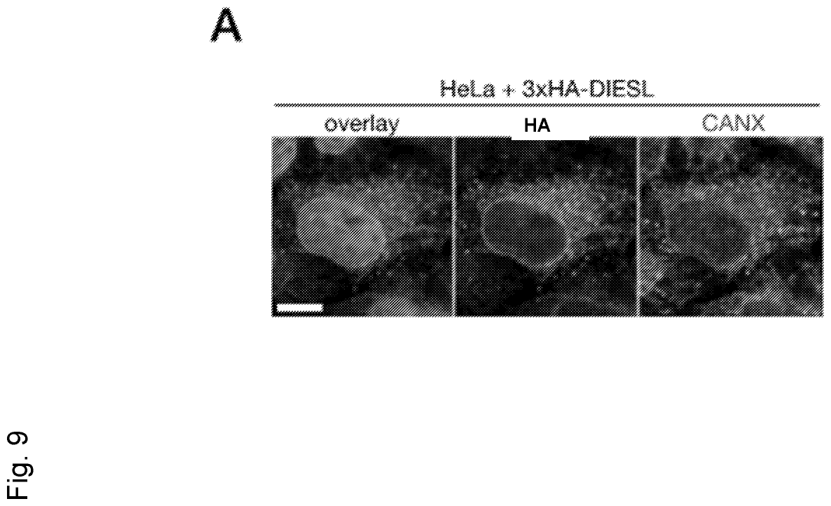

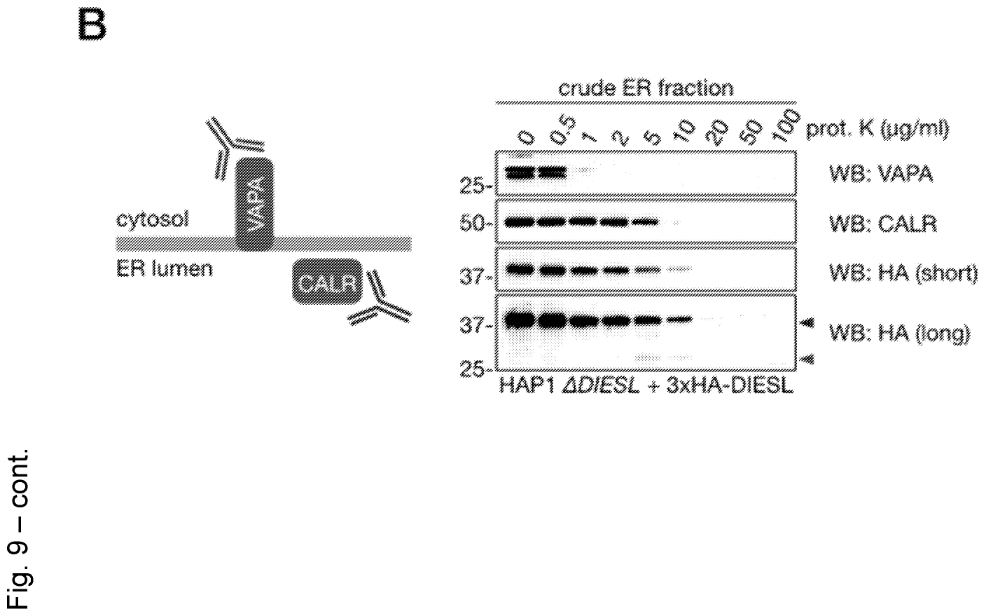

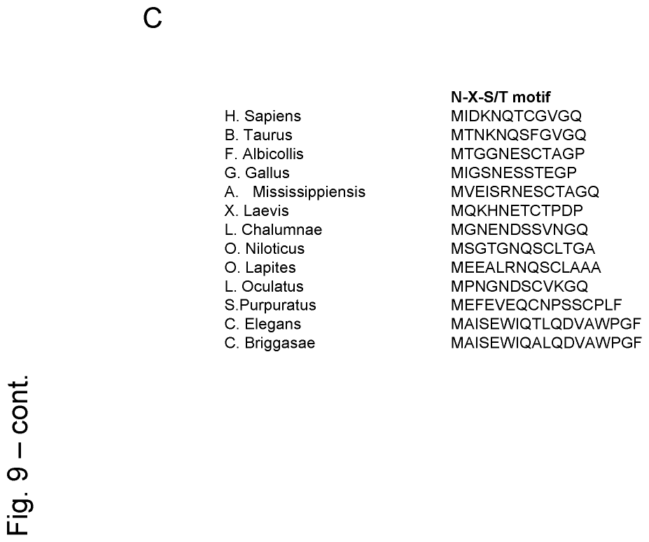

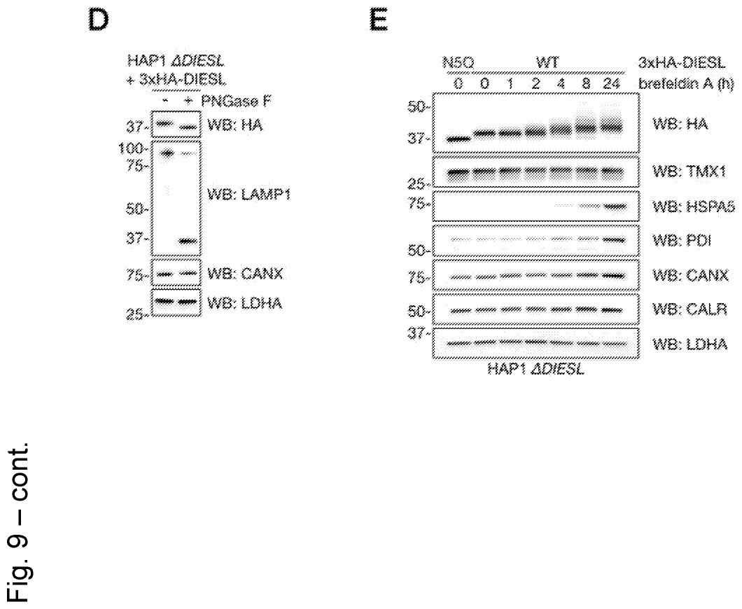

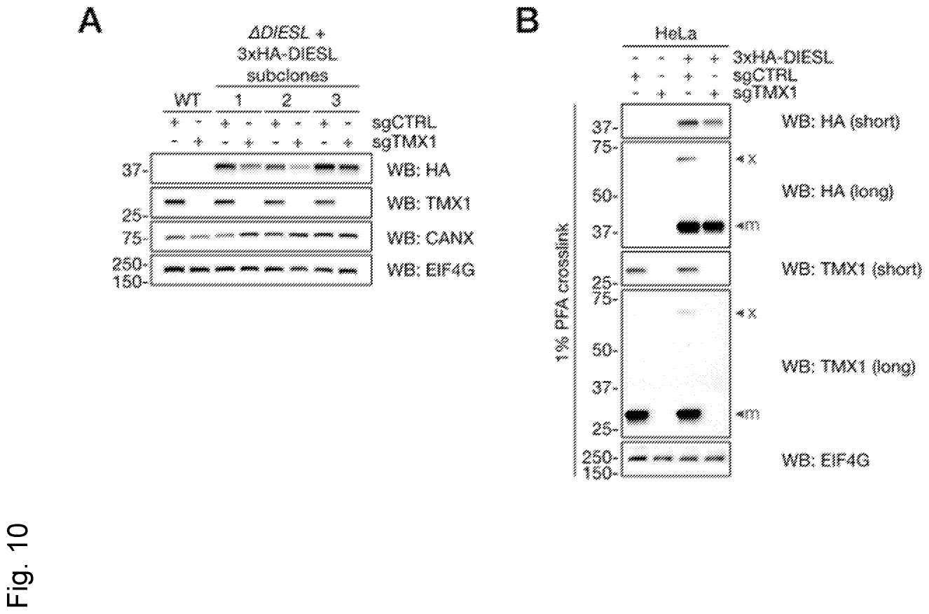

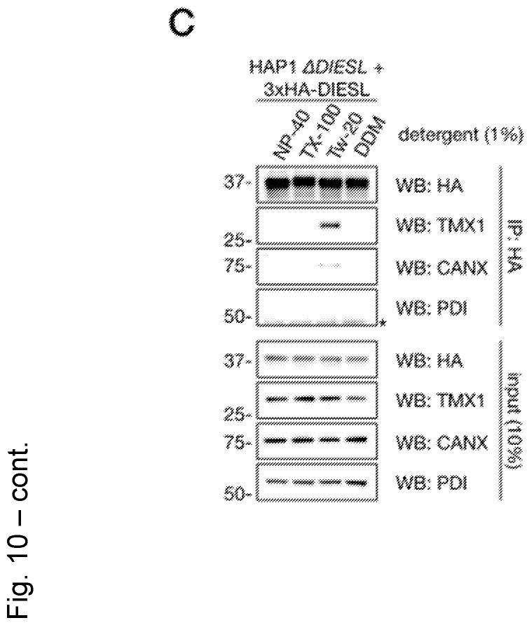

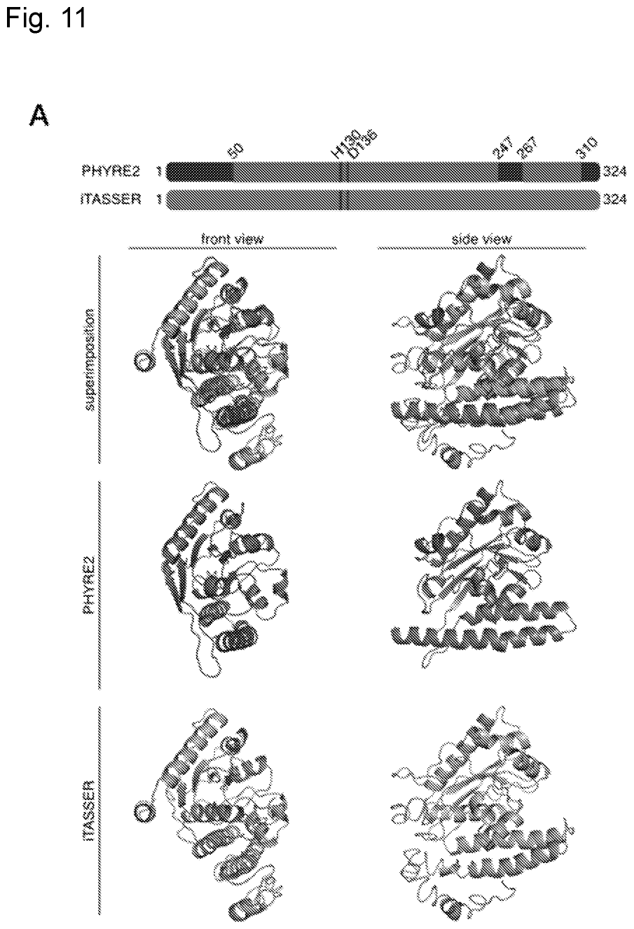

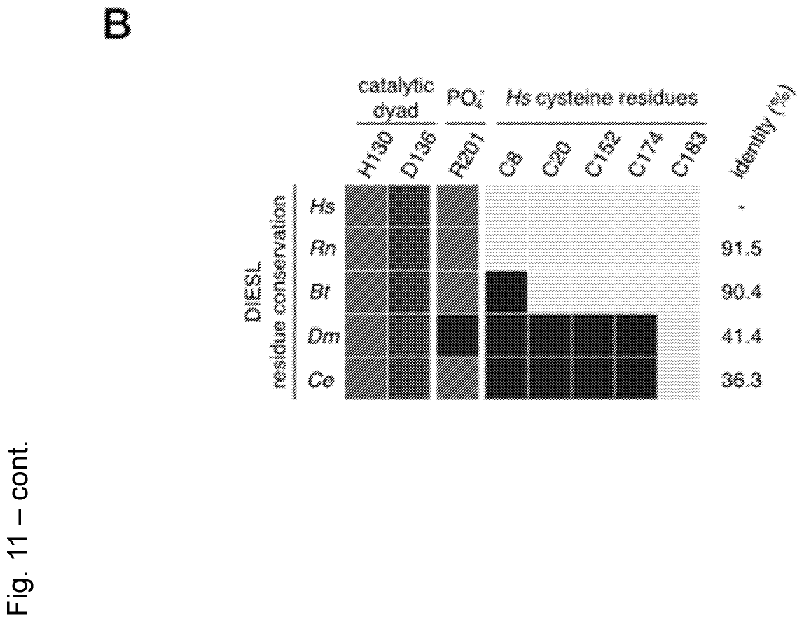

DIESL encodes a protein localized to the ER membrane (FIG. 9A) (19). A protease protection assay revealed that DIESL resides on the lumenal portion of the membrane in a purified microsomal preparation (FIG. 9B), which is further supported by N-glycosylation at its N-terminus (FIGS. 9C and D). We additionally observed brefeldin A-dependent elongation of DIESL-conjugated glycans (FIG. 9E), as would be expected if glycosylated DIESL faced the ER (rather than the Golgi) lumen (20). We observed that genetic disruption of TMX1 destabilized DIESL protein levels (FIG. 10A), suggesting a direct relationship. Analyzing DIESL binding partners by chemical crosslinking, we strikingly visualized a single, crosslinked band at ˜70 kDa by SDS-PAGE and subsequent immunoblotting (FIG. 3A). We determined that this band was a heterodimer composed of both TMX1 and DIESL as this ˜70 kDa band was detected by an antibody raised against TMX1, and additionally was absent from TMX1 KO cells (FIGS. 3A and 10B). Moreover, we were able to co-immunoprecipitate TMX1 with DIESL (FIG. 3B) in a detergent-dependent manner (FIG. 10C). Thus, both TMX1 and DIESL are proximal (accessible via formaldehyde crosslinking) and can interact in a membrane-dependent manner. DIESL has been characterized rudimentarily as an ER-resident acyltransferase of otherwise-unknown function (19). Homology modeling of DIESL demonstrated that the protein adopts an acyltransferase fold similar to a bacterial acylglycerol-phosphate acyltransferase (FIG. 11A)—although this family of enzymes catalyzes a diverse array of acylation reactions (21). From this model, we could identify the active site; a catalytic dyad composed of histidine-130 and aspartate-136 (FIGS. 3C and D, illustrated in FIG. 11B). Overexpressed DIESL only induced the accumulation of lipid droplets in the absence of TMX1, and this was abolished by mutation of the DIESL active site (H130A; FIGS. 3E and F). Thus, DIESL resides in an ER membrane complex with TMX1 and catalyzes DGAT-independent TAG accumulation in an enzymatic manner.

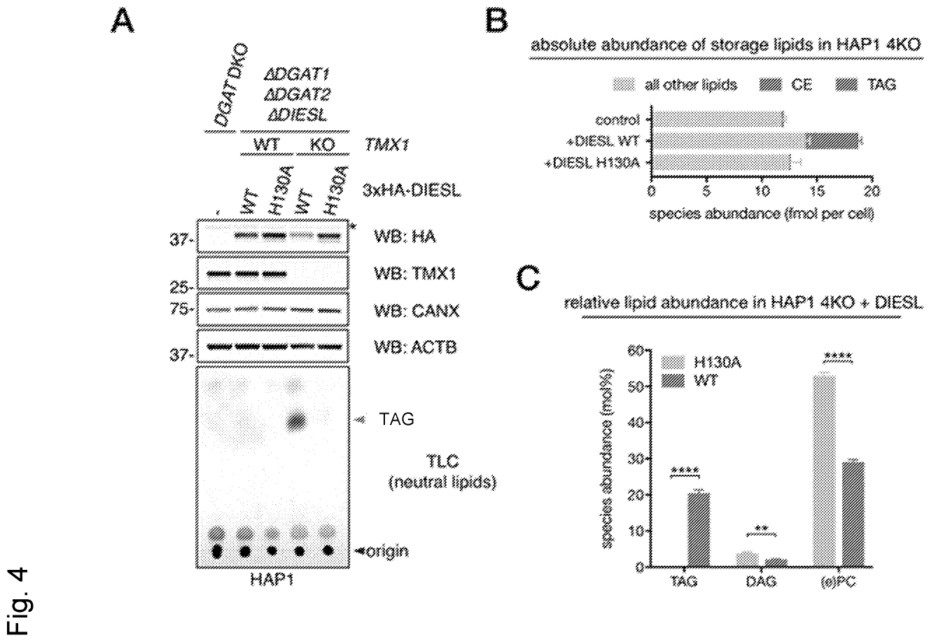

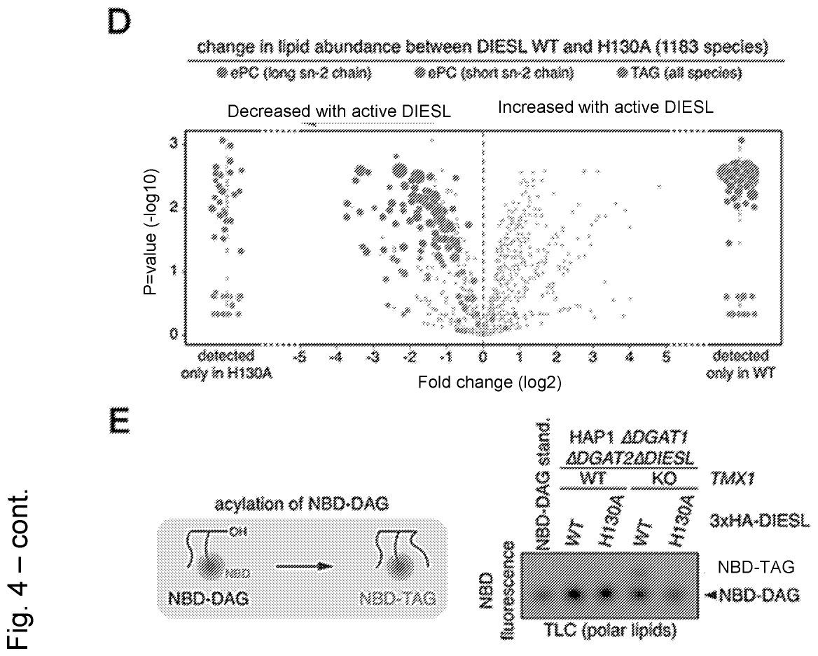

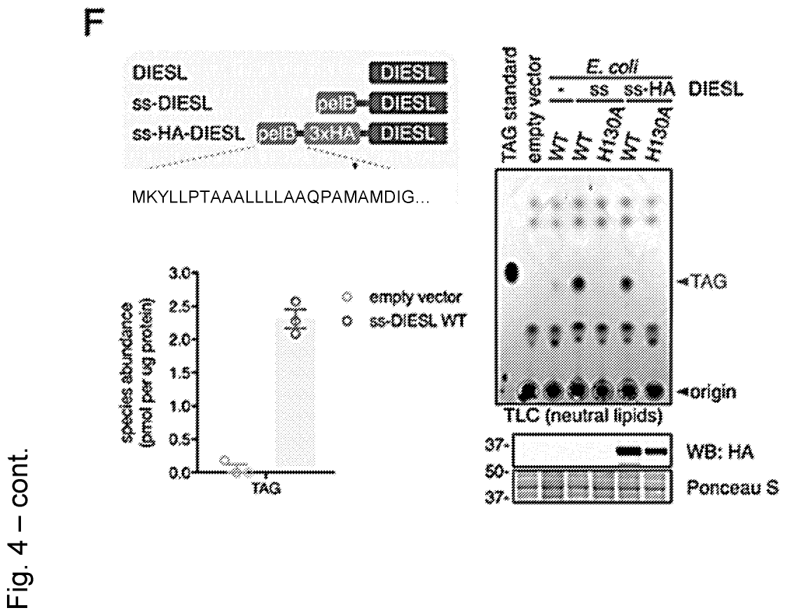

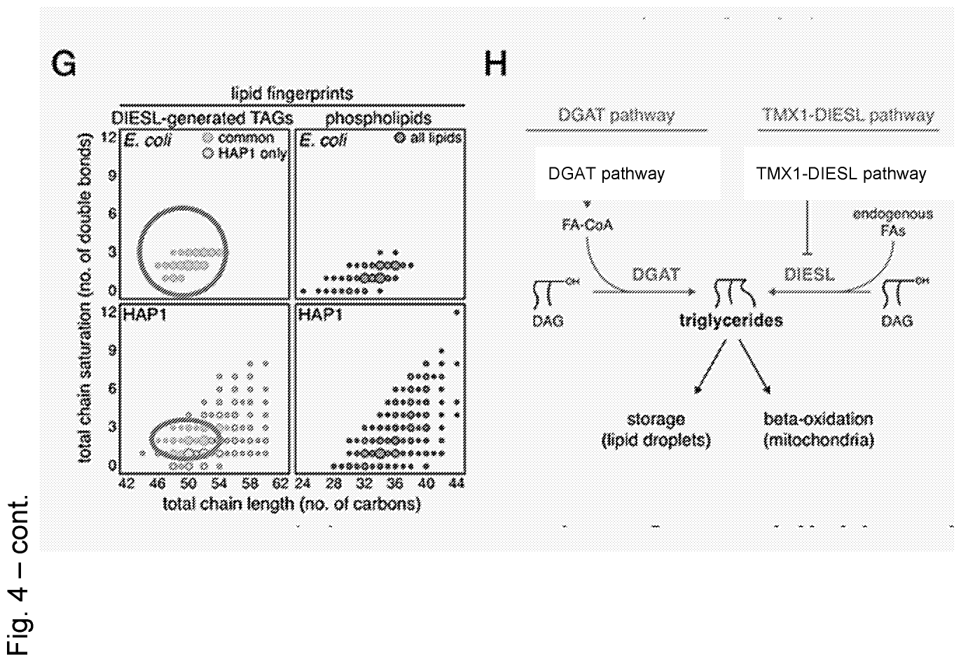

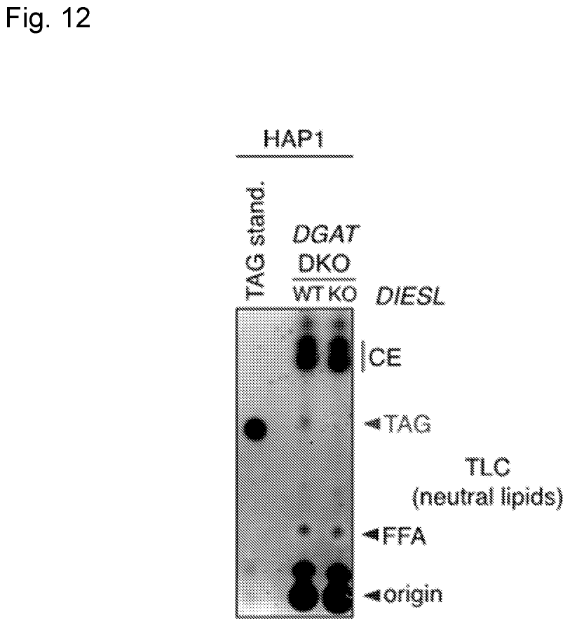

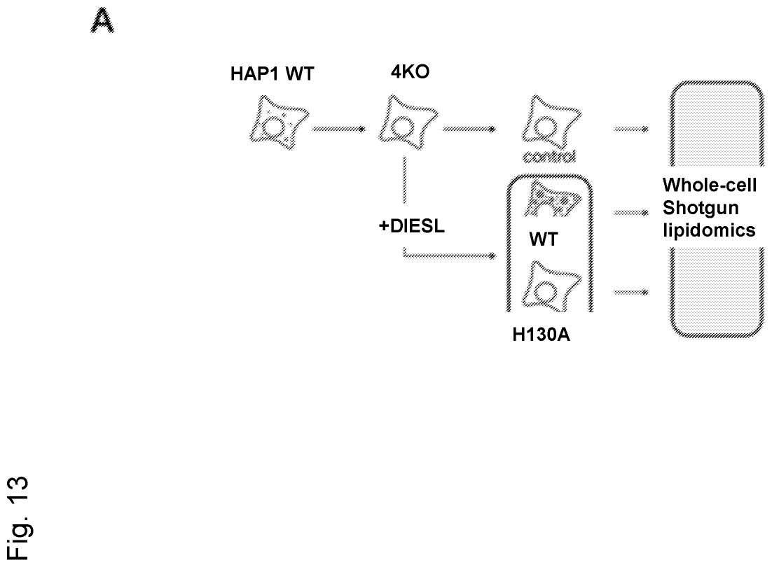

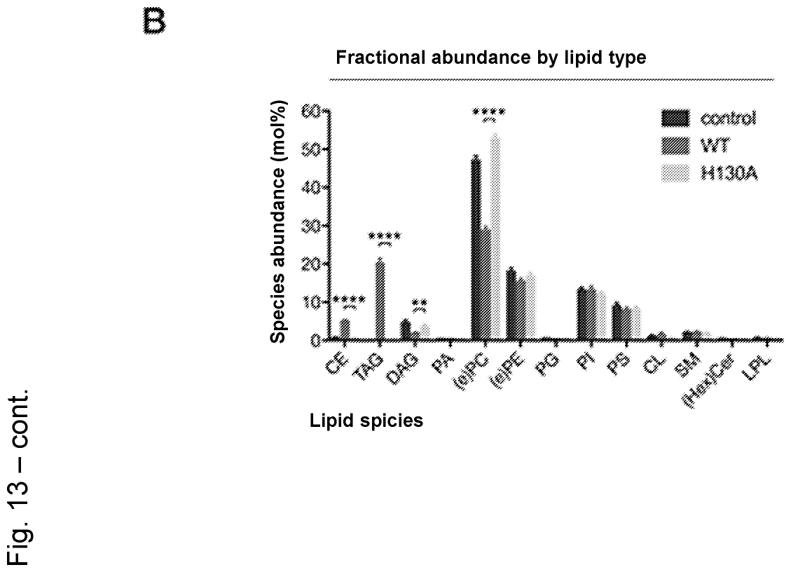

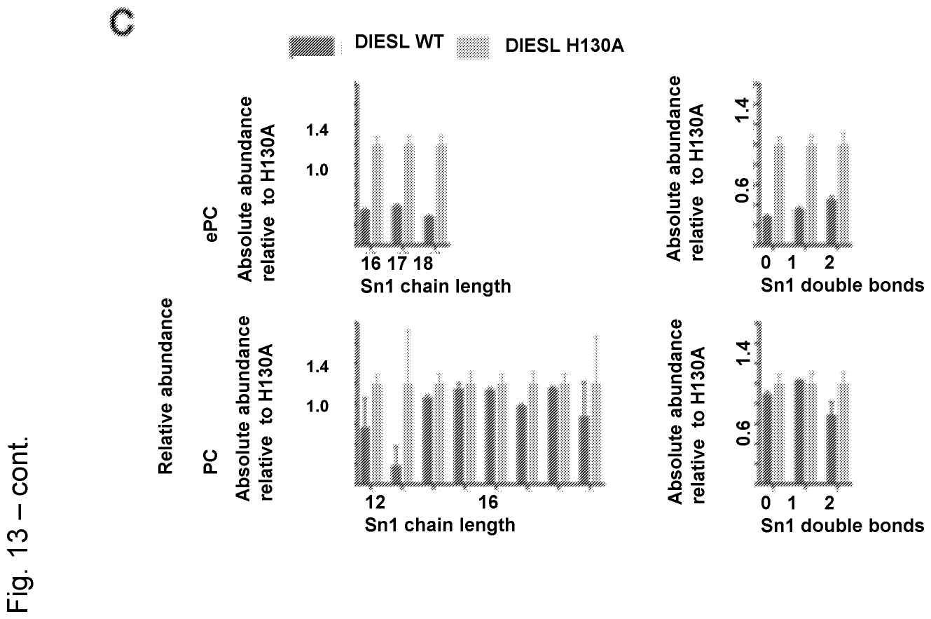

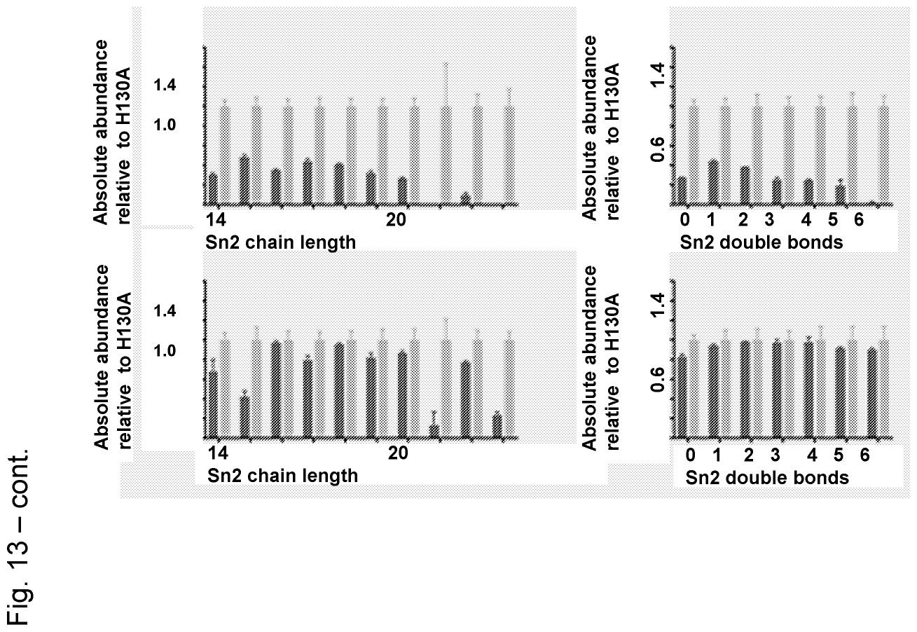

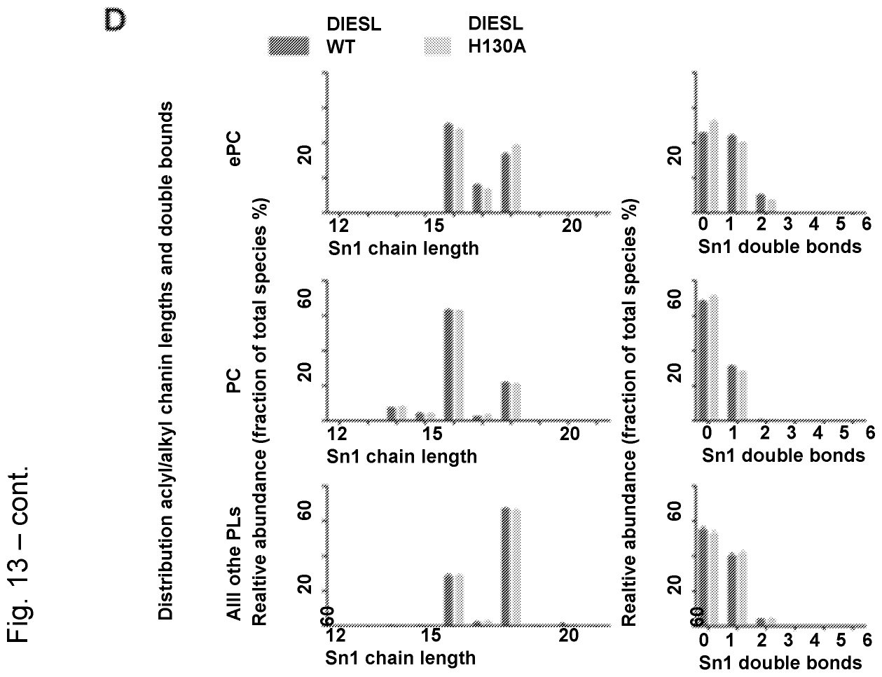

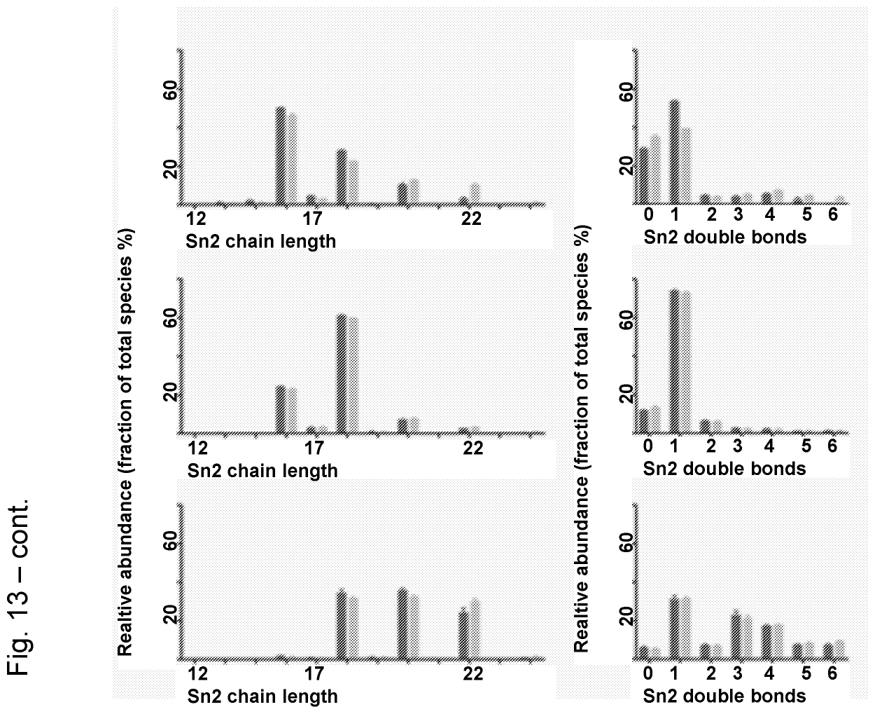

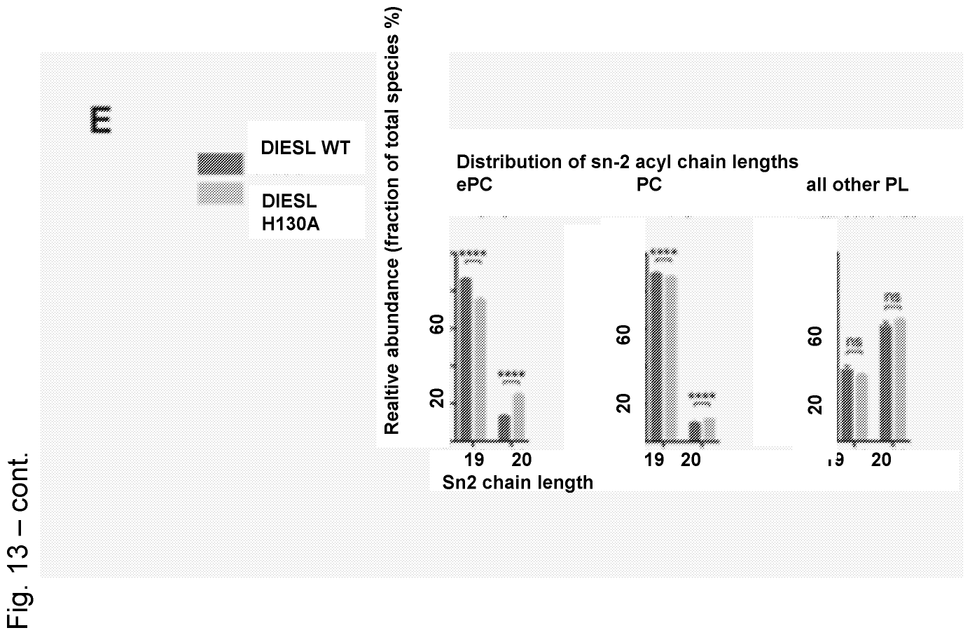

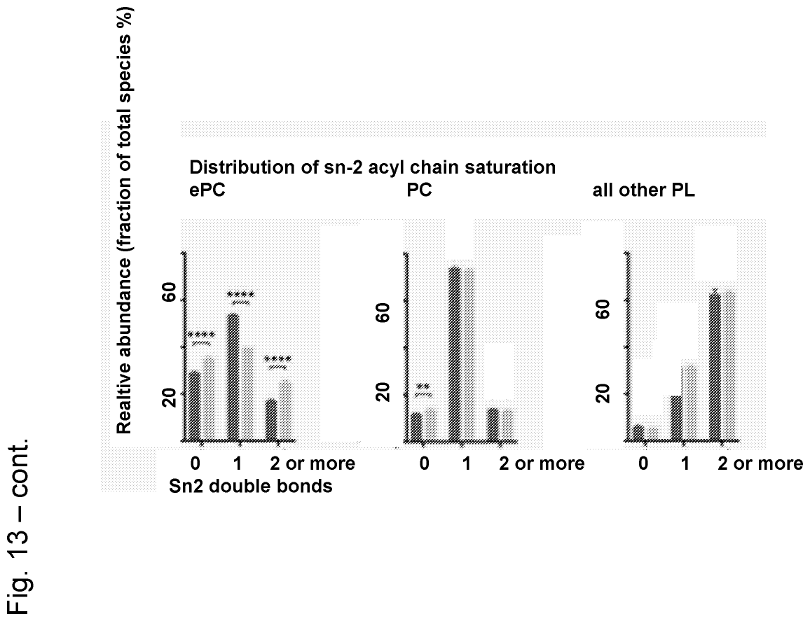

We next wanted to study the consequence of the activated DIESL acyltransferase on the cellular lipidome. We generated “TAG-null” HAP1 cells lacking these three enzymes (ΔDGAT1ΔDGAT2ΔDIESL; FIG. 12). Accordingly, we reintroduced either active or catalytic-inactive DIESL, in the presence or absence of TMX1, into these cells. Here, robust TAG accumulation occurred under the complete control of both the DIESL active site and its regulator, TMX1 (FIG. 4A). We then analyzed the lipidome in DIESL-reconstituted quadruple KOs (4KOs [ΔDGAT1ΔDGAT2ΔDIESLΔTMX1]), comparing the effect of reintroducing catalytic-active versus-inactive DIESL (outlined in FIG. 13A). The most pronounced change was observed on TAGs, which were undetectable in control (H130A) and became 20.5% of the lipidome in cells expressing active DIESL (FIG. 4B). This was accompanied by an increase in cholesteryl esters (FIG. 4B), a lipid species that co-accumulates with TAG in lipid droplets (8). To identify potential substrates of the DIESL, we looked at species that were specifically depleted by active DIESL. DIESL activity reduced the fractional abundance of only two lipid species; DAG and the predominant membrane phospholipid phosphatidylcholine (PC; FIG. 4C with complete lipidome in FIG. 13B-E), in particular, long-chain ether-linked PCs (FIG. 4D). Thus, lipidomic analysis demonstrates that DIESL primarily stimulates TAG abundance, potentially directly via the acylation of DAG using a phospholipid (or a phospholipid precursor) as an acyl donor.

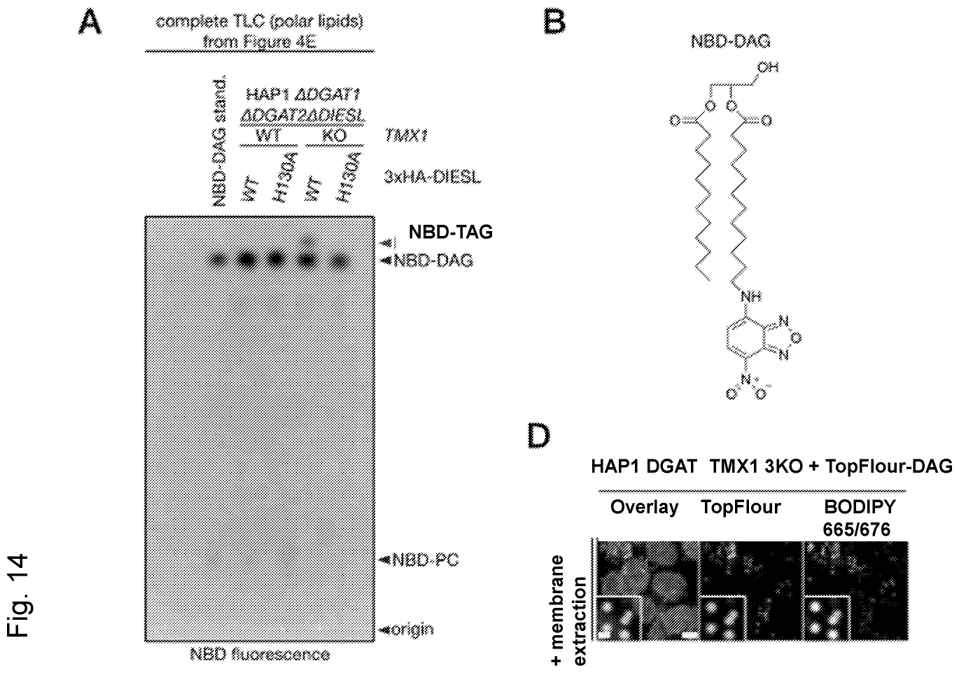

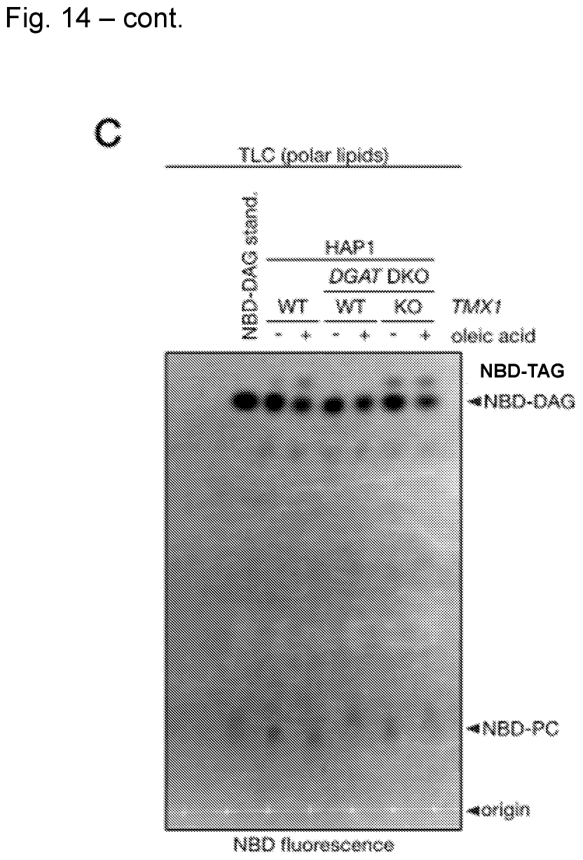

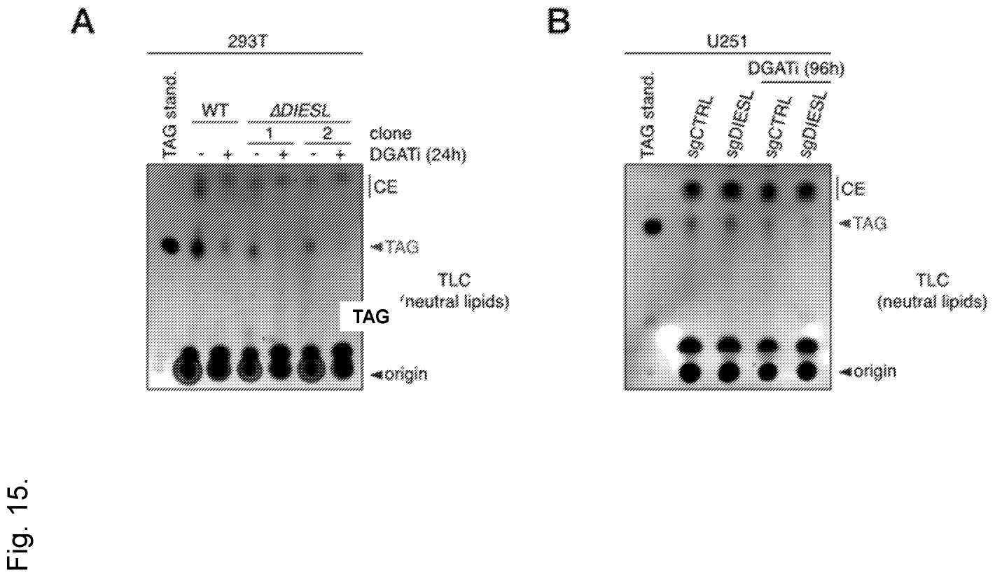

To characterize this enzymatic activity further, we examined the conversion of DAG to TAG. We reconstituted this acylation reaction in cells using fluorescent DAG (NBD-DAG), monitoring its conversion into TAG in TMX1-deficient cells in a manner dependent on DIESL enzyme activity (FIG. 4E and FIG. 14). Next, in order to establish DIESL as a bona fide TAG synthase, we reconstituted DIESL-dependent TAG synthesis in E. coli-a non-oleaginous organism lacking TAG synthesis genes (22). Expression of human DIESL in E. coli (preceded by a bacterial membrane targeting sequence) induced de novo TAG synthesis as determined by TLC and mass spectrometry (FIG. 4F). Given that E. coli is cultured in a lipid-free environment, this result demonstrated that the acyl donor is derived intrinsically. Indeed, in both human and bacterial cells, DIESL-synthesized TAGs reflected the cellular lipidome in length, saturation and complexity (FIG. 4G). We thus describe DIESL as a novel TAG synthase that uses endogenous lipids to acylate DAG (FIG. 4H).

Whereas DIESL is tightly controlled by its regulator TMX1 in the ER, it is not exclusively in the off-state in TMX1-expressing cells. While TAG synthesis is largely carried out by the DGATs in certain cell lines (HAP1 and U2OS), in others-such as 293T (FIGS. 15A) and U251 (FIG. 15B) cells-TAGs are synthesized by the combination of DIESL and DGATs at the steady-state. Moreover, DIESL-deficient mice display neurological deficits in their initial characterization (23). These data show that DIESL is already active under normal conditions, both in vivo and in certain cell lines that, in the case of 293T cells, can be further stimulated by relieving control by TMX1 (FIGS. 1G and H).

In summary, we observe alternative TAG synthesis in human cells, and find that it is carried out by a protein module within the ER that regulates the conversion of endogenous lipids into TAGs. In contrast to known TAG biosynthetic routes that are activated by high extracellular lipid supply, DIESL converts endogenous acyl chains into TAGs, altering the composition of cellular membranes, and should thus be tightly regulated. The potent negative regulator identified here-TMX1—may simultaneously clarify the fate of DIESL-produced TAGs, as TMX1 functions at ER-mitochondria membrane contact sites (24, 25). At these ER subdomains, TMX1 could coordinate the flow of lipids into mitochondria via DIESL-dependent TAG synthesis

The roles and regulation of DIESL will be important to study in detail. In animals, this pathway is connected to lipid-related traits. The obesity-associated mitochondrial NNT gene (26) shows genetic linkage with DIESL protein abundance in mice (27) and both DIESL and TMX1 loci have been associated with meat quality traits in cattle and fish (28-30). The profound implications of our understanding of the ER-regulated cholesterol biosynthetic pathway (31, 32) justify a detailed understanding of the herein-described TMX1-DIESL pathway of alternative triglyceride synthesis in humans.

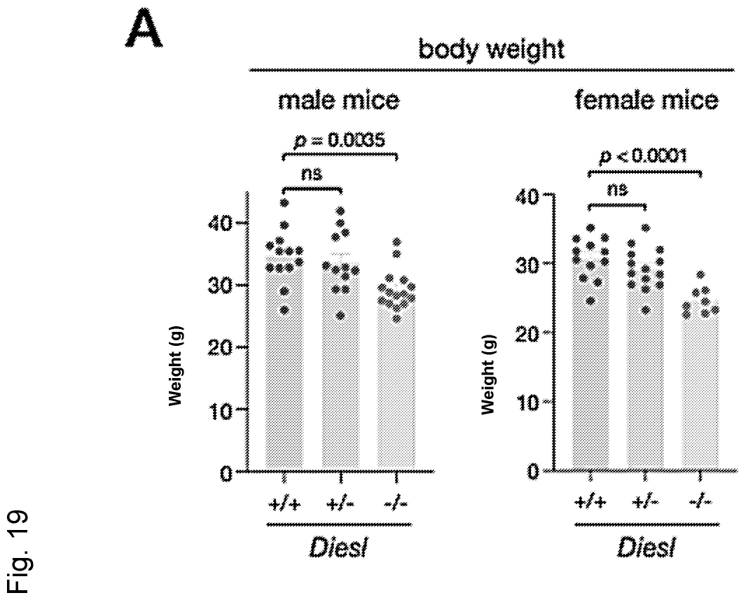

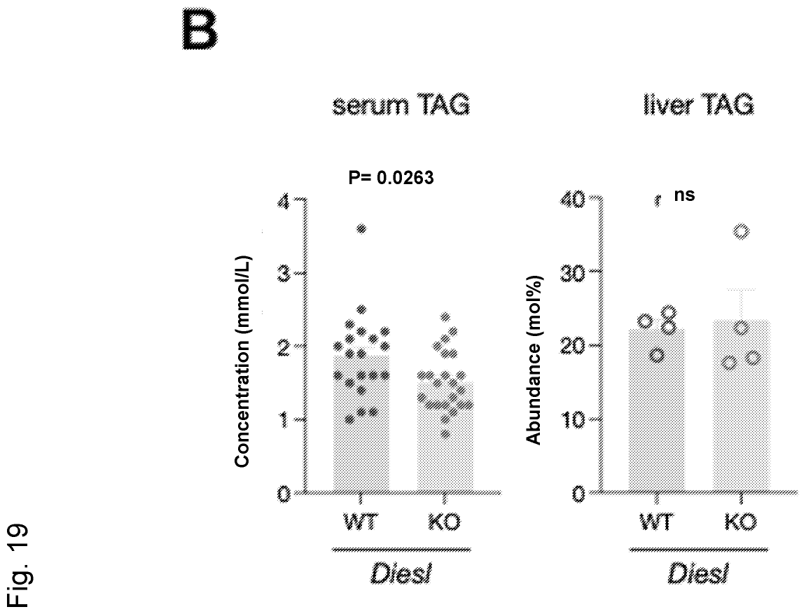

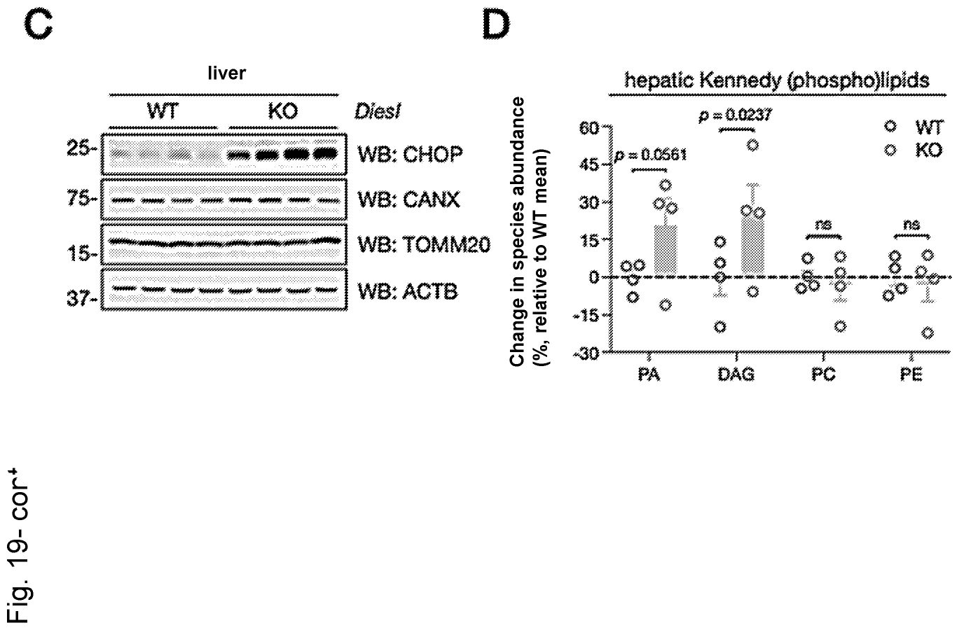

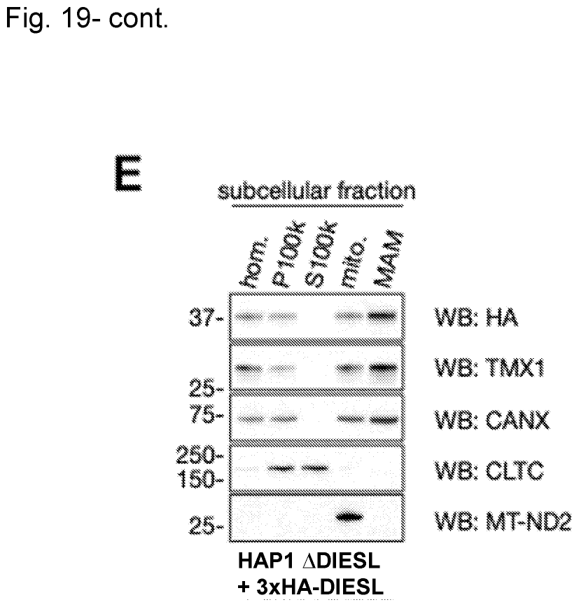

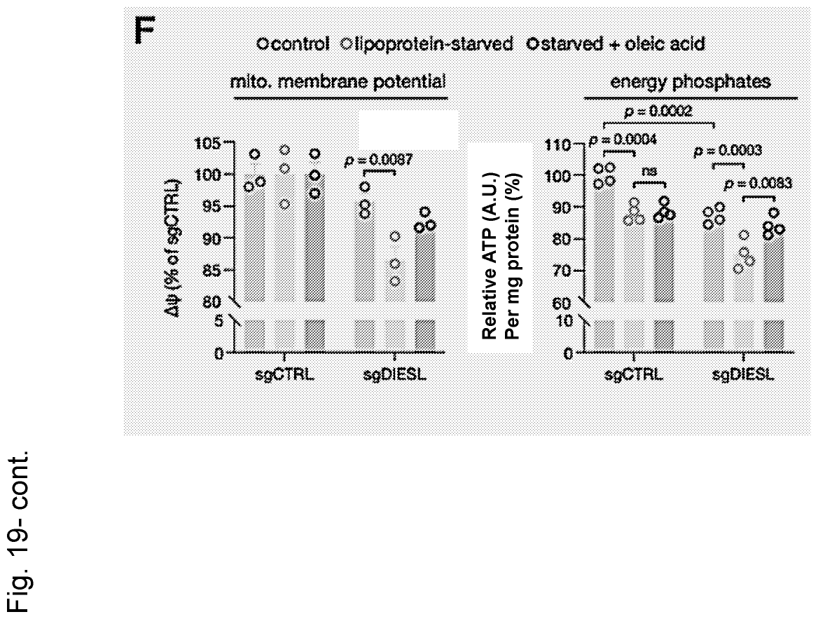

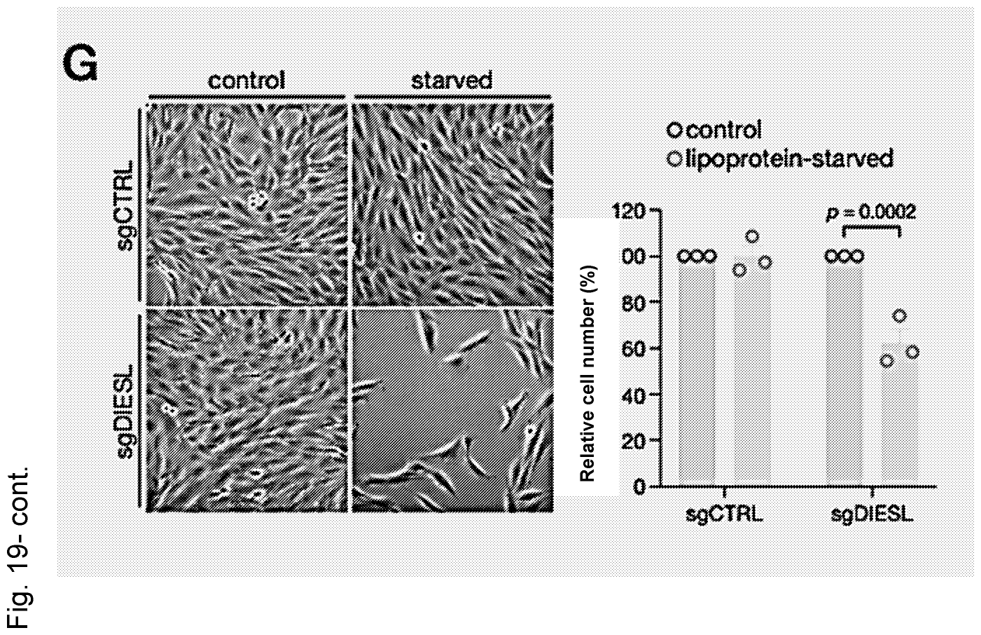

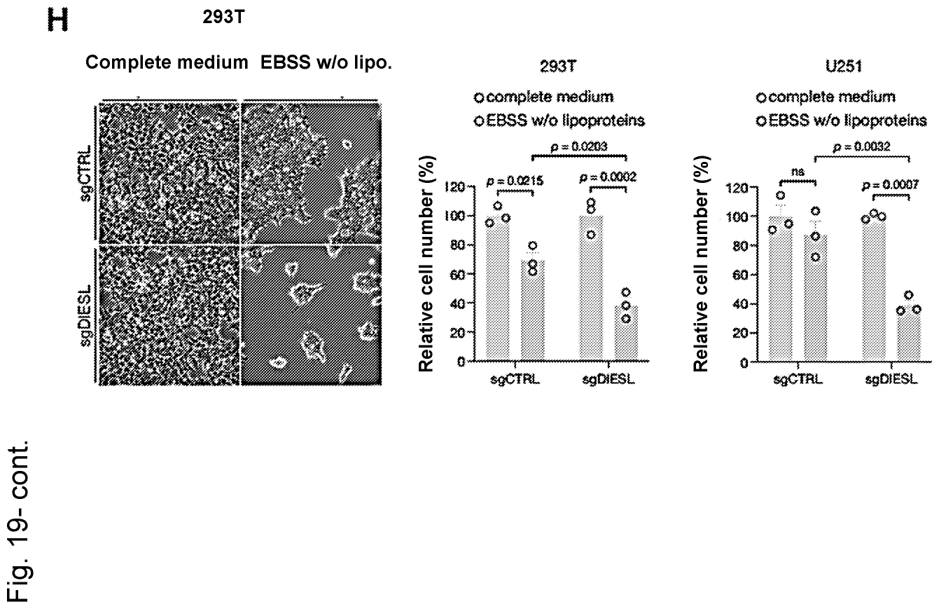

To examine a physiological role for DIESL, a knockout mouse was generated. Dies/KO mice were viable and born at the expected Mendelian ratio (p=0.8890 and 0.1638 for males and females, respectively; Chi-square test, n=68 mice in each condition). We initially observed a reduction in both male (17%) and female (19%) body weight in mice lacking DIESL (FIG. 19A). Having identified DIESL as a TAG synthase, we next focused on TAG homeostasis in these mice. We measured a selective decrease in TAG compared with cholesterol in serum lipid profiles (FIG. 19B), and whole-brain TAG was also reduced (data not shown). While the examined metabolic tissues (adipose tissue, liver, and skeletal muscle) of knockout mice were morphologically unaffected (data not shown), we detected very high levels of the stress-activated and metabolically-linked transcription factor CHOP (48) (49) in DIESL KO animals (FIG. 19C) suggesting that DIESL is normally active in this tissue. Whereas the stored levels of hepatic TAG were unaffected in the absence of DIESL (FIG. 19B), we measured a ˜25% increase in the levels of DAG—the DIESL substrate—in the liver (FIG. 19D). Thus, DIESL plays a role in normal physiology in mice by affecting body weight, hepatic DAG homeostasis and circulating triglycerides. Remarkably, we observed these phenotypes in mice where Tmx1 was expressed. Indeed, although DIESL appeared under complete inhibition by TMX1 in HAP1 cells, we were able to measure a marked contribution of DIESL to the cellular TAG pool in other cell lines (data not shown).

The effect of DIESL on TAG production may be underestimated by strictly examining the steady-state TAG pool, as triglycerides can be catabolized for energy. Interestingly, TMX1 has been implicated in the regulation of mitochondrial function (24), and both TMX1 (as previously shown) and DIESL reside at the mitochondria-associated membrane of the ER (FIG. 19E). We thus hypothesized that DIESL-generated TAGs may stimulate mitochondrial function, specifically when exogenous lipids are limiting, as DGAT activity is strongly boosted by external lipids. We first analyzed activation of AMP kinase, a master metabolic sensor that directly detects the energy state of the cell (50) (51), across various cell lines lacking DIESL, both cultured in lipoprotein-replete and -starved conditions. DIESL-deficient cells showed increased AMPK phosphorylation, either in RPE1 cells lacking DIESL grown under normal conditions, or in U251 and HT29 cancer cell lines starved of lipoproteins (data not shown). In 293T cells, AMPK activation was only observed in DIESL-deleted cells when lipoproteins were removed and the DGATs inhibited (data not shown). Focusing on untransformed RPE1 cells, we measured increased mitochondrial reactive oxygen species in the absence of DIESL; a feature that was exacerbated upon lipoprotein-starvation (data not shown). This starvation response did not yet cause widespread autophagy (data not shown). DIESL deficiency in RPE1 cells also decreased mitochondrial membrane potential and ATP levels when cells were cultured in the absence of lipoproteins (FIG. 19F). This was caused by depletion of fatty acids as these phenotypes could be rescued by the addition of oleic acid (FIG. 19F). The oleic acid rescue was sensitive to inhibition of CPT1A by etomoxir, demonstrating that this was functioning via fatty acid uptake by mitochondria (data not shown). RPE1 cells lacking DIESL cultured under lipoprotein-depleted conditions affected cell number (FIG. 19G). Culturing more resilient cell lines, such as 293T and U251 cells, under more severe starvation conditions (glucose reduction in the absence of amino acids and lipoproteins) also sensitized them to DIESL loss (FIG. 19H). Thus, during various starvation stimuli across different cell types, DIESL contributes to mitochondrial function, energy levels and cell fitness.

Loss of DIESL leads to deleterious phenotypes in mice, indicating that it is already active during normal physiology when TMX1 is present. Accordingly, it is likely that DIESL activity is regulated when needed.

TAG production counteracts lipotoxicity and ER stress (52). DIESL may detoxify the ER membrane, overseeing the removal of DAG and/or toxic acyl chains in a manner potentially sensed by the TMX1 redox cycle, and defects in this process may activate an ER stress response. In the livers of DIESL KO mice, we observed both elevated levels of the DIESL substrate DAG and the transcription factor CHOP. Furthermore, TAG catabolismcontributes to energy production, and across several cell systems we observed lessened mitochondrial function in the absence of DIESL, especially when starvation was applied. It is thus of interest to examine DIESL deficiency in a physiological context upon challenges, either in the form of nutrient deprivation (or imbalance), or ER stress conditions.

### Materials and Methods

### Cell Culture, Reagents and Antibodies

HAP1 cells were maintained in IMDM supplemented with 10% FBS, L-glutamine, penicillin and streptomycin, and cultured at 37° C. in 5% CO2. 293T, A549, HeLa, U251 and U2OS cells were maintained in DMEM under the same conditions. Cell lines used in this study were routinely monitored for mycoplasma contamination. BODIPY 493/503 and 665/676 neutral lipid dyes, such as TMRM and MitoTracker Red CM-H2XROS, were purchased from ThermoFisher. Brefeldin A, CCCP, etomoxir, lipoprotein-deficient fetal calf serum, oleic acid, PF-06424439 (DGAT2 inhibitor) and phosphatase inhibitor cocktail were purchased from Sigma-Aldrich. A-922500 (DGAT1 inhibitor) was purchased from Selleck. Protease inhibitor cocktail was purchased from Roche. Triacylglycerol (triolein) and diacylglycerol (diolein) were obtained from Avanti Polar Lipids. [14C] DAG (1,2,-dioleoyl-rac-glycerol) was purchased from American Radiolabeled Chemicals. Antibodies used in this study were anti-ACTB (Abcam, ab6276), anti-CALR (BD Transduction Laboratories, 612136), anti-CANX (Abcam, ab22595), anti-alpha-tubulin (Santa Cruz Biotechnology, sc-32293), anti-AMPK (Cell Signaling Technology, 2532), anti-AMPK pT172 (Cell Signaling Technology, 2535), anti-CLTC (Thermo Fisher, PA5-17347), anti-EIF4G (Cell Signaling Technology, 2498), anti-FASN (Santa Cruz Biotechnology, sc-55580), anti-HA (Biolegend, 901503), anti-HSPA5 (Cell Signaling Technology, 3177), anti-LAMP1 (Santa Cruz Biotechnology, sc-19992), anti-LDHA (Cell Signaling Technology, 3582), anti-PDI (Abcam, ab2792), anti-TMX1 (Atlas Antibodies, HPA003085 and Origene, TA507042), anti-TOMM20 (Abcam, ab186735), anti-V5 (ThermoFisher, 14-6796-82) and anti-VAPA (Atlas Antibodies, HPA009174).

### Plasmids and Cloning

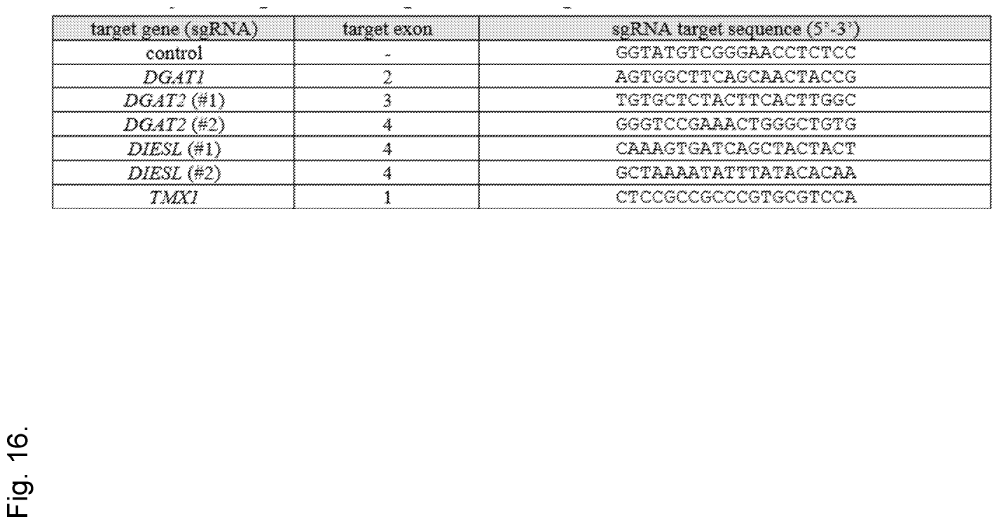

Synthetic guide RNAs (sgRNAs) were purchased as short ssDNA with sticky ends, annealed and cloned into pX330 and pLentiCRISPRv2 (with puromycin, blasticidin or mCherry selection markers) cut with Bbsl or BsmBI (New England BioLabs), respectively. As a non-targeting control sgRNA (sgCTRL), a sequence targeting the zebrafish TIA gene was used (33). A complete list of sgRNAs used in this study, generated using the Broad Institute's Genetic Perturbation Platform (portals.broadinstitute.org/gpp/public), are listed in FIG. 16. 3xHA-tagged DIESL (Q96MH6-1) and V5-tagged TMX1 (Q9H3N1), TMX2 (Q9Y320-1), TMX3 (Q96JJ7-1), and TMX4 (Q9H1E5) were purchased as linear DNA flanked by Nhel and Agel restriction sites from Integrated DNA Technologies, digested and cloned into pLEX305 backbones. The N5Q and H130A DIESL mutants were generated by site-directed mutagenesis using the QuikChange II kit (Agilent). For recombinant expression of DIESL, human DIESL was codon-optimized for E. coli expression and pET24 constructs encoding DIESL, pelB-DIESL and pelB-3xHA-DIESL were purchased as plasmids from Twist Bioscience.

### Generation of Clonal Knockout Cell Lines

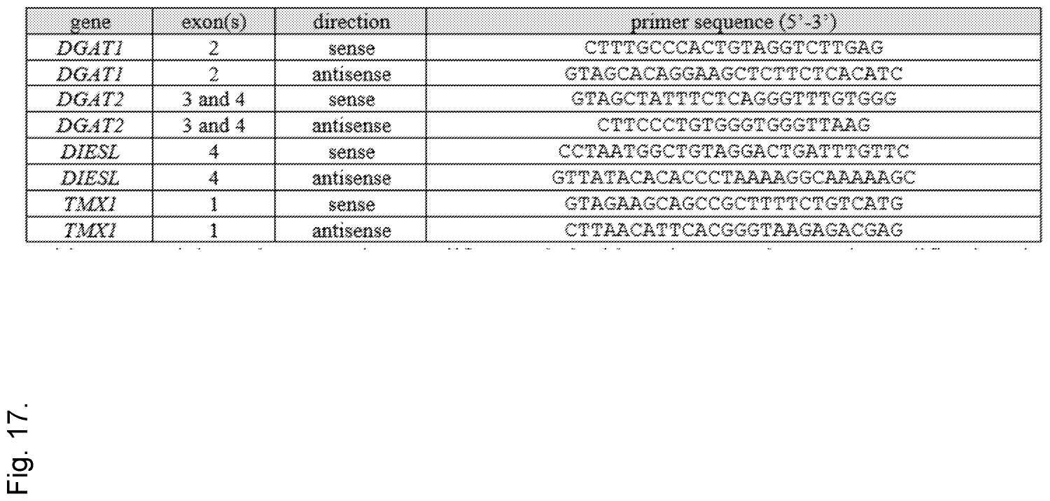

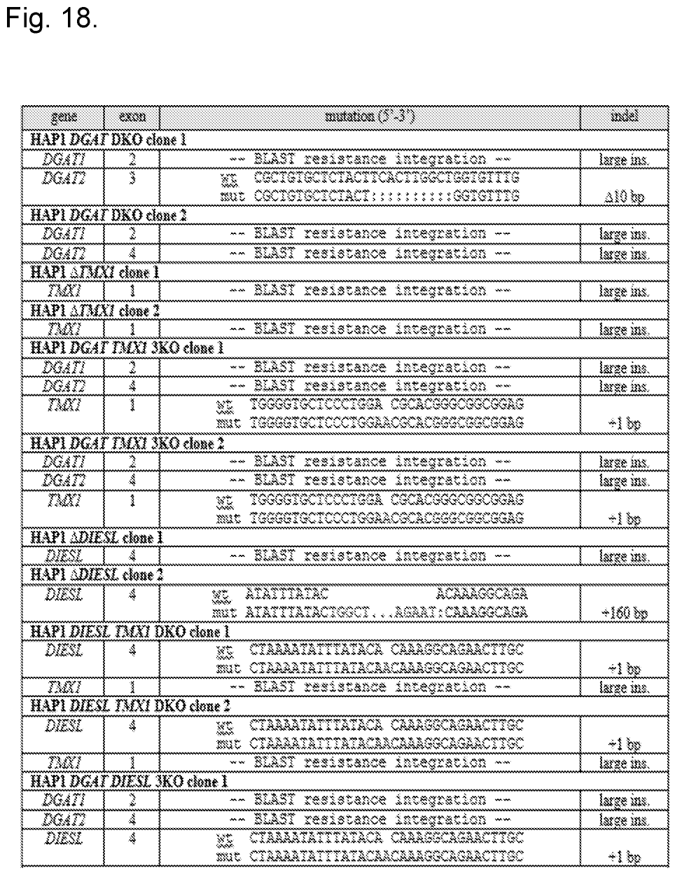

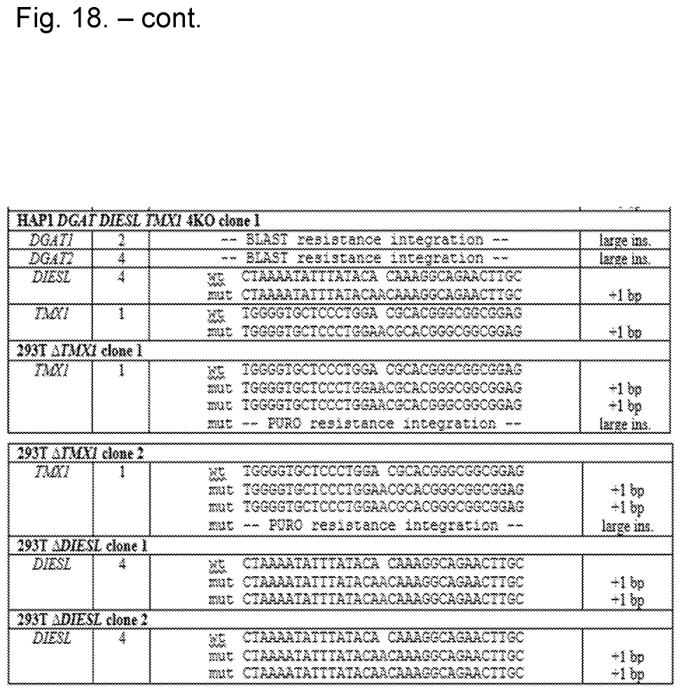

HAP1 and 293T cells were transfected with pX330 encoding the sgRNA of choice, along with a plasmid either carrying an integration cassette (33) or itself encoding a blasticidin or puromycin resistance gene, using Xfect (Takara) according to the manufacturer's instructions. Transfected cells were selected with 25 μg/ml blasticidin (HAP1) or 1 μg/ml puromycin (HAP1 and 293T). After selection, the medium was replaced with complete medium and clones were allowed to grow out to form colonies, which were eventually picked by micropipette and transferred to 24-well plates. Genetic modification of individual clones was detected by PCR, in certain cases using a primer annealing to the sequence of the blasticidin (5′-CCGACATGGTGCTTGTTGTCCTC-3′ (SEQ ID NO:3) or puromycin (5′-GCAACCTCCCCTTCTACGAGC-3′ (SEQ ID NO:4) resistance gene. See FIG. 17 for a list of primers used to amplify genomic loci. Disruption of the locus was confirmed by Sanger sequencing; either directly or, in the case of compound heterozygotes, using TIDE analysis (34) for 293T lines. The targeted genetic modifications within cell lines used in this study are summarized in FIG. 18.

### Lentiviral Transduction

Lentivirus was produced in 293T cells transfected with pAVPR, pVSVg and either pLEX305 or pLentiCRISPRv2 lentiviral plasmids, as well as pAdVAntage. Two days post-transfection, virus was harvested by collecting conditioned medium passed through a 40 μm filter. Viral supernatants, supplemented with 8 μg/ml protamine sulfate, were applied directly to cells for 24h. In certain instances, a second harvest and transduction was performed to improve efficiency. Transduced cells were selected with puromycin (HAP1, 1 μg/ml; A549, 0.5 μg/ml; HeLa, 2 μg/ml; U251, 2 μg/ml; U2OS, 2 μg/ml) or transduction efficiency was assessed by expression of a fluorescent marker (mCherry). RPE1 cells were transduced with a large viral titre and were not selected. Transduced cells were analyzed between 7 and 28 days after transduction.

### Mutagenesis Screening

Haploid genetic screens were performed as described by Brockmann et al. (17). Typically, 2-3×10° gene-trapped HAP1 cells of the indicated genotype were harvested by trypsinization and fixed in Fix Buffer I (BD Biosciences) for 10 minutes at 37° C. For the oleic acid-loaded screen, cells were first cultured for 24h in complete medium supplemented with 200 UM oleic acid, and then chased in medium lacking oleic acid for another 24h prior to harvesting. Cells were treated with 1 mg/ml RNase A (Qiagen) diluted in FACS buffer (10% FBS in PBS) at 37° C. for 30 minutes prior to staining with 1 μg/ml BODIPY 493/503 and 10 μg/ml propidium iodide (Sigma-Aldrich), diluted in FACS buffer, for one hour at room temperature. Cells were washed twice in FACS buffer before being passed through a 40 μm cell strainer. Cell sorting was performed using an S3 Sorter (Bio-Rad), collecting 106 cells (per population) representing the lowest (LO) and highest (HI) 5% of BODIPY signal from haploid cells in G1. The isolation of genomic DNA, preparation of sequencing libraries and their analysis were performed as described previously (17). Reads were aligned to the reference genome (GRCh37 genome assembly), tolerating one mismatch, and sense insertions in the LO and HI populations were compared using a Fisher exact test to determine significant differences (p<. 05). The mutational index (MI) represents the ratio of the occurrence of unique, disruptive (i.e. insertion [ins.] of gene-trap in the sense orientation) mutations in the body of a given gene (5′-UTR, exon and intron) in the HI compared to the LO population, normalized by the total other unique, disruptive mutations in each population (17):

$$\frac{\begin{matrix}¶ ( sense ins . of gene X in HI / ( total sense ins . in HI - \\ sense ins . of gene X in HI ) )\end{matrix}}{\begin{matrix}¶ ( sense inse . of gene X in LO / ( total sense ins . in LO - \\ sense ins . of gene X in LO ) )\end{matrix}} ¶$$

### SDS-PAGE and Immunoblot Analysis

Unless otherwise specified, cells were lysed in RIPA buffer (25 mM Tris-HCl PH 7.5, 150 mM NaCl, 1% NP-40, 1% sodium deoxycholate, 0.1% SDS and protease inhibitor cocktail) on ice. Lysates were sonicated twice (40% amplitude for four seconds), cleared by centrifugation and protein concentrations were determined by BCA assay (Thermo Scientific). Equivalent amounts of protein were separated by SDS-PAGE over Bolt 4-12% Bis-Tris gels (Invitrogen), then transferred to nitrocellulose membranes. Membranes were blocked in 5% milk/TBST, and then incubated overnight at 4° C. in primary antibody diluted in 3% BSA/TBST. The following day, membranes were washed three times in TBST and then incubated in HRP-conjugated secondary antibodies (Invitrogen and Bio-Rad), diluted in blocking buffer, for one hour at room temperature. After three more washes in TBST, signal was developed using Clarity Western ECL substrate (Bio-Rad) and imaged on a Universal Hood II GelDoc system (Bio-Rad).

Recombinant Expression in E. coli

E. coli BL21 were transformed with pET24 or pET28a plasmids expressing the indicated construct. Single clones were cultured up to a 5 ml volume in LB broth at OD 0.5 at 37° C. Cultures were then transferred to 25° C. for one hour, and then expression was induced with 0.2 mM IPTG for 16h. For SDS-PAGE and immunoblot analysis, 100 ul of culture was pelleted for 5 minutes at 3,500 g before lysis in SDS-PAGE sample buffer.

### Thin-Layer Chromatography and NBD-DAG Acylation Assay

Equivalent numbers of human cells grown to confluence in 6-well plates were first washed with PBS. Cells were then incubated twice in 400 μl extraction buffer (3:2 hexane:isopropanol) for 10 minutes (35). E. coli cell pellets from 2 ml of overnight culture were incubated in 500 μl extraction buffer (only once) for 30 minutes while rotating. Extractions were pooled and dried under nitrogen stream, to a volume of ˜10 μl, and then spotted on silica gel 60 TLC plates (Sigma-Aldrich). Plates were placed in a TLC tank containing a mobile phase to separate neutral lipids (80:20:1 hexane:diethyl ether:acetic acid) or polar lipids (60:50:4:1 chloroform:methanol:water:acetic acid). After separation, plates were air-dried and then stained with 0.2% Amido Black 10B (Sigma-Aldrich) dissolved in 1 M NaCl for 15 to 30 minutes (36). After staining, plates were rinsed with water and then washed several times with 1 M NaCl prior to drying overnight. Plates were imaged the following day on a Universal Hood II or EZ Imager GelDoc system (Bio-Rad). For the acylation of NBD-DAG, unless otherwise specified, 25 μM NBD-DAG (Avanti Polar Lipids) was first conjugated to 0.125% fatty acid-free BSA in serum-free IMDM for one hour at 37° C. Cells were then incubated in this medium for one hour prior to lipid extraction. TLC was carried out as above. After drying, NBD fluorescence was monitored on a Universal Hood GelDoc system (Bio-Rad) using the fluorescein channel. For cell-free [14C] DAG acylation, HAP1 4KO (naïve or expressing 3xHA-DIESL) were washed in ice-cold PBS, collected in ice-cold buffer (20 mM HEPES pH 7.4, 250 mM sucrose, 2 mM MgCl2) and lysed by passing through a glass homogenizer sixty times on ice. Lysates were cleared by centrifugation at 600 g for 10 minutes at 4° C.; protein content was assayed and lysates were diluted to 2 mg/ml and stored at −80° C. Reconstitution assays were based on assays described by Gaebler et al. (45), and were performed at the Radionuclide Centre (RNC) of the NKI under RNC guidelines. First, 1.4 μl of 1.8 mM [14C] DAG, 3.6 μl buffer and 5.0 μl of 6.6 mg/ml fatty-acid free BSA were incubated on a heat block for one hour at 37° C., shaking at 850 rpm. To this was added 100 μl of the indicated lysate and assays were incubated at 37° C. (shaking at 850 rpm) for the indicated time (final [14C] DAG concentration, 50 μM; total radioactivity per assay, 0.14 μCi). For certain controls, the reaction was incubated instead on wet ice, or the lysate was heat-inactivate for 20 minutes at 90° C. prior to addition to the reaction. Reactions were quenched by performing a modified Bligh and Dyer extraction (46); 110 μl chloroform and 110 μl methanol were added to each reaction, mixed and centrifuged at 20,000 g for two minutes at room-temperature. The bottom organic layer was transferred to a new tube, dried and reconstituted in 8 μl ethanol and separated by neutral lipid TLC as above. Plates were dried and imaged on a Typhoon FLA 9500 phosphorimager (General Electric) after exposing to a BAS-TR2949 imaging plate (Fuji) for three days.

### Immunoprecipitation and Crosslinking

### Formaldehyde Crosslinking

HAP1 ΔDIESL and ATMX1ΔDIESL cells reconstituted with 3xHA-DIESL, or HeLa WT cells expressing 3xHA-DIESL, were washed with PBS and incubated in 1% paraformaldehyde for 20 minutes at room temperature. Cross-linking reactions were quenched by incubation in 0.2 M glycine for 10 minutes. Cells were then lysed in RIPA buffer on ice as described above, sonicated and protein concentrations were determined. Samples were separated by SDS-PAGE and immunoblotted as described above.

### Co-Immunoprecipitation

HAP1 ΔDIESL cells reconstituted with 3xHA-DIESL, grown to confluence in a 6-well plate, were lysed in 400 μl IP buffer (25 mM HEPES PH 7.0, 150 mM NaCl, 1% detergent [Tween-20 unless otherwise indicated], and protease/phosphatase inhibitor cocktails) on ice, sonicated and cleared by centrifugation. 250 μl of lysate was incubated with 10 μl of PBS-washed anti-HA magnetic beads (Pierce) overnight while rotating at 4° C. The following day, beads were washed three times in IP buffer before elution in SDS-PAGE sample buffer at 90° C. for 20 minutes. One-quarter of this eluate (along with the corresponding amount of input sample) were separated by SDS-PAGE and subjected to immunoblot analysis as above.

### Deglycosylation Assay

HAP1 ΔDIESL cells reconstituted with 3xHA-DIESL were lysed in buffer (25 mM Tris-HCl PH 7.5, 150 mM NaCl, 1% NP-40, protease inhibitor cocktail) on ice, sonicated and protein levels were quantified as described above. N-glycans were removed using PNGase F (New England BioLabs) according to the manufacturer's instructions. Denatured lysates (1 μg/μl) were incubated for 15 minutes at 37° C. while shaking, in the presence or absence of 25 units/μl PNGase F. Reactions were separated by SDS-PAGE and analyzed by immunoblot as described above.

### Protease Protection Assay

### Crude ER Isolation

8×150 mm plates of confluent HAP1 ΔDIESL cells, reconstituted with 3xHA-DIESL, were collected in 1 ml isolation buffer (20 mM HEPES PH 7.2, 220 mM mannitol, 68 mM sucrose, 80 mM KCl, 2 mM MgCl2, protease inhibitor cocktail) per plate on ice and passed through 18- and 25-gauge needles ten times each. Lysates were centrifuged for 10 minutes at 1,000 g at 4° C. The post-nuclear supernatant was cleared of heavy membranes by centrifugation at 10,000 g for 15 minutes at 4° C. Crude microsomes were isolated from the resulting supernatant by centrifugation at 100,000 g for 30 minutes at 4° C. Microsomal pellets were resuspended in isolation buffer and the protein was quantified by BCA assay (Thermo Scientific) as above.

### Protease Protection Assay

Isolated microsomes (5 mg/ml) were incubated in increasing amounts (0 to 100 μg/ml) of Proteinase K from Tritirachium album (Sigma-Aldrich) for 30 minutes at 37° C. while shaking. Reactions were quenched with an equal volume of SDS-PAGE sample buffer. Reactions were separated by SDS-PAGE and analyzed by immunoblot as described above.

### Subcellular Fractionation and MAM Purification

The mitochondria-associated membrane of the ER (MAM) was purified from six confluent 15 cm plates of HAP1 cells according to the method by Lewis et al. (47), with all steps performed on ice or at 4° C. Cells were washed in ice-cold PBS, collected in 750 μl/plate ice-cold homogenization buffer (10 mM HEPES pH 7.4, 250 mM sucrose) and passed through a glass homogenizer 100 times. The homogenate was centrifuged for 5 minutes at 600 g. The pellet was resuspended in 2 ml homogenization buffer, homogenized and centrifuged again. Both supernatants were pooled and centrifuged at 10,300 g for 20 minutes. The supernatant was put aside and the pellet (crude mitochondria) was resuspended in 1 ml isolation buffer 1 (5 mM HEPES pH 7.4, 250 mM mannitol, 0.5 mM EGTA). 500 μl of resuspended mitochondrial pellet was layered on top of 1.5 ml Percoll solution (25 mM HEPES PH 7.4, 225 mM mannitol, 1 mM EGTA, 30% [v/v] Percoll) in two ultracentrifuge tubes. Mitochondria were fractionated by centrifugation at 95,000 g for 30 minutes using a TLS-55 swinging-bucket rotor (Beckman). Pure mitochondria and crude MAM layers were collected using a syringe, and resuspended in 4 volumes isolation buffer 1 and isolation buffer 2 (25 mM HEPES pH 7.4, 225 mM mannitol, 1 mM EGTA), respectively. Purified mitochondria were centrifuged at 10,500 g for 10 minutes. The crude MAM was cleared by centrifugation (6,300 g for 10 minutes), and then it and the post-mitochondrial supernatant from earlier were both centrifuged for one hour at 100,000 g. The pellets (microsome and pure MAM) were collected and the cytosolic supernatant was concentrated by centrifugal filtration (10 kDa filter, Amicon). Protein concentration was determined by BCA assay (Thermo Scientific) and fractions were stored at −80° C. prior to immunoblot analysis.

### Electron Microscopy

Preparation of cells for transmission electron microscopy was performed as previously described (37). Grids were imaged on a Tecnai 12 G2 (ThermoFisher).

### Immunofluorescence and Confocal Microscopy

Cells were grown on glass coverslips and fixed in 4% formaldehyde in PBS for 15 minutes at room temperature, and then washed three times in PBS. Cells were permeabilized with 0.1% Triton X-100 in PBS for 10 minutes, then washed three times in PBS and blocked in 1% BSA in PBS for 20 minutes. Cells were stained with primary antibodies, diluted in blocking buffer, for one hour at room temperature. After three washes in blocking buffer, cells were stained with Alexa Fluor-conjugated secondary antibodies (Invitrogen) and/or neutral lipid dyes, diluted in blocking buffer, for one hour at room temperature. Cells were washed three times in PBS, counterstained with Hoechst 33342 (Invitrogen) diluted in PBS, and then washed three more times in PBS prior to mounting on glass slides using Aqua Poly/Mount (Polysciences Inc.). Cells were imaged by confocal laser-scanning microscopy on a Leica SP5 microscope using a 60X (1.4 NA) objective. Images were analyzed using ImageJ (NIH).

### Flow Cytometry

For BODIPY 493/503 measurements in fixed HAP1 cells, HAP1 cells, grown in 10 cm plates, were collected by trypsinization and were fixed in Fix Buffer I (BD Biosciences) for 10 minutes at 37° C. Cells were pelleted, washed with FACS buffer (10% FBS in PBS), resuspended in FACS buffer and counted. 10 million cells were stained with 1 μg/ml BODIPY 493/503 and 5 μg/ml DAPI (Invitrogen), diluted in FACS buffer, for one hour at room temperature. Cells were washed once in FACS buffer, then passed through a 35 μm nylon mesh cell strainer into a FACS tube. Fluorescence was analyzed on an LSR Fortessa (BD Biosciences) analytical flow cytometer, using 405 and 488 nm lasers to detect DAPI and BODIPY 493/503, respectively. Data were analyzed using FlowJo (BD Life Sciences). Fluorescence plots represent the fluorescent signal measured in single haploid G1 cells, as determined by DAPI fluorescence intensity. For mitochondrial measurements in live RPE1 cells, RPE1 cells were treated as described and pulsed for 30 minutes with either 600 nM TMRM or 250 nM MitoTracker Red CM-H2XROS in medium depleted of lipoproteins. In TMRM experiments, cells were then incubated in 150 mM for an additional 30 minutes, using 20 UM CCCP as a positive control. Cells were collected by trypsinization and stored on ice in FACS buffer. Fluorescence (using a 561 nm laser) and data were analyzed as above. Fluorescence plots represent the fluorescent signal measured in single cells. Membrane potential was calculated by subtracting the fluorescence mean of depolarized (CCCP-treated) cells.

### ATP Measurements

RPE1 cells were plated in 96-well plates and ATP levels were measured using CellTitre-Glo (Promega) according to the manufacturer's instructions. ATP levels were normalized by measuring protein content in a parallel plate by BCA assay (Thermo Scientific).

### Mice

The Animal Ethics Committee of the NKI approved all animal experiments, which were implemented in accordance with institutional, national, and European guidelines for Animal Care and Use. Frozen embryos (morula stage, from C57/BL6J mice) carrying a single disruptive allele in Diesl/Tmem68 (tm1a [EUCOMM] Wtsi, herein referred to as gt [lacZ-neo]) were purchased from EUCOMM. After thawing, embryos were developed into blastocysts overnight in KSOM medium in an incubator at 37° C., and were then implanted into C57/BL6N foster females and carried to term. Mice were genotyped by PCR, using primer combinations to detect the WT (5′-GCTCCCTTCCATTTACTCTG-3′ (SEQ ID NO: 5) and 5′-CCGGTGAGATAGCTAACAAG-3′ (SEQ ID NO: 6)) and mutant (5′-CTTATCATGTCTGGATCCGG-3′ (SEQ ID NO: 7) and 5′-CCGGTGAGATAGCTAACAAG-3′ (SEQ ID NO: 8)) alleles. As 6J mice harbor a deletion in the Nnt gene that influences DIESL protein levels (27), only mice with 6N alleles at the Nnt locus were used in this study and this was monitored by PCR, using primer combinations to detect the 6N (5′-GGGCATAGGAAGCAAATACCAAGTTG-3′ (SEQ ID NO: 9) and 5′-GTAGGGCCAACTGTTTCTGCATGA-3′ (SEQ ID NO: 10)) and 6J (5′-GTGGAATTCCGCTGAGAGAACTCTT-3′ (SEQ ID NO: 11) and 5′-GTAGGGCCAACTGTTTCTGCATGA-3′ (SEQ ID NO: 12)) alleles. For histological analysis, tissues and organs were collected and fixed in EAF fixative (ethanol/acetic acid/formaldehyde/saline at 40:5:10:45 v/v) and embedded in paraffin. Sections were prepared at 2 μm thickness from the paraffin blocks and stained with hematoxylin and eosin (HE) according to standard procedures. The sections were reviewed with a Zeiss Axioskop2 Plus microscope (Carl Zeiss Microscopy, Jena, Germany) and images were captured with a Zeiss AxioCam HRc digital camera and processed with AxioVision 4 software (both from Carl Zeiss Vision, München, Germany).

### Shotgun Lipidomics

### Sample Preparation

HAP1 4KO (ΔDGAT1ΔDGAT2ΔDIESLΔTMX1) cells, reconstituted with WT or H130A DIESL, were cultured in complete medium. Prior to collection, cells were cultured in serum-free IMDM for thirty minutes. Cells were then collected by trypsinization, washed twice with PBS and three million cells (in triplicate) were pelleted and stored at −80° C. For E. coli samples, 2 ml of induced culture (as described above, in triplicate) was pelleted and incubated overnight at −80° C. The following day, bacterial pellets were thawed and incubated in 25 μg/ml lysozyme in PBS for 30 minutes. Cells were pelleted, washed in PBS, and then pelleted again before resuspension in distilled water. Cells were sonicated at 60% amplitude for 5 minutes, in continuous 5-second on/25-second off cycles. The insoluble fraction was pelleted by centrifugation and the lysate (supernatant) was quantified for protein and stored at −80° C. For mouse tissue samples, tissue stored at −80° C. was thawed on ice, weighed and homogenized in PBS (50 mg/ml tissue) using a glass homogenizer with 20 and 40 strokes for brain and liver tissue, respectively. Homogenates were diluted 10-fold further in PBS and homogenized again with 20 strokes. Homogenates (5 mg/ml) were stored at −80° C.

### Lipid Extraction for Mass Spectrometry Lipidomics

Mass spectrometry-based lipid analysis was performed by Lipotype GmbH (Dresden, Germany) as described (38). Lipids were extracted using a two-step chloroform/methanol procedure (39). Samples were spiked with internal lipid standard mixture containing: cardiolipin 14:0/14:0/14:0/14:0 (CL), ceramide 18:1;2/17:0 (Cer), diacylglycerol 17:0/17:0 (DAG), hexosylceramide 18:1;2/12:0 (HexCer), lyso-phosphatidate 17:0 (LPA), lyso-phosphatidylcholine 12:0 (LPC), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG), phosphatidylinositol 16:0/16:0 (PI), phosphatidylserine 17:0/17:0 (PS), cholesterol ester 20:0 (CE), sphingomyelin 18:1;2/12:0;0 (SM) and triacylglycerol 17:0/17:0/17:0 (TAG). After extraction, the organic phase was transferred to an infusion plate and dried in a speed vacuum concentrator. 1st step dry extract was re-suspended in 7.5 mM ammonium acetate in chloroform/methanol/propanol (1:2:4, V:V:V) and 2nd step dry extract in 33% ethanol solution of methylamine in chloroform/methanol (0.003:5:1; V:V:V). All liquid handling steps were performed using Hamilton Robotics STARlet robotic platform with the Anti Droplet Control feature for organic solvents pipetting.

### MS Data Acquisition

Samples were analyzed by direct infusion on a QExactive mass spectrometer (Thermo Scientific) equipped with a TriVersa NanoMate ion source (Advion Biosciences). Samples were analyzed in both positive and negative ion modes with a resolution of Rm/z=200=280000 for MS and Rm/z=200=17500 for MSMS experiments, in a single acquisition. MSMS was triggered by an inclusion list encompassing corresponding MS mass ranges scanned in 1 Da increments (40). Both MS and MSMS data were combined to monitor CE, DAG and TAG ions as ammonium adducts; PC, PC O—, as acetate adducts; and CL, PA, PE, PE O—, PG, PI and PS as deprotonated anions. MS only was used to monitor LPA, LPE, LPE O—, LPI and LPS as deprotonated anions; Cer, HexCer, SM, LPC and LPC O- as acetate adducts.

### Data Analysis and Post-Processing

Data were analyzed by Lipotype GmbH with in-house developed lipid identification software based on LipidXplorer (41, 42). Data post-processing and normalization were performed using an in-house developed data management system. Only lipid identifications with a signal-to-noise ratio >5, and a signal intensity 5-fold higher than in corresponding blank samples were considered for further data analysis.

### Homology Modeling and Data Analysis

Homology modeling, using full-length human DIESL (Q96MH6-1) as an input sequence, was performed using the PHYRE2 (http://www.sbg.bio.ic.ac.uk/˜phyre2) and iTASSER (https://zhanglab.dcmb.med.umich.edu/l-TASSER/) web portals (43, 44). The PlsC structure and DIESL homology models were visualized using PyMOL. Data wrangling, statistical analyses and plot generation was performed with Prism (GraphPad Software) and RStudio.

### REFERENCES

1. G. I. Shulman, Ectopic fat in insulin resistance, dyslipidemia, and cardiometabolic disease. N Engl J Med 371, 2237-2238 (2014).
2. O. Quehenberger, E. A. Dennis, The human plasma lipidome. N Engl J Med 365, 1812-1823 (2011).
3. S. K. Das, G. Hoefler, The role of triglyceride lipases in cancer associated cachexia. Trends Mol Med 19, 292-301 (2013).
4. S. Cases et al., Identification of a gene encoding an acyl CoA: diacylglycerol acyltransferase, a key enzyme in triacylglycerol synthesis. Proceedings of the National Academy of Sciences of the United States of America 95, 13018-13023 (1998).
5. S. J. Smith et al., Obesity resistance and multiple mechanisms of triglyceride synthesis in mice lacking Dgat. Nature genetics 25, 87-90 (2000).
6. E. Herker, G. Vieyres, M. Beller, N. Krahmer, M. Bohnert, Lipid Droplet Contact Sites in Health and Disease. Trends in cell biology, (2021).
7. S. B. Weiss, E. P. Kennedy, J. Y. Kiyasu, The enzymatic synthesis of triglycerides. The Journal of biological chemistry 235, 40-44 (1960).
8. T. C. Walther, J. Chung, R. V. Farese, Jr., Lipid Droplet Biogenesis. Annu Rev Cell Dev Biol 33, 491-510 (2017).
9. X. Sui et al., Structure and catalytic mechanism of a human triacylglycerol-synthesis enzyme. Nature 581, 323-328 (2020).
10. L. Wang et al., Structure and mechanism of human diacylglycerol O-acyltransferase 1. Nature 581, 329-332 (2020).
11. A. M. Birch, L. K. Buckett, A. V. Turnbull, DGAT1 inhibitors as anti-obesity and anti-diabetic agents. Curr Opin Drug Discov Devel 13, 489-496 (2010).
12. C. L. Yen, D. W. Nelson, M. I. Yen, Intestinal triacylglycerol synthesis in fat absorption and systemic energy metabolism. J Lipid Res 56, 489-501 (2015).
13. N. L. Gluchowski et al., Hepatocyte Deletion of Triglyceride-Synthesis Enzyme Acyl CoA: Diacylglycerol Acyltransferase 2 Reduces Steatosis Without Increasing Inflammation or Fibrosis in Mice. Hepatology 70, 1972-1985 (2019).
14. R. Loomba et al., Novel antisense inhibition of diacylglycerol O-acyltransferase 2 for treatment of non-alcoholic fatty liver disease: a multicentre, double-blind, randomised, placebo-controlled phase 2 trial. Lancet Gastroenterol Hepatol 5, 829-838 (2020).
15. J. Jacquemyn, A. Cascalho, R. E. Goodchild, The ins and outs of endoplasmic reticulum-controlled lipid biosynthesis. EMBO reports 18, 1905-1921 (2017).
16. C. A. Harris et al., DGAT enzymes are required for triacylglycerol synthesis and lipid droplets in adipocytes. J Lipid Res 52, 657-667 (2011).
17. M. Brockmann et al., Genetic wiring maps of single-cell protein states reveal an off-switch for GPCR signalling. Nature 546, 307-311 (2017).
18. Y. Matsuo et al., Identification of a novel thioredoxin-related transmembrane protein. The Journal of biological chemistry 276, 10032-10038 (2001).
19. P. Chang et al., Molecular identification of transmembrane protein 68 as an endoplasmic reticulum-anchored and brain-specific protein. PloS one 12, e0176980 (2017).
20. J. Lippincott-Schwartz, L. C. Yuan, J. S. Bonifacino, R. D. Klausner, Rapid redistribution of Golgi proteins into the ER in cells treated with brefeldin A: evidence for membrane cycling from Golgi to ER. Cell 56, 801-813 (1989).
21. R. M. Robertson et al., A two-helix motif positions the lysophosphatidic acid acyltransferase active site for catalysis within the membrane bilayer. Nature structural & molecular biology 24, 666-671 (2017).
22. J. Rucker, J. Paul, B. A. Pfeifer, K. Lee, Engineering E. coli for triglyceride accumulation through native and heterologous metabolic reactions. Appl Microbiol Biotechnol 97, 2753-2759 (2013).
23. International Mouse Phenotyping Consortium (https://www.mousephenotype.org/data/charts?accession=MGI: 1919348&allele ac cession id=MGI: 4431978&zygosity=homozygote&parameter stable id=ESLIM 0 08 001 014&pipeline stable_id=ESLIM 002&procedure stable_id=ESLIM 008 001&parameter stable id=ESLIM 008 001 014&phenotyping center=ICS), accessed Jun. 11, 2021.
24. A. Raturi et al., TMX1 determines cancer cell metabolism as a thiol-based modulator of ER-mitochondria Ca2+ flux. The Journal of cell biology 214, 433-444 (2016).
25. X. Zhang et al., Redox signals at the ER-mitochondria interface control melanoma progression. The EMBO journal 38, e100871 (2019).
26. A. A. Toye et al., A genetic and physiological study of impaired glucose homeostasis control in C57BL/6J mice. Diabetologia 48, 675-686 (2005).
27. J. M. Chick et al., Defining the consequences of genetic variation on a proteome-wide scale. Nature 534, 500-505 (2016).
28. A. Ali et al., Genome-wide scan for common variants associated with intramuscular fat and moisture content in rainbow trout. BMC Genomics 21, 529 (2020).
29. J. D. Leal-Gutierrez et al., Whole Genome Sequence Data Provides Novel Insights Into the Genetic Architecture of Meat Quality Traits in Beef. Front Genet 11, 538640 (2020).
30. A. F. Magalhaes et al., Genome-Wide Association Study of Meat Quality Traits in Nellore Cattle. PloS one 11, e0157845 (2016).
31. M. S. Brown, J. L. Goldstein, A receptor-mediated pathway for cholesterol homeostasis. Science 232, 34-47 (1986).
32. M. S. Brown, A. Radhakrishnan, J. L. Goldstein, Retrospective on Cholesterol Homeostasis: The Central Role of Scap. Annu Rev Biochem 87, 783-807 (2018). D. H. Lackner et al., A generic strategy for CRISPR-Cas9-mediated gene tagging. Nature communications 6, 10237 (2015).
34. E. K. Brinkman, T. Chen, M. Amendola, B. van Steensel, Easy quantitative assessment of genome editing by sequence trace decomposition. Nucleic Acids Res 42, e168 (2014).
35. J. Chung et al., LDAF1 and Seipin Form a Lipid Droplet Assembly Complex. Developmental cell 51, 551-563 e557 (2019).
36. A. Y. Plekhanov, Rapid staining of lipids on thin-layer chromatograms with amido black 10B and other water-soluble stains. Anal Biochem 271, 186-187 (1999).
37. A. Zuidema et al., Mechanisms of integrin alphaVbeta5 clustering in flat clathrin lattices. Journal of cell science 131, (2018).
38. J. L. Sampaio et al., Membrane lipidome of an epithelial cell line. Proceedings of the National Academy of Sciences of the United States of America 108, 1903-1907 (2011).
39. C. S. Ejsing et al., Global analysis of the yeast lipidome by quantitative shotgun mass spectrometry. Proceedings of the National Academy of Sciences of the United States of America 106, 2136-2141 (2009).
40. M. A. Surma et al., An automated shotgun lipidomics platform for high throughput, comprehensive, and quantitative analysis of blood plasma intact lipids. Eur J Lipid Sci Technol 117, 1540-1549 (2015).
41. R. Herzog et al., LipidXplorer: a software for consensual cross-platform lipidomics. PloS one 7, e29851 (2012).
42. R. Herzog et al., A novel informatics concept for high-throughput shotgun lipidomics based on the molecular fragmentation query language. Genome Biol 12, R8 (2011).
43. L. A. Kelley, S. Mezulis, C. M. Yates, M. N. Wass, M. J. Sternberg, The Phyre2 web portal for protein modeling, prediction and analysis. Nature protocols 10, 845-858 (2015).
44. A. Roy, A. Kucukural, Y. Zhang, I-TASSER: a unified platform for automated protein structure and function prediction. Nature protocols 5, 725-738 (2010).
45 Gaebler, A. et al. Alkyne lipids as substrates for click chemistry-based in vitro enzymatic assays. J. Lipid Res. 54, 2282-2290 (2013).
46. Bligh, E. G. & Dyer, W. J. A rapid method of total lipid extraction and purification. Can. J. Biochem. Physiol. 37, 911-917 (1959).
47. Lewis, A., Tsai, S.-Y. & Su, T.-P. Detection of Isolated Mitochondria-Associated ER Membranes Using the Sigma-1 Receptor. Methods Mol. Biol. Clifton NJ 1376, 133-140 (2016).
48. Chikka, M. R., McCabe, D. D., Tyra, H. M. & Rutkowski, D. T. C/EBP homologous protein (CHOP) contributes to suppression of metabolic genes during endoplasmic reticulum stress in the liver. J. Biol. Chem. 288, 4405-4415 (2013).
49. DeZwaan-McCabe, D. et al. The stress-regulated transcription factor CHOP promotes hepatic inflammatory gene expression, fibrosis, and oncogenesis. PLoS Genet. 9, e1003937 (2013).
50. Trefts, E. & Shaw, R. J. AMPK: restoring metabolic homeostasis over space and time. Mol. Cell 81, 3677-3690 (2021).
51. Oakhill, J. S. et al. AMPK is a direct adenylate charge-regulated protein kinase. Science 332, 1433-1435 (2011).
52. Listenberger, L. L. et al. Triglyceride accumulation protects against fatty acid-induced lipotoxicity. Proc. Natl. Acad. Sci. U.S.A 100, 3077-3082 (2003).
53. Chitraju, C., Walther, T. C. & Farese, R. V. The triglyceride synthesis enzymes DGAT1 and DGAT2 have distinct and overlapping functions in adipocytes. J. Lipid Res. 60, 1112-1120 (2019).
54. van Rijn, J. M. et al. DGAT2 partially compensates for lipid-induced ER stress in human DGAT1-deficient intestinal stem cells. J. Lipid Res. 60, 1787-1800 (2019).
55. Ronchi, J. A. et al. A spontaneus mutation in the nicotinamide nucleotide transhydrogenase gene of C57BL/6J mice results in mitochondrial redox abnormalities. Free Radical Biology and Medicine. 63, 446-456 (2013).

1. An isolated DGAT1/2-Independent Enzyme Synthesizing storage Lipids (DIESL) polypeptide, wherein the DIESL polypeptide comprises an amino acid sequence that has at least 75% amino acid sequence identity with the amino acid sequence as defined in SEQ ID NO:1, wherein the DIESL polypeptide exhibits triacylglycerol synthase activity.

2. The DIESL polypeptide according to claim 1 wherein the polypeptide comprises an amino acid sequence having at least 90% sequence identity to SEQ ID NO:1, having at least 95% sequence identity to SEQ ID NO:1, having at least 98% sequence identity to SEQ ID NO:1, having at least 99% sequence identity to SEQ ID NO:1, having the amino acid sequence as defined in SEQ ID NO:1, or is transmembrane protein 68.

3. An isolated DIESL polynucleotide, wherein the polynucleotide encodes for the DIESL polypeptide according to any of claims 1-2.

4. A vector comprising the DIESL polynucleotide according to claim 3.

5. A host cell comprising the DIESL polypeptide, the DIESL polynucleotide and/or the vector according to any of claims 1-4.

6. Use of a DIESL polypeptide in the production of triacylglycerol.

7. The use according to claim 6 wherein the DIESL polypeptide exhibits triacylglycerol synthase activity.

8. The use according to any of claims 6-7 wherein the DIESL polypeptide according to any of claims 1-2 and/or a DIESL polynucleotide that encodes the DIESL polypeptide is comprised in a cell.

9. The use according to any of claims 6-8 wherein the cell is selected from the group consisting off a prokaryotic cell, an eukaryotic cell, a bacterium, a yeast cell, a fungus cell, an animal cell, a plant cell, a cell that naturally lacks the ability to produce triacylglycerol, a cell that does not express a further diglyceride acyltransferase, preferably a cell that does not express a Diacylglycerol O-acyltransferase 1 (DGAT1) polypeptide and/or a Diacylglycerol O-acyltransferase 2 (DGAT2) polypeptide, a cell wherein the expression of a DGAT1 polypeptide and/or a DGAT2 polypeptide is modified, a cell that does not express a Thioredoxin-related transmembrane protein 1 (TMX1) polypeptide, a cell wherein the expression of a TMX1 polypeptide is modified, a cell that does not express a DGAT1 polypeptide, a DGAT2 polypeptide, and/or a TMX1 polypeptide, or a cell that overexpresses the DIESL polypeptide.

10. The use of the DIESL polypeptide according to any of claims 6-9 wherein the use further comprises obtaining or purifying the triacylglycerol produced.

11. A screening method for identifying an agent that modulates the activity of a DIESL polypeptide, wherein the method comprises
(a) contacting the DIESL polypeptide with a candidate agent; and
(b) detecting a change in activity of the DIESL polypeptide compared to a control to determine the candidate agent's modulatory activity.

12. The screening method according to claim 11 wherein the method comprises introducing the candidate agent in a cell that comprises the DIESL polypeptide and/or that comprises a DIESL polynucleotide that encodes the DIESL polypeptide.

13. The screening method according to any of claims 11-12 wherein detecting a change in activity comprises detecting triacylglycerol synthase activity, triacylglycerol production, triacylglycerol accumulation, incorporation of a fatty acid into a diacylglycerol, and/or formation of lipid droplet organelles.

14. The screening method according to any of claims 11-13 wherein the method comprises
(a) providing a cell that expresses the DIESL polypeptide and providing a candidate agent;
(b) contacting the candidate agent with the cell that expresses the DIESL polypeptide under conditions that allow the cell to produce triacylglycerol;
(c) comparing the activity of the DIESL polypeptide, preferably comparing triacylglycerol synthase activity, triacylglycerol production, triacylglycerol accumulation, incorporation of a fatty acid into a diacylglycerol, and/or formation of lipid droplet organelles to a control;
(d) identifying agents that provide for an increase or decrease in the activity of the DIESL polypeptide, preferably in triacylglycerol synthase activity, triacylglycerol production, triacylglycerol accumulation, incorporation of a fatty acid into a diacylglycerol, and/or formation of lipid droplet organelles.

15. The screening method according to any of claims 11-14 wherein the cell
does not express a further diglyceride acyltransferase, preferably does not express a DGAT1 polypeptide, a DGAT2 polypeptide and/or a DGAT1 polypeptide and a DGAT2 polypeptide; and/or
does not express a TMX1 polypeptide.

16. The screening method according to any of claims 11-15 wherein the candidate agent is a modulator of a further diglyceride acyltransferase, preferably a DGAT1 polypeptide, a DGAT2 polypeptide and/or a DGAT1 polypeptide and a DGAT2 polypeptide.

17. A screening method for identifying an agent that modulates interaction of a DIESL polypeptide with a Thioredoxin-related transmembrane protein 1 (TMX1) polypeptide, wherein the method comprises:
(a) providing a cell that expresses the DIESL polypeptide and the TMX1 polypeptide;
(b) providing a candidate agent;
(c) contacting the candidate agent with the cell that expresses the DIESL polypeptide and the TMX1 polypeptide under conditions that allow interaction of the candidate agent with the DIESL polypeptide and/or the TMX-1 polypeptide;
(d) determining the amount of DIESL monomer, TMX-1 monomer and/or the amount of DIESL/TMX-1 dimer;
(e) comparing the amounts obtained in step (d) to a control to determine the candidate agent's modulatory activity.

18. The screening method according to claim 17, wherein the amount of DIESL/TMX1 dimer is determined by
crosslinking the cells with PFA, lysing the cells and determine the amount of crosslinked dimer formed;
lysing the cells, immunoprecipitate using an antibody binding for DIESL or TMX1 and determine the presence of the amount of TMX1 or DIESL, respectively in the immunoprecipitate; and/or
lysing the cells, immunoprecipitate using an antibody binding for DIESL or TMX1 and determine the presence of the amount of TMX1 and DIESL in the immunoprecipitate.

19. A screening method for identifying an agent that modulates the activity of a further diglyceride acyltransferase, preferably of DGAT1 and/or DGAT2, wherein the method comprises
(a) contacting the further diglyceride acyltransferase with a candidate agent; and
(b) detecting a change in activity of the further diglyceride acyltransferase compared to a control to determine the candidate agent's modulatory activity on the further diglyceride acyltransferase, and wherein
the further diglyceride acyltransferase is expressed in a cell that does not express a DIESL polypeptide; and/or
wherein the method further comprises performing the screening method according to any of claims 11-16 to determine the candidate agent's modulatory activity on a DIESL polypeptide.

20. A screening method for identifying mutants of a DIESL polypeptide, wherein said mutant has altered activity and/or altered substrate preference, wherein the method comprises
(a) providing a mutant of the DIESL polypeptide; and
(b) detecting activity of the mutant of the DIESL polypeptide compared to a control and/or detecting substrate preference of the mutant of the DIESL polypeptide compared to a control to determine the mutant of the DIESL polypeptide's altered activity and/or substrate preference.

21. A method for producing a pharmaceutical composition comprising a screening method according to any of claims 11-19 and furthermore mixing the agent identified, or a derivative or homologue thereof, with a pharmaceutically acceptable carrier.

22. A method for producing triacylglycerol, wherein the method comprises contacting a DIESL polypeptide with a diglyceride and a source of fatty acids under conditions sufficient for the triacylglycerol to be produced, preferably wherein the DIESL polypeptide is expressed in a cell.

23. The method according to claim 22 wherein the source of fatty acids is selected from the group consisting of fatty acids, an endogenous source of fatty acids, or phospholipids.

24. The method according to any of claims 22-23 wherein the cell is selected from the group consisting off a prokaryotic cell, an eukaryotic cell, a bacterium, a yeast cell, a fungus cell, an animal cell, a plant cell, a cell that naturally lacks the ability to produce triacylglycerol, a cell that does not express a further diglyceride acyltransferase, preferably a cell that does not express a Diacylglycerol O-acyltransferase 1 (DGAT1) polypeptide and/or a Diacylglycerol O-acyltransferase 2 (DGAT2) polypeptide, a cell wherein the expression of a DGAT1 polypeptide and/or a DGAT2 polypeptide is modified, a cell that does not express a Thioredoxin-related transmembrane protein 1 (TMX1) polypeptide, a cell wherein the expression of a TMX1 polypeptide is modified, a cell that does not express a DGAT1 polypeptide, a DGAT2 polypeptide, and/or a TMX1 polypeptide, or a cell that overexpresses the DIESL polypeptide.

25. The method according to any of claims 22-24 wherein the method further comprises obtaining or purifying the triacylglycerol produced.

26. A method for determining triacylglycerol production and/or DIESL activity in a cell wherein the method comprises determining DIESL polypeptide and/or TMX1 polypeptide expression, DIESL polypeptide and/or TMX1 polypeptide level, amount of DIESL/TMX1 dimer, and/or DIESL polypeptide and/or TMX1 polypeptide activity in the cell.

27. The method according to claim 26 wherein the cell is a human cell, wherein the cells is obtained from a subject, preferably a human subject, wherein the cell is obtained from healthy or diseased tissue, and/or wherein the cell is obtained from a tumor.

28. An isolated Thioredoxin-related transmembrane protein 1 (TMX1) polypeptide, wherein the TMX1 polypeptide comprises an amino acid sequence that has at least 75% amino acid sequence identity with the amino acid sequence as defined in SEQ ID NO:2, wherein the TMX1 polypeptide exhibits inhibition of DIESL mediated triacylglycerol synthase activity, TAG production and/or TAG accumulation.

29. An isolated TMX1 polynucleotide, wherein the polynucleotide encodes for the TMX1 polypeptide according claim 28.

30. A vector comprising the TMX1 polynucleotide according to claim 29.

31. A host cell comprising the TMX1 polypeptide, the TMX1 polynucleotide and/or the vector according to any of claims 28-30.

32. Use of a TMX1 polypeptide in inhibiting the production or accumulation of triacylglycerol, preferably in inhibiting DIESL polypeptide mediated triacylglycerol accumulation.

33. The use according to claim 32 wherein the TMX1 polypeptide and/or the a TMX1 polynucleotide that encodes the TMX1 polypeptide is comprised in a cell that further comprises a DIESL polypeptide and/or a DIESL polynucleotide that encodes the DIESL polypeptide.

34. A screening method for identifying mutants of a TMX1 polypeptide, wherein said mutant has altered modulation of DIESL mediated triacylglycerol synthase activity, wherein the method comprises
(a) providing a mutant of theTMX1 polypeptide and providing a DIESL polypeptide;
(b) allowing the mutant TMX1 polypeptide to contact the DIESL polypeptide, preferably under conditions that allow the DIESL polypeptide to produce triacylglycerol; and
(c) detecting activity of the mutant TMX1 polypeptide compared to a control and/or detecting activity of the DIESL polypeptide compared to control to determine the mutant of the TMX1 polypeptide's altered regulation of DIESL mediated triacylglycerol synthase activity.

35. Use of an oligonucleotide to knock down or knock out DIESL and/or TMX1 for modulating triglyceride production in a cell or organism, preferably wherein the use comprises CRISPR/Cas technology, RNAi or antisense technology.

36. A cell that:
is a non-animal cell that expresses a DIESL polypeptide and/or a TMX1 polypeptide;
is a non-human cell that expresses human DIESL polypeptide and/or TMX1 polypeptide;
is a cell that has been modified to not express a DIESL polypeptide and/or a TMX1 polypeptide, and that has been modified to not express a DGAT1 polypeptide;
is a cell that has been modified to not express a DIESL polypeptide and/or a TMX1 polypeptide, and that has been modified to not express a DGAT2 polypeptide;
is a cell that has been modified to not express a DIESL polypeptide and/or a TMX1 polypeptide, and that has been modified to not express a DGAT1 polypeptide and a DGAT2 polypeptide.

37. A non-human transgenic knockout animal, in which the gene encoding for a DIESL polypeptide is knocked out, preferably wherein the polypeptide has at least 75% amino acid sequence identity with the amino acid sequence as defined in SEQ ID NO:1 and exhibits triacylglycerol synthase activity.

38. The non-human transgenic knockout animal according to claim 37, wherein the gene encodes for a DIESL polypeptide that comprises an amino acid sequence having at least 90% sequence identity to SEQ ID NO:1, having at least from 91% to 95% sequence identity to SEQ ID NO:1, or is transmembrane protein 68.

39. The non-human transgenic knockout animal according to any of claims 37-38, wherein the non-human animal is a mouse.

40. The non-human transgenic knockout animal according to claim 39, wherein the knocked out gene that encodes the DIESL polypeptide is the gene with accession number Gene ID 72098.


SEQUENCE LISTING (12 sequences)

SEQ ID NO: 1 - protein, 324 aa, Homo sapiens
MIDKNQTCGVGQDSVPYMICLIHILEEWFGVEQLEDYLNFANYLLWVFTPLILLILPYFTIFLLYLTIIFLHIYKRKNVLKEAYSHNLWDGARKTVATLWDGHAAVWHGYEVHGMEKIPEDGPALIIFYHGAIPIDFYYFMAKIFIHKGRTCRVVADHFVFKIPGFSLLLDVFCALHGPREKCVEILRSGHLLAISPGGVREALISDETYNIVWGHRRGFAQVAIDAKVPIIPMFTQNIREGFRSLGGTRLFRWLYEKFRYPFAPMYGGFPVKLRTYLGDPIPYDPQITAEELAEKTKNAVQALIDKHQRIPGNIMSALLERFH

SEQ ID NO: 2 - protein, 280 aa, Homo sapiens
MAPSGSLAVPLAVLVLLLWGAPWTHGRRSNVRVITDENWRELLEGDWMIEFYAPWCPACQNLQPEWESFAEWGEDLEVNIAKVDVTEQPGLSGRFIITALPTIYHCKDGEFRRYQGPRTKKDFINFISDKEWKSIEPVSSWFGPGSVLMSSMSALFQLSMWIRTCHNYFIEDLGLPVWGSYTVFALATLFSGLLLGLCMIFVADCLCPSKRRRPQPYPYPSKKLLSESAQPLKKVEEEQEADEEDVSEEEAESKEGTNKDFPQNAIRQRSLGPSLATDKS

SEQ ID NO: 3 - DNA, 23 bp, synthetic construct
  misc_feature 1..23: note = artificial
  source 1..23: mol_type = other DNA
ccgacatggtgcttgttgtcctc

SEQ ID NO: 4 - DNA, 21 bp, synthetic construct
  misc_feature 1..21: note = artificial
  source 1..21: mol_type = other DNA
gcaacctccccttctacgagc

SEQ ID NO: 5 - DNA, 20 bp, synthetic construct
  source 1..20: mol_type = other DNA
gctcccttccatttactctg

SEQ ID NO: 6 - DNA, 20 bp, synthetic construct
  source 1..20: mol_type = other DNA
ccggtgagatagctaacaag

SEQ ID NO: 7 - DNA, 20 bp, synthetic construct
  source 1..20: mol_type = other DNA
cttatcatgtctggatccgg

SEQ ID NO: 8 - DNA, 20 bp, synthetic construct
  source 1..20: mol_type = other DNA
ccggtgagatagctaacaag

SEQ ID NO: 9 - DNA, 26 bp, synthetic construct
  source 1..26: mol_type = other DNA
gggcataggaagcaaataccaagttg

SEQ ID NO: 10 - DNA, 24 bp, synthetic construct
  source 1..24: mol_type = other DNA
gtagggccaactgtttctgcatga

SEQ ID NO: 11 - DNA, 25 bp, synthetic construct
  source 1..25: mol_type = other DNA
gtggaattccgctgagagaactctt

SEQ ID NO: 12 - DNA, 24 bp, synthetic construct
  source 1..24: mol_type = other DNA
gtagggccaactgtttctgcatga